(19) United States
(12) Patent Application Publication
(10) Pub. No.: US 2007/0286805 A1
(43) Pub. Date: Dec. 13, 2007

(54) Methods of Validating Target for Modulating Insulin Action, Screening for Modulators of Insulin Action and Therapeutic Uses Thereof

(75) Inventors: David James, Clontarf (AU); Gregory James Cooney, Marrickville (AU); Juan Carlos Molero-Navajas, Coogee (AU)

Correspondence Address: QUINE INTELLECTUAL PROPERTY LAW GROUP, P.C., P O BOX 458, ALAMEDA, CA, 94501, US

(73) Assignee: Garven Institute of Medical Research, New South Wales (AU)

(21) Appl. No.: 10/579,125

(22) PCT Filed: Nov. 15, 2004
(86) PCT No.: PCT/AU04/01572

§ 371 Date: Jul. 31, 2007

(30) Foreign Application Priority Data
Nov. 14, 2003 (AU) 2003906286
Nov. 14, 2003 (AU) 2003906285

(51) Int. Cl. C12Q 1/54; A61K 45/00; A61K 49/00; A61P 3/04; C12Q 1/68; G01N 33/573; A61P 3/10; A61P 9/00; C12Q 1/48

(52) U.S. Cl. 424/9.2; 435/14; 435/15; 435/6; 435/7.4; 514/789

(57) ABSTRACT

The present invention provides several assay formats for determining the effect of compounds on one or more metabolism-associated phenotypes in a cell, tissue or animal. The assay formats are based upon the finding that Cbl (e.g., the protooncogene c-Cbl) is an upstream regulator of AMPK activity and ACC activity in muscle, and that this regulatory pathway modifies whole body energy expenditure and whole body insulin sensitivity.

### FIELD OF THE INVENTION

The present invention relates generally to the field of medicine and more particularly to drug screening processes and to therapeutic products and processes.

### BACKGROUND TO THE INVENTION

1. General

This specification contains nucleotide and amino acid sequence information prepared using patent in Version 3.1, presented herein after the claims. Each nucleotide sequence is identified in the sequence listing by the numeric indicator <210> followed by the sequence identifier (e.g. <210>1, <210>2, <213> etc). The length and type of sequence (DNA, protein (PRT), etc), and source organism for each nucleotide sequence, are indicated by information provided in the numeric indicator fields <211>, <212> and <213>, respectively. Nucleotide sequences referred to in the specification are defined by the term “SEQ ID NO:”, followed by the sequence identifier (eg. SEQ ID NO: 1 refers to the sequence in the sequence listing designated as <400>1).

The designation of nucleotide residues referred to herein are those recommended by the IUPAC-IUB Biochemical Nomenclature Commission, wherein A represents Adenine, C represents Cytosine, G represents Guanine, T represents thymine, Y represents a pyrimidine residue, R represents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

As used herein the term “derived from” shall be taken to indicate that a specified integer may be obtained from a particular source albeit not necessarily directly from that source.

The embodiments of the invention described herein with respect to any single embodiment shall be taken to apply mutatis mutandis to any other embodiment of the invention described herein.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific embodiments described herein, which are intended for the purposes of exemplification only. Functionally equivalent products, compositions and methods are clearly within the scope of the invention, as described herein.

The present invention is performed without undue experimentation using, unless otherwise indicated, conventional techniques of molecular biology, microbiology, virology, recombinant DNA technology, peptide synthesis in solution, solid phase peptide synthesis, and immunology. Such procedures are described, for example, in the following texts that are incorporated by reference:
1. Sam brook, Fritsch & Maniatis, Molecular Cloning: A Laboratory Manual, Cold Spring Harbor Laboratories, New York, Second Edition (1989), whole of Vols I, II, and III;
2. DNA Cloning: A Practical Approach, Vols. I and II (D. N. Glover, ed., 1985), IRL Press, Oxford, whole of text;
3. Oligonucleotide Synthesis: A Practical Approach (M. J. Gait, ed., 1984) IRL Press, Oxford, whole of text, and particularly the papers therein by Gait, pp 1-22; Atkinson et al., pp 35-81; Sproat et al., pp 83-115; and Wu et al., pp 135-151;
4. Nucleic Acid Hybridization: A Practical Approach (B. D. Hames & S. J. Higgins, eds., 1985) IRL Press, Oxford, whole of text;
5. Animal Cell Culture: Practical Approach, Third Edition (John R. W. Masters, ed., 2000), ISBN 0199637970, whole of text;
6. Immobilized Cells and Enzymes: A Practical Approach (1986) IRL Press, Oxford, whole of text;
7. Perbal, B., A Practical Guide to Molecular Cloning (1984);
8. Methods In Enzymology (S. Colowick and N. Kaplan, eds., Academic Press, Inc.), whole of series;
9. J. F. Ramalho Ortigão, “The Chemistry of Peptide Synthesis” In: Knowledge database of Access to Virtual Laboratory website (Interactiva, Germany);
10. Sakakibara, D., Teichman, J., Lien, E. Land Fenichel, R. L. (1976). Biochem. Biophys. Res. Commun. 73 336-342
11. Merrifield, R. B. (1963). J. Am. Chem. Soc. 85, 2149-2154.
12. Barany, G. and Merrifield, R. B. (1979) in The Peptides (Gross, E. and Meienhofer, J. eds.), vol. 2, pp. 1-284, Academic Press, New York.
13. Wünsch, E., ed. (1974) Synthese von Peptiden in Houben-Weyls Metoden der Organischen Chemie (Müler, E., ed.), vol. 15, 4th edn., Parts 1 and 2, Thieme, Stuttgart.
14. Bodanszky, M. (1984) Principles of Peptide Synthesis, Springer-Verlag, Heidelberg.
15. Bodanszky, M. & Bodanszky, A. (1984) The Practice of Peptide Synthesis, Springer-Verlag, Heidelberg.
16. Bodanszky, M. (1985) Int. J. Peptide Protein Res. 25, 449-474.
17. Handbook of Experimental Immunology, Vols. I-IV (D. M. Weir and C. C. Blackwell, eds., 1986, Blackwell Scientific Publications).

2. Description of the Related Art

The c-Cbl protein is a multi-adaptor protein that is involved in ligand-induced down regulation of receptor tyrosine kinases. The interaction between c-Cbl and its many binding partners involves particular c-Cbl protein domains. All Cbl proteins have a high degree of sequence homology between their tyrosine kinase-binding, linker and RING finger domains, and most have extensive proline-rich regions in their carboxy-terminal halves. The tyrosine kinase-binding domain is composed of three interacting domains comprising a four-helix bundle, a Ca²⁺-binding EF hand, and an atypical or variant Src homology region 2 (SH2) domain that is connected to the RING finger by a short linker domain. A ubiquitin-associated (UBA)/LZ domain at the carboxyl terminus of c-Cbl, Cbl-b and D-Cbl has homology to known ubiquitin-associated domains and leucine zippers and is believed to be involved in Cbl-mediated ubiquitination of active receptors, which is essential for receptor degradation and turnover, thereby leading to cessation of downstream signalling from the receptor (Soubeyran et al., Nature 416, 183-187, 2002).

Diabetes, and conditions related thereto such as atherosclerosis, heart disease, obesity, are major health concerns throughout the world, and contribute to morbidity and mortality. Non-insulin dependent diabetes mellitus (NIDDM or type II diabetes), is the major form of diabetes in developed countries, however efficient means of therapeutic intervention are lacking. While a large number of environmental and genetic factors contribute to the risk of NIDDM in the United States, prolonged obesity is by far the largest risk factor.

A variety of factors contribute to insulin resistance in laboratory animals, as well as in humans, including a sedentary lifestyle, a diet high in either fat or carbohydrate. A common feature of these risk factors is that the energy balance shifts in favour of energy storage usually in the form of lipid. The intracellular accumulation of lipid is toxic to many cells such as pancreatic p-islet cells and this is considered to be an important factor in the progression of NIDDM.

To address these problems, the pharmaceutical industry has focused its efforts on identifying compounds that target a variety of metabolic endpoints, including insulin resistance, food intake and nutrient absorption. However, relatively few drugs have made it through clinical trials and most of these have unpleasant side effects. Insulin-sensitizing compounds that have been identified to target insulin resistance associated with both obesity and NIDDM often lead to increased body weight and so ultimately these may exacerbate some of the problems associated with these disorders. The insulin-sensitising compounds appear to increase adipocyte differentiation.

Before the development of diabetes, many obese patients develop a peripheral resistance to the actions of insulin. The molecular basis of insulin-resistance in obesity has been the subject of intensive study, but nonetheless remains elusive. Insights into components and mechanisms of the link between obesity and insulin resistance have been gained from mouse models of obesity which display obesity-induced insulin resistance. The molecular basis of the various mouse obesity models covers a range of mechanisms; nonetheless these all develop diabetes, either before or after the onset of obesity.

Obesity in humans and rodents is also commonly associated with insulin resistance in fat and muscle cells (LeRoith et al., Diabetes Mellitus: a Fundamental and Clinical Text. (Lippincott-Raven, Philadelphia, 1996); DeFronzo et al., Diabetes Care 15:318-68 (1992); Rifkin et al., Diabetes Mellitus, (Elsevier, N.Y., 1990)).

Much work has focussed on the insulin-sensitive glucose transporter GLUT4 (Watson and Pessin, Exp. Cell Res. 271: 75-83, 2001; Bogan et al. published U.S. Patent Application No. 20020052012). Insulin binds to the insulin receptor (IR) in the plasma membrane, where it activates tyrosine kinase in a cascade of events involving phosphatidylinositol 3-kinase (PI 3-K)-mediated recruitment of GLUT4 to the cell surface Min et al., Mol. Cell. 3: 751-760, 1999; Olson et al., Mol. Cell. Biol. 17: 2425-2435, 1997; Hausdorff et al., J. Biol. Chem. 270: 12965-12968, 1995; Elmendorf et al., J. Biol. Chem. 273: 13289-13296, 1998; Holman et al., J. Biol. Chem. 265: 18172-18179, 1990; Piper et al., Am. J. Physiol. 260: C570-C580). The activation of PI 3-K However, PI-K3-mediated trafficking of GLUT4 is not sufficient to explain the extent of insulin resistance (Pessin et al., J. Clin. Invest. 106: 165-169, 2000). For example, other growth factors and adhesion molecules that can activate PI 3-K and its downstream kinases (ie. AKT, protein kinase Cζ/λ (PKCζ/λ)) have no effect on glucose transport or GLUT4 translocation. Additionally, mutant mice lacking GLUT4 develop only mild hyperinsulinemia (Katz et al., Nature 377:151-155, 1995).

A second PI 3K-independent signalling cascade, initiated by recruitment of c-Cbl (Langdon et al., J. Virol. 63: 5420-5424, 1989; Langdon et al., Proc. Natl Acad. Sci. USA 86: 1168-1172, 1989) to the insulin receptor, has been postulated to be involved in insulin-stimulated glucose transport and uptake in fat and muscle cells. In this pathway, the c-Cbl protein is also recruited to the insulin receptor by interaction with the adaptor protein CAP, through one of three SH3 domains in the carboxyl-terminus of CAP. c-Cbl is then phosphorylated by the receptor, and the CAP-Cbl complex dissociates from the insulin receptor and moves to a caveolin-enriches triton insoluble membrane fraction. Based upon two-hybrid assay data measuring in vitro protein-protein associations Baumann et al., Nature 407: 202-207, 2000, showed that c-Cbl forms a ternary complex with two other proteins, CAP and flotillin. The interaction with flotillin directs the CAP-Cbl complex to the lipid raft sub-domain of the plasma membrane. Baumann et al. Nature 407: 202-207, 2000, also showed that both insulin-stimulated glucose transport and GLUT4 translocation to the cell surface are attenuated by about 50% in 3T3-L1 adipocytes by expression of a truncated CAP protein lacking SH3 domains (i.e. CAPΔSH3). If this pathway were to operate in fat cells and/or muscle cells in vivo, it would be expected that insulin-induced glucose uptake and its subsequent incorporation into both glycogen and lipid would be impaired in situations which disrupt formation or activity of the c-Cbl-CAP-flotillin complex.

An interaction between c-Cbl and APS, an adaptor protein having a PH domain and SH2 domain (Ahmed et al., Biochem. J. 341, 665-668, 1999) is required for c-Cbl to bind to the insulin receptor in the caveolae-small invaginations in the plasma membrane that are a subset of the lipid raft domains. In this pathway, APS interacts with the phosphorylated insulin receptor via its SH2 domain, and subsequently undergoes tyrosine phosphorylation at a specific residue in the C-terminus of the protein. Upon phosphorylation, APS recruits c-Cbl to the receptor through an atypical SH2 domain of c-Cbl (Saltiel and Pessin, TRENDS Cell Biol. 12, 65-71, 2002).

Based upon co-localization studies, Chiang et al., Nature 410: 944-948, 2001 also showed that the time-course for the insulin-stimulated migration of c-Cbl parallels movement of the SH2-containing adaptor protein CrkII, and the guanyl nucleotide exchange factor C3G, into the caveolin-enriches triton insoluble membrane fraction. Chiang et al. also demonstrated that C3G exchanges GTP for GDP on TC10, a Rho-family GTP-binding protein that regulates GLUT4 transport. In this pathway, phosphorylated c-Cbl recruits CrkII to the lipid rafts, along with C3G to facilitate activation of TC10 by C3G (Chiang et al., Nature 410: 944-948, 2001).

No direct role for c-Cbl in modulating glucose uptake or GLUT4 translocation has been demonstrated in muscle and fat cells in vivo. This is because the binding studies by Baumann et al. and Chiang et al. supra were carried out in isolated 3T3L1 adipocytes and do not suggest that equivalent effects occur in vivo, let alone in both muscle and fat cells of animals. It is also unclear whether the effects reported by Baumann et al. for the over expression of CAPΔSH3 protein in isolated adipocytes were a direct consequence of CAP failing to bind c-Cbl or to a secondary effect of expressing the mutant CAPΔSH3 protein. For example, the expressed CAPΔSH3 protein may have modified the ability of endogenous CAP to bind other proteins in the lipid rafts or elsewhere in the cell. Over expression of the CAPΔSH3 protein also decreases insulin-stimulated recruitment of C3G into lipid rafts, and reduces the basal level of activated TC10 in 3T3-L1 adipocytes (Chiang et al., Nature 410: 944-948, 2001). Accordingly, no clear direct role has emerged for c-Cbl in modulating glucose uptake and incorporation into lipid in both fat cells and muscle cells of animals. Thus, it is not possible at present to conclude that impaired c-Cbl-CAP-flotillin complex formation and activity are sufficient to produce insulin resistance in humans and other animals.

A number of mouse models have been developed having genetic obesity-diabetes syndromes (Herberg, et al., Metabolism 26: 59-99, 1977). The mice typically have hyperglycemia, hyperinsulinemia, and obesity, albeit to different degrees, with different times of onset, and for different reasons. In the yellow obese mouse (A^{y/a}), a dominant mutation causes the ectopic, ubiquitous expression of the agouti protein, resulting in a condition similar to adult-onset obesity and non-insulin-dependent diabetes mellitus (Michaud et al., Proc Natl Acad Sci USA 91: 2562-2566, 1994). Obese (ob/ob) (Zhang et al., Nature 372: 425-432, 1994); diabetes (db/db) (Tartaglia et al., Cell 83: 1263-1271, 1995), adipose (cpe/cpe) (Naggert et al., Nat. Genet. 10: 135-142, 1995) and tubby (tub/tub) (Kleyn et al., Cell 85: 281-290, 1996; Noben-Trauth et al., Nature 380: 534-548, 1996) are mutations in the genes encoding leptin, the leptin receptor, carboxypeptidase E, and a member of a new family of genes encoding tubby-like proteins, respectively. Obese mice exhibit hyperglycemia, glucose intolerance, and elevated plasma insulin, which develops after the onset of obesity. In db/db mice, elevation of plasma insulin occurs at 2 weeks of age, preceding the onset of obesity at 3-4 weeks and elevation of blood glucose levels at 4-8 weeks. Adipose mice have hyperinsulinemia throughout life in association with hypertrophy and hyperplasia of the islets of Langerhans, with transient hyperglycemia. Tubby mice have normal blood glucose, however plasma insulin is elevated prior to obvious signs of obesity, and the islets of Langerhans are enlarged.

Mouse models having impaired glucose uptake are highly desirable. By virtue of examining their phenotype, such models would have utility in determining appropriate targets for the therapy of a wide range of disorders associated with aberrant glucose metabolism and for determining the efficacy or specificity of therapeutics.

### SUMMARY OF THE INVENTION

The present invention is predicated on the phenotype of a Cbl-deficient animal model, which the inventors have found possesses modified feeding behavior, fat content, muscle mass, insulin action and downstream effects thereof. The present invention provides methods for identifying therapeutic targets for the treatment of conditions associated with aberrant insulin action, such as, for example, insulin resistance, glucose intolerance, diabetes, obesity, atherosclerosis and heart disease generally, and methods of identifying compounds that modulate insulin action by virtue of one or more effects on the activity and/or expression of said therapeutic targets. The invention further provides compounds identified using the novel screening processes of the invention and to therapeutic methods for the treatment of conditions associated with aberrant insulin action using the compounds.

As used herein, the term “Cbl-deficient” is meant that expression of functional Cbl protein is reduced such that the level or activity of one or more downstream effectors of Cbl, especially c-Cbl, is modified compared to otherwise isogenic animals, tissues or cells expressing wild-type levels of functional Cbl protein. The downstream effectors modulated by Cbl include, but are not limited to, any of the phenotypes of a Cbl^{-/-} mouse in which expression of one or more alleles of a c-Cbl locus has been inactivated by targeted disruption of an endogenous c-Cbl gene, or cells in which expression of c-Cbl has been knocked-down using an inhibitor of c-Cbl expression e.g., RNAi targeting expression of c-Cbl. Phenotypes associated with Cbl-deficiency include, but are not limited to reduced fat content, increased lean muscle mass, increased muscle thermogenesis, increased feeding behavior, modified insulin action including increased insulin receptor (IR) levels and increased glucose tolerance, increased fatty acid oxidation, and increased activities of certain mitochondrial enzymes. Such phenotypes can readily be determined using known methods, such as, for example, by determining 2-deoxyglucose uptake into isolated liver, fat or muscle cells and/or glycogen synthesis and/or lipogenesis in the presence and absence of insulin as described by Lazar et al., J. Biol. Chem. 270: 20801-20807, 1995.

For example, a genetic modification capable of producing a Cbl deficient phenotype is selected from the group consisting of a deletion, an insertion, a substitution and an inversion of nucleotides in an allele of a Cbl locus. A deletion of a nucleotide sequence within two alleles of the Cbl locus, wherein the deletion results in an absence of expression of a functional or full-length Cbl protein by the animal, can also be performed to produce Cbl-deficiency. Deletion of a nucleotide sequence encoding a protein-encoding portion of a Cbl gene, or alternatively, the introduction of an in-frame stop codon at a location in the protein-encoding portion of a Cbl gene are preferred methods for producing Cbl-deficiency. However, targeted disruption of one or two alleles at the Cbl locus thereby producing a truncated Cbl protein comprising the amino acid sequence set forth in SEQ ID NO: 1 is particularly preferred for producing Cbl-deficiency.

Disrupted expression of both endogenous Cbl alleles at the Cbl locus is preferred, however heterozygosity also produces Cbl-deficiency.

Cbl deficiency should be evident at least in liver, fat or muscle cells, more preferably in a tissue selected from the group consisting of adipose, skeletal muscle and cardiac muscle. Effects of Cbl deficiency in other cells or tissues, such as, for example, immune cells or brain, are not to be excluded from the scope of the term “Cbl-deficient”.

As used herein, the term “Cbl” shall be taken to mean any peptide, polypeptide, or protein having at least about 80% amino acid sequence identity to the amino acid sequence of a human or mouse c-Cbl polypeptide set forth in SEQ ID NO: 2 or 3. The term “Cbl” shall also be taken to include a peptide, polypeptide or protein having the known biological activity of Cbl, or the known binding specificity of Cbl including c-Cbl. For the purposes of nomenclature, the amino acid sequences of the murine and human Cbl polypeptides are exemplified herein, as SEQ ID Nos: 2 and 3, respectively. Preferably, the percentage identity to SEQ ID NO: 2 or 3 is at least about 85%, more preferably at least about 90%, even more preferably at least about 95% and still more preferably at least about 99%.

A “Cbl” protein generally comprises about 906 amino acid residues in length. The full-length protein generally comprises a tyrosine kinase-binding domain that binds the protein to phosphotyrosine residues, thereby coupling Cbl to growth factor receptor signalling. For example, the c-Cbl is phosphorylated on 3-4 tyrosine residues in response to growth factors, the phosphorylated residues representing binding domains for SH2 domain containing proteins such as PI 3′ kinase and Crk II. The tyrosine kinase-binding domain generally will comprise a four-helix bundle, an EF hand domain and an SH2 domain. Downstream of the tyrosine kinase-binding domain is generally a C3HC4 RING finger domain having high sequence similarity to that found in ubiquitin ligase proteins. At the C terminal portion of the protein there is generally a proline rich region (PRR) that can bind SH3 domain-containing proteins (e.g. CAP). At the extreme C terminus of Cbl there is generally located a leucine zipper domain (b-Zip), and ubiquitin association domain that regulates homodimerization of Cbl.

The inventors found that a Cbl-deficient mouse exhibited about 50% reduction in major adipose tissue beds and whole body fat content, notwithstanding about 30% increased appetite as determined by ingestion of feed, correlated with reduced fasting leptin i.e., hypoleptinemia, and enhanced neuropeptide Y in the arcuate nucleus, compared to wild-type animals. Thus, animals in which Cbl expression is reduced have enhanced lean muscle mass relative to fat content.

The inventors also found that the “lean” Cbl-deficient phenotype is explained, at least in part, by enhanced or increased insulin action, i.e., improved glucose tolerance combined with reduced insulin level compared to wild-type animals as determined by standard glucose tolerance test. More particularly, this increased blood glucose clearance at lower insulin levels indicates that peripheral insulin action is enhanced when Cbl expression is reduced. Little or no defect on basal or insulin-induced glucose uptake was observed. Nor was there any significant difference in the basal or insulin-stimulated incorporation of glucose into lipid in adipocytes. In contrast, clearance of glucose into glycogen was enhanced in muscle, e.g., soleus and extensor digitorum longus (EDL) muscle from animals having reduced Cbl expression, compared to wild-type animals. The enhanced insulin sensitivity of animals in which Cbl expression is reduced is also partly explained by a marked enhancement or increase in the number of insulin receptors (IR) in muscle and adipose tissue i.e., the level of insulin receptor (IR) is enhanced or increased relative to wild type animals.

The “lean” phenotype and enhanced insulin sensitivity of animals in which Cbl expression is down-regulated are explained, at least in part, by an enhancement of whole body energy expenditure or metabolic rate, i.e., the animals have increased nocturnal ambulatory activity, increased body temperature of about 1° C., enhanced muscle thermogenesis and enhanced whole-body oxygen consumption compared to otherwise isogenic wild-type animals. The respiratory quotient (RQ) is also significantly reduced in Cbl-deficient animals, suggesting that the enhanced energy expenditure is supported by enhanced fatty acid oxidation. Consistent with the reduced RQ, animals in which expression of Cbl is reduced have significantly lower levels of triglycerides in liver, and free fatty acids (FFA) in plasma compared with wild-type control animals.

To explain the enhanced energy expenditure and metabolic rate in animals having reduced Cbl expression, the inventors investigated mitochondrial function in the skeletal muscle of the animals. No modifications were found in sarcolemma area, crista density, or levels of the mitochondrial protein PGC1α (which is known to be involved in mitochondrial biogenesis and FFA metabolism; Wu et al., Cell 98, 115-124, 1999; Lehman et al., J. Clin. Invest, 106, 847-856, 2000). However, the inventors found that the Cbl-deficient animals exhibited moderately enhanced levels of acetyl-coA carboxylase (ACC) and uncoupling protein-3 (UCP3). Notwithstanding the moderately enhanced level in total ACC, there was a marked (about 4-fold) increase in the level of phosphorylated ACC in the muscle of Cbl-deficient animals, indicating an overall reduction in ACC activity in this population of animals. To explore the mechanism of increased ACC phosphorylation, the inventors investigated the expression and phosphorylation state of AMP-activated protein kinase (AMPK), and demonstrated about 70% higher levels of phosphorylated AMPK (pAMPK) with no significant change in the total level of AMPK in muscle.

The inventors have now also shown that Cbl (e.g., the protooncogene c-Cbl) is an upstream regulator of AMPK activity and ACC activity in muscle, and that this regulatory pathway modifies whole body energy expenditure and whole body insulin sensitivity. Without being bound by any theory or mode of action, the newly-elucidated role of c-Cbl in controlling turnover of protein tyrosine kinase and/or protein tyrosine kinase activity also suggests that a tyrosine kinase is involved in regulating body weight.

The elucidation of the above regulatory pathway by the inventors provides the basis for new processes for the identification of compounds that modulate whole body energy expenditure and whole body insulin sensitivity in animals. Compounds identified in the screens described herein are useful for modifying glucose uptake and/or in the treatment of hypoglycemia and/or hyperglycemia, and/or disorders associated with aberrant whole body energy expenditure and/or aberrant whole body insulin sensitivity such as, for example, obesity, diabetes, glucose intolerance, insulin resistance, hypolipidemia (e.g. as observed in subjects suffering from abetalipoproteinemia, malnutrition or hematologic malignancies, such as acute myelocytic leukemia or chronic myelocytic leukemia), atherosclerosis and heart disease generally. By virtue of the effect of modifying Cbl expression on the ratio of lean muscle mass to body fat, such compounds are also useful for cosmetic applications e.g., body building, dieting, etc. These processes include in vitro, in situ and in vivo assays for determining expression of ACC protein and/or phospho-ACC protein and/or mRNA encoding ACC, AMPK protein and/or phospho-AMPK protein and/or mRNA encoding AMPK, FFA level, FFA oxidation, and RQ. For example, candidate compounds are screened to determine their effect on a suitable animal model, e.g., a Cbl^{-/-} mouse model or a derivative thereof expressing human c-Cbl protein, or in intact cells e.g., myoblasts, or by direct screening of cell or tissue extracts, for modifications to any of these parameters in the presence and absence of the compounds. Data presented in FIGS. 12a and 12b also show comparable results for Cbl knockdown in isolated cells and Cbl-deficient animals, thereby validating the utility of isolated cells, e.g., myoblasts, for high-throughput compound screening to identify therapeutic agents. For example, the enhanced levels of insulin receptor and phosphorylated ACC and AMPK seen in the Cbl-deficient animal model are reproduced by transfecting isolated cells with shRNA targeting Cbl expression (FIGS. 12a and 12b) in which Cbl expression is knocked-down by about 95%.

Accordingly, the present invention provides a method of identifying a compound that reduces whole body insulin sensitivity of an animal, such as, for example, in the treatment of hypolipidemia (e.g. as observed in subjects suffering from abetalipoproteinemia, malnutrition or hematologic malignancies, such as acute myelocytic leukemia or chronic myelocytic leukemia), said method comprising: (a) administering a compound to a non-human animal, isolated tissue or isolated cell having reduced expression of functional Cbl compared to an otherwise isogenic wild-type animal, tissue or cell; and (b) determining the activity of ACC and/or amount of phosphorylated ACC enzyme in the animal, tissue or cell wherein a reduced amount of phosphorylated ACC enzyme and/or enhanced activity of an ACC enzyme compared to the amount of phosphorylated ACC enzyme or activity of an ACC enzyme of a Cbl-deficient animal, tissue or cell to which the compound has not been administered indicates that the compound reduces whole body insulin sensitivity in an animal.

In an alternative embodiment, the invention provides a method of identifying a compound that enhances whole body insulin sensitivity of an animal comprising: (a) administering a compound to a non-human animal, isolated tissue or isolated cell expressing a functional Cbl protein and determining the activity of ACC and/or amount of phosphorylated ACC enzyme; (b) determining the activity of ACC and/or amount of phosphorylated ACC enzyme in a non-human animal, isolated tissue or isolated cell having reduced expression of functional Cbl compared to an otherwise isogenic wild-type animal, tissue or cell; and (c) comparing the activity of ACC and/or amount of phosphorylated ACC enzyme at (a) and (b) wherein a comparable activity and/or amount of phosphorylated ACC enzyme between (a) and (b) indicates that the compound enhances whole body insulin sensitivity of an animal.

As used herein the term “ACC” or “acetyl CoA carboxylase” shall be taken to include all forms of acetyl CoA carboxylase, for example, an ACC1 or an ACC2 polypeptide. Preferably, an ACC is an ACC2 protein. Even more preferably, the term “ACC” shall be taken to mean any peptide, polypeptide or protein having at least about 80% amino acid sequence identity to the amino acid sequence of human ACC2 set forth in SEQ ID NO: 4 or a fragment thereof. The term “ACC” shall also be taken to include a peptide, polypeptide or protein having a known biological activity of an ACC.

An ACC protein, and, in particular, an ACC2 protein comprises a signal peptide targeting the ACC protein to a mitochondrial membrane. Without been bound by theory it is thought that ACC2 produces malonyl-CoA that is a regulator of the carnitine/palmitoyl-CoA shuttle system associated with the mitochondrial membrane.

By “phosphorylated ACC” is meant that an ACC polypeptide as described herein has had a phosphate group covalently attached to one or more amino acids within the polypeptide. Preferably, the phosphate group is attached to one or more serine residues within the ACC polypeptide, e.g., Ser79.

ACC activity is readily determined by CO₂ fixation, i.e., by measuring the incorporation of labeled carbon into malonyl CoA in the presence and absence of the compound. ACC activity can also be determined using an immunoassay wherein the level of phosphorylated ACC is determined by contacting cells or a cell extract with an antibody that binds to the phosphorylated form of the enzyme under conditions sufficient for an antigen-antibody complex to form and detecting the antibody bound. Preferably, the assay further comprises separately contacting the cells or cell extract with an antibody that binds to non-phosphorylated ACC under conditions sufficient for an antigen-antibody complex to form and detecting the antibody bound, thereby facilitating determination of the percentage of phosphorylated ACC relative to total ACC in the sample. Preferably, the antibody bound to the ACC protein is detected by contacting the antibody with a secondary antibody that is capable of producing a detectable signal. A change in activity is then determined in the presence and absence of the compound being tested.

The present invention also provides a method of identifying a compound that reduces whole body insulin sensitivity of an animal, such as, for example, in the treatment of hypolipidemia (e.g. as observed in subjects suffering from abetalipoproteinemia, malnutrition or hematologic malignancies, such as acute myelocytic leukemia or chronic myelocytic leukemia), said method comprising: (a) administering a compound to a non-human animal, isolated tissue or isolated cell having reduced expression of functional Cbl compared to an otherwise isogenic wild-type animal, tissue or cell; and (b) determining the activity of AMPK and/or amount of phosphorylated AMPK enzyme in the animal, tissue or cell wherein a reduced amount of phosphorylated AMPK enzyme and/or reduced activity of AMPK enzyme compared to the amount of phosphorylated AMPK enzyme or activity of AMPK enzyme of a Cbl-deficient animal, tissue or cell to which the compound has not been administered indicates that the compound reduces whole body insulin sensitivity in an animal.

In an alternative embodiment, the invention provides a method of identifying a compound that enhances whole body insulin sensitivity of an animal comprising: (a) administering a compound to a non-human animal, isolated tissue or isolated cell expressing a functional Cbl protein and determining the activity of AMPK and/or amount of phosphorylated AMPK enzyme; (b) determining the activity of AMPK and/or amount of phosphorylated AMPK enzyme in a non-human animal, isolated tissue or isolated cell having reduced expression of functional Cbl compared to an otherwise isogenic wild-type animal, tissue or cell; and (c) comparing the activity of AMPK and/or amount of phosphorylated AMPK enzyme at (a) and (b) wherein a comparable activity of AMPK and/or comparable amount of phosphorylated AMPK enzyme between (a) and (b) indicates that the compound enhances whole body insulin sensitivity of an animal.

As used herein the term “AMPK” or “AMP-activated protein kinase” or similar term shall be taken to include all forms of AMP-activated protein kinase having the metabolic or enzymatic activity of mammalian AMP-activated protein kinase, including any non-mammalian homolog. The skilled artisan is aware that mammalian AMPK is a trimeric enzyme composed of a catalytic α subunit and the non-catalytic β and γ subunits. There are two genes encoding isoforms of both α and β subunits (α1, α2, β1 and β2) and three genes encoding isoforms of the γ subunit (γ1-γ3). The α2 isoform is the subunit of AMPK found predominantly within skeletal and cardiac muscle, whereas, approximately equal distribution of both the α1 and α2 isoforms are present in hepatic AMPK. Within pancreatic islet β-cells the α1 isoform predominates. For the present purpose, it is preferred to determine activity of AMPK as regulated by phosphorylation of the enzyme and, in this respect, there is no detectable activity of AMPK towards any substrates in the absence of phosphorylation. Phosphorylation of AMPK occurs in the α1 and α2 subunits at threonine 172 (Thr-172), in the activation loop. Accordingly, for the present purpose, the term “AMPK” shall be taken to mean any peptide, polypeptide or protein comprising an α subunit that comprises an amino acid sequence that is at least about 80% identical to the sequence of the human AMPK α2 subunit set forth in SEQ ID NO: 5 or a fragment thereof.

AMPK activity is readily determined by following the incorporation of labelled phosphate e.g., ³²P, into the synthetic peptide SAMS (Kudo et al., Biochim Biophys Acta 1391, 67-75, 1996) in the presence and absence of the compound. AMPK activity can also be determined using an immunoassay wherein the level of phosphorylated AMPK is determined by contacting cells or a cell extract with an antibody that binds to the phosphorylated form of the enzyme under conditions sufficient for an antigen-antibody complex to form and detecting the antibody bound. Preferably, the assay further comprises separately contacting the cells or cell extract with an antibody that binds to non-phosphorylated AMPK under conditions sufficient for an antigen-antibody complex to form and detecting the antibody bound, thereby facilitating determination of the percentage of phosphorylated AMPK relative to total AMPK in the sample. Preferably, the antibody bound to the AMPK protein is detected by contacting the antibody with a secondary antibody that is capable of producing a detectable signal. A change in activity is then determined in the presence and absence of the compound being tested.

The present invention also provides a method of identifying a compound that reduces whole body insulin sensitivity of an animal, such as, for example, in the treatment of hypolipidemia (e.g. as observed in subjects suffering from abetalipoproteinemia, malnutrition or hematologic malignancies, such as acute myelocytic leukemia or chronic myelocytic leukemia), said method comprising: (a) administering a compound to a non-human animal, isolated tissue or isolated cell having reduced expression of functional Cbl compared to an otherwise isogenic wild-type animal, tissue or cell; and (b) determining free fatty acid level and/or fatty acid oxidation in the animal, tissue or cell, wherein enhanced free fatty acids and/or reduced fatty acid oxidation compared to free fatty acid level and/or fatty acid oxidation of a Cbl-deficient animal, tissue or cell to which the compound has not been administered indicates that the compound reduced whole body insulin sensitivity of an animal.

In an alternative embodiment, the invention provides a method of identifying a compound that enhances whole body insulin sensitivity of an animal, such as, for example, in the treatment of obesity, atherosclerosis or heart disease, or neurodegenerative disorders or for cosmetic purposes such as bodybuilding or weight loss, said method comprising: (a) administering a compound to a non-human animal, isolated tissue or isolated cell expressing a functional Cbl protein and determining free fatty acid level and/or fatty acid oxidation in the animal, tissue or cell; (b) determining free fatty acid level and/or fatty acid oxidation in a non-human animal, isolated tissue or isolated cell having reduced expression of functional Cbl compared to an otherwise isogenic wild-type animal, tissue or cell; and (c) comparing free fatty acid level and/or fatty acid oxidation at (a) and (b) wherein a comparable free fatty acid level and/or fatty acid oxidation between (a) and (b) indicates that the compound enhances whole body insulin sensitivity of an animal.

Standard assays can be used to readily determine levels of free fatty acids and fatty acid oxidation in cells and tissues e.g., using publicly available calorimetric kits.

The assays described herein are readily performed in cells, tissues or organisms other than those specifically described which have modified activity of one or more enzymes in the regulatory pathway of insulin action identified by the present inventors (e.g., IR, AMPK, ACC, FFA, FA oxidation, etc), preferably modified activity in one or more enzymes acting upstream of a readout integer. For example, assays in which ACC phosphorylation or activity or fatty acid oxidation or free fatty acid level is determined as a readout can be conducted in cells, tissues or organisms in which AMPK activity is up-regulated, with or without concomitant down-regulated Cbl activity. Similarly, for determining fatty oxidation or free fatty acid levels, cells, tissues or organisms in which ACC activity is down-regulated and/or AMPK activity is up-regulated, with or without concomitant down-regulated Cbl activity, can be used. Cells in which IR is up-regulated are also contemplated for use herein. However, since it is clearly desirable to mimic one or more Cbl^{-/-} phenotypes described herein, it is clearly preferred to conduct such assays in cells having the opposing phenotype(s) and then assay for compounds that mimic one or more Cbl^{-/-} phenotypes associated with insulin action. Suitable cells, tissues or organisms are readily produced e.g., using RNAi knock-down of endogenous activity of IR and/or AMPK and/or by ectopic over-expression of the open reading frame of a gene encoding ACC. The present invention clearly encompasses such cells, tissues and organisms and uses thereof in the identification of compounds that modulate insulin action.

The present invention further extends to any and all cells, tissues or organisms that mimic c-Cbl knock-out e.g., using RNAi knock-down of endogenous activity of ACC, or conversely, by ectopic over-expression of the open reading frame of a gene encoding IR and/or a gene encoding AMPK, and uses thereof in the identification of compounds that modulate insulin action.

It will be apparent to the skilled artisan from the preceding description that the present inventors have validated c-Cbl is an important upstream regulator of both ACC and AMPK activity in regulating whole body energy expenditure and that muscle mitochondrial function is a key therapeutic target for the treatment of type 2 diabetes, obesity, atherosclerosis or heart disease.

Accordingly, the present invention also provides a method of identifying a therapeutic target for the treatment of aberrant insulin action or a condition associated therewith, such as, for example, insulin resistance, glucose intolerance, type 2 diabetes, obesity, atherosclerosis or heart disease, said method comprising administering to an animal, tissue or cell a compound capable of reducing the expression and/or activity of a Cbl protein and determining the activity and/or expression of one or more genes and/or proteins in the animal, tissue or cell wherein modified expression and/or activity of a gene or protein indicates that the gene or protein is a therapeutic target for the treatment of aberrant insulin action or a condition associated therewith.

Cbl expression at the protein or mRNA level protein can also be determined in surrogate assays for determining mitochondrial activity, especially activity of ACC and/or AMPK and/or UCP3 and/or FFA level, FA oxidation, RQ, or IR levels. Tyrosine phosphorylation activity or ubiquitin ligase activity of Cbl protein can be determined in lieu of Cbl expression. Alternatively, because C-terminally-tyrosine-phosphorylated Cbl binds to the Epidermal Growth Factor Receptor (EGFR) and promotes monoubiquitination of activated EGFR, and also promotes monoubiquitination of CIN85 in complexes with endophilin and activated EGFRs, c-Cbl activity can be measured in vitro using recombinant Epidermal Growth Factor Receptor (EGFR) or a cytosolic portion thereof, or recombinant CIN85, as a substrate for c-Cbl E3 ubiquitin ligase activity, in which case monoubiquitination of such peptide substrates is determined. Assays in which monoubiquination of EGFR and/or CIN85 is determined are particularly preferred for high throughput applications.

The above-mentioned assays are generally performed on nucleic acid-containing or protein-containing extracts derived from animals, tissues or cells having pre-determined levels of Cbl expression in the absence of the compound(s) being tested. The following assays are useful for determining insulin sensitivity and/or energy expenditure of an animal in accordance with the teaching provided herein of the role of Cbl in modulating insulin action and whole body energy expenditure.

For example, the present invention provides a method of identifying a compound that reduces mitochondrial ACC enzyme activity comprising determining the level of Cbl expression and/or activity in the presence and absence of the compound wherein enhanced expression and/or activity of Cbl in the presence of the compound indicates that the compound reduces mitochondrial ACC enzyme activity. Preferably, ACC enzyme activity is reduced by increasing the amount of phosphorylated ACC e.g., by increasing AMPK activity.

The present invention also provides a method of identifying a compound that enhances mitochondrial ACC enzyme activity comprising determining the level of Cbl expression and/or activity in the presence and absence of the compound wherein reduced expression and/or activity of Cbl in the presence of the compound indicates that the compound enhances mitochondrial ACC enzyme activity. Preferably, ACC enzyme activity is enhanced by decreasing the amount of phosphorylated ACC or increasing dephosphorylation of ACC.

The present invention also provides a method of identifying a compound that reduces mitochondrial AMPK enzyme activity comprising determining the level of Cbl expression and/or activity in the presence and absence of the compound wherein enhanced expression and/or activity of Cbl in the presence of the compound indicates that the compound reduces mitochondrial AMPK enzyme activity. Preferably, the AMPK activity is reduced by dephosphorylation of AMPK.

The present invention also provides a method of identifying a compound that enhances mitochondrial AMPK enzyme activity comprising determining the level of Cbl expression and/or activity in the presence and absence of the compound wherein reduced expression and/or activity of Cbl in the presence of the compound indicates that the compound enhances mitochondrial AMPK enzyme activity. Preferably, AMPK activity is enhanced by phosphorylation of AMPK.

The present invention also provides a method of identifying a compound that enhances free fatty acid synthesis comprising determining the level of Cbl expression and/or activity in the presence and absence of the compound wherein enhanced expression and/or activity of Cbl in the presence of the compound indicates that the compound enhances free fatty acid synthesis.

The present invention also provides a method of identifying a compound that reduces free fatty acid synthesis comprising determining the level of Cbl expression and/or activity in the presence and absence of the compound wherein reduced expression and/or activity of Cbl in the presence of the compound indicates that the compound reduces free fatty acid synthesis.

The present invention also provides a method of identifying a compound that reduces fatty acid oxidation comprising determining the level of Cbl expression and/or activity in the presence and absence of the compound wherein enhanced expression and/or activity of Cbl in the presence of the compound indicates that the compound reduces fatty acid oxidation.

The present invention also provides a method of identifying a compound that enhances fatty acid oxidation comprising determining the level of Cbl expression and/or activity in the presence and absence of the compound wherein reduced expression and/or activity of Cbl in the presence of the compound indicates that the compound enhances fatty acid oxidation.

The present invention also provides a method of identifying a compound that reduces expression of mitochondrial uncoupling protein 3 (UCP3) comprising determining the level of Cbl expression and/or activity in the presence and absence of the compound wherein enhanced expression and/or activity of Cbl in the presence of the compound indicates that the compound reduces UCP3 expression.

The present invention also provides a method of identifying a compound that enhances expression of mitochondrial uncoupling protein 3 (UCP3) comprising determining the level of Cbl expression and/or activity in the presence and absence of the compound wherein reduced expression and/or activity of Cbl in the presence of the compound indicates that the compound enhances UCP3 expression.

The present invention also provides a method of identifying a compound that reduces expression of an insulin receptor (IR) comprising determining the level of Cbl expression and/or activity in the presence and absence of the compound wherein enhanced expression and/or activity of Cbl in the presence of the compound indicates that the compound reduces IR expression.

The present invention also provides a method of identifying a compound that enhances expression of an insulin receptor (IR) comprising determining the level of Cbl expression and/or activity in the presence and absence of the compound wherein reduced expression and/or activity of Cbl in the presence of the compound indicates that the compound enhances IR expression.

In one embodiment, Cbl activity is determined by measuring Cbl-mediated ubiquitination of the insulin receptor in the presence and absence of the compound. Preferably, the assay comprises:
(a) providing a cell that is capable of effecting the c-Cbl-mediated ubiquitination of the insulin receptor;
(b) incubating the cell in the presence and absence of a compound to be tested; and
(c) determining c-Cbl-mediated ubiquitination of the insulin receptor in the presence and absence of the compound wherein a modified level of c-Cbl-mediated ubiquitination of the insulin receptor indicates that the compound is capable of modulating insulin action in a subject.

In one embodiment, Cbl activity is determined using an immunoassay wherein the level of c-Cbl-mediated ubiquitination of the insulin receptor is determined by contacting the insulin receptor with an antibody that binds to ubiquitin under conditions sufficient for an antigen-antibody complex to form and detecting the antibody bound to the receptor. Preferably, the assay further comprises contacting the insulin receptor with an antibody that binds to the insulin receptor under conditions sufficient for an antigen-antibody complex to form.

In an alternative embodiment, Cbl activity is determined by performing an immunoassay in a process comprising:
(a) providing a cell that is capable of effecting the c-Cbl-mediated ubiquitination of the insulin receptor;
(b) incubating the cell in the presence and absence of a compound to be tested;
(c) contacting an extract of the cell comprising the insulin receptor with an antibody that binds to the insulin receptor under conditions sufficient for an antigen-antibody complex to form thereby capturing the insulin receptor;
(d) contacting the captured insulin receptor with an antibody that binds to ubiquitin under conditions sufficient for an antigen-antibody complex to form; and
(e) detecting the antibody bound at (d).
   In accordance with this embodiment, the antibody bound at (d) is generally detected by contacting the antibody with a tertiary antibody that is capable of producing a detectable signal.

In an alternative embodiment, Cbl activity is determined in a process that measures phosphorylation of a tyrosine residue on Cbl protein in the presence and absence of the compound.

In a further embodiment, the amount of Cbl protein is determined by a process comprising:
(a) providing a cell that is capable of expressing c-Cbl protein;
(b) incubating the cell in the presence and absence of a compound to be tested; and
(c) determining amount of c-Cbl protein in the cell in the presence and absence of the compound.

In a particularly preferred embodiment, the amount of Cbl protein in a cell is determined by performing an immunoassay, e.g., wherein the amount of c-Cbl is determined by contacting the Cbl protein with an antibody that binds to Cbl under conditions sufficient for an antigen-antibody complex to form and detecting the antibody bound to the Cbl protein. Preferably, the antibody bound to the Cbl protein is detected by contacting the antibody with a secondary antibody that is capable of producing a detectable signal.

In an alternative embodiment, an immunoassay is performed wherein the amount of c-Cbl is determined by contacting the Cbl protein with a primary and secondary antibody that each bind to Cbl under conditions sufficient for antigen-antibody complexes to form and detecting an antibody bound to the Cbl protein. The antibody bound to the Cbl protein is generally detected by contacting the antibody with a secondary antibody that is capable of producing a detectable signal. The primary and secondary antibody will generally bind to different epitopes on the Cbl protein.

In a further embodiment, the amount of Cbl protein in a cell is detected by an immunoassay comprising:
(a) providing a cell that is capable of expressing c-Cbl protein;
(b) incubating the cell in the presence and absence of a compound to be tested;
(c) contacting an extract of the cell comprising the Cbl protein with an antibody that binds to Cbl protein under conditions sufficient for an antigen-antibody complex to form thereby capturing the Cbl protein; and
(d) detecting the antibody bound at (e).

In a further embodiment, the amount of Cbl protein in a cell is detected by an immunoassay comprising:
(a) providing a cell that is capable of expressing c-Cbl protein;
(b) incubating the cell in the presence and absence of a compound to be tested;
(c) contacting an extract of the cell comprising the Cbl protein with an antibody that binds to Cbl protein under conditions sufficient for an antigen-antibody complex to form thereby capturing the Cbl protein;
(d) contacting the captured Cbl protein with an antibody that binds to Cbl protein under conditions sufficient for an antigen-antibody complex to form, wherein said antibody binds to a different epitope on Cbl to the antibody at (c); and
(e) detecting the antibody bound at (d).

As with these and other preferred immunoassays, the antibody is generally detected by contacting the antibody with an antibody that is capable of producing a detectable signal.

The above-mentioned assays can be combined in any number of different permutations to test or validate a putative modulatory compound identified in cell-based or tissue-based primary screens, without undue experimentation. The present invention clearly encompasses performing assays that determine the same readout parameter (e.g., pACC, pAMPK, FFA, etc) in different physiological media (e.g., cells, tissues and intact animals), or alternatively, that determine different readout parameters in the same or different physiological media.

For animal-based assays, it is preferred that the animal subject is a non-human animal subject, preferably a mammal, such as, for example, a rodent (e.g., a rabbit, rat, guinea pig or mouse), dog, pig, bovine, sheep, horse and goat etc., and more preferably a rabbit, rat or mouse. In one embodiment, the non-human animal subject expresses an endogenous native Cbl protein (i.e. it is a wild-type animal with respect to Cbl expression). The present invention clearly provides for the use of a Cbl-knock-in animal that expresses an introduced human Cbl protein. Alternatively, a Cbl-deficient animal can be used such as, for example, a Cbl^{-/-} or Cbl^{-/+} animal, or a “Cbl knock-down animal” expressing a nucleic acid inhibitor or proteinaceous inhibitor of Cbl which partially or completely reduces Cbl expression in said animal. The present invention also contemplates the use of animals that carry or comprise a functional Cbl gene and have reduced Cbl expression by virtue of having been administered with a Cbl antagonist compound or inhibitor of Cbl expression/activity.

Preferably, a test compound is administered to muscle tissue of the animal subject. In the case of nucleic acids, it is particularly preferred that these are provided by injection. Preferably, the nucleic acids are contained within a vector such as a virus vector, in particular an adenovirus. As will be known to those skilled in the art, such adenoviruses tend to remain localized to the injection site. Accordingly, the phenotype will generally be determined in muscle tissue of the animal subject to which the vector is administered.

The various embodiments of the invention described herein for identifying compounds in animals are also suitably performed using isolated cells that have been previously derived from the animal or readily available as isolated cells, the only requirement being that the cell possesses the required Cbl phenotype to perform the assay. In one embodiment, the isolated cells are skeletal muscle cells, cardiac muscle cells, fibroblasts or fat cells (adipocytes). In lieu of using wild-type or normal animals having no Cbl deficiency, C2C12 myoblasts and/or 3T3-L1 adipocytes can be used. The various embodiments apply mutatis mutandis to the use of such isolated cells. Cell-based assays are preferably carried out using myoblasts from any animal species. In one embodiment, the cell expresses an endogenous native Cbl protein (i.e. it is a wild-type animal with respect to Cbl expression). The present invention clearly provides for the use of a Cbl-knock-in cell that consists of a non-human animal cell expressing an introduced human Cbl protein. Alternatively, a Cbl-deficient cell can be used such as, for example, a Cbl^{-/-} or Cbl^{-/+} cell, or a “Cbl knock-down cell” expressing a nucleic acid inhibitor or proteinaceous inhibitor of Cbl which partially or completely reduces Cbl expression in said cell. The present invention also contemplates the use of cells that carry or comprise a functional Cbl gene in their genome however have reduced Cbl expression by virtue of having been administered with a Cbl antagonist compound or inhibitor of Cbl expression/activity.

The present invention clearly encompasses the use of any in silico analytical method and/or industrial process for carrying the screening methods described herein into a pilot scale production or industrial scale production of a compound identified in such screens. This invention also provides for the provision of information for any such production. Accordingly, the present invention also provides a process for identifying or determining a compound or modulator supra, said method comprising:
(i) performing a method as described herein to thereby identify or determine a compound or modulator;
(ii) optionally, determining the structure of the compound or modulator; and
(iii) providing the compound or modulator or the name or structure of the compound or modulator such as, for example, in a paper form, machine-readable form, or computer-readable form.
   Naturally, for compounds that are known albeit not previously tested for their function using a screen provided by the present invention, determination of the structure of the compound is implicit in step (i) supra. This is because the skilled artisan will be aware of the name and/or structure of the compound at the time of performing the screen.

As used herein, the term “providing the compound or modulator” shall be taken to include any chemical or recombinant synthetic means for producing said compound or modulator or alternatively, the provision or a compound or modulator that has been previously synthesized by any person or means.

In a preferred embodiment, the compound or modulator or the name or structure of the compound or modulator is provided with an indication as to its use e.g., as determined by a screen described herein.

The present invention also provides a process for producing a compound or modulator supra, said method comprising:
(i) performing a method as described herein to thereby identify or determine a compound or modulator;
(ii) optionally, determining the structure of the compound or modulator;
(iii) optionally, providing the name or structure of the compound or modulator such as, for example, in a paper form, machine-readable form, or computer-readable form; and
(iv) producing or synthesizing the compound or modulator.

In a preferred embodiment, the synthesized compound or modulator or the name or structure of the compound or modulator is provided with an indication as to its use e.g., as determined by a screen described herein.

The present invention also provides methods relating to the treatment of animal or human subjects wherein a compound that modulates (i.e. enhances or reduces or prevents) ACC activity and/or AMPK activity and/or FFA level and/or fatty acid oxidation and/or Cbl expression or activity is administered to the animal. Such methods apply mutatis mutandis to the treatment of a wide range of conditions associated with aberrant insulin action, such as, for example, hyperglycemia, hyperinsulinemia, obesity, adult-onset obesity, non-insulin-dependent diabetes mellitus, type II diabetes, glucose intolerance, hypertrophy or hyperplasia of the islets of Langerhans, atherosclerosis or heart disease, or for cosmetic purposes, such as, for example, bodybuilding or weight management (i.e. weight loss or weight gain). It is to be understood that such applications of the invention also relate to the productivity of stock and farm animals (e.g. dairy and beef cattle, pigs, horses, sheep, etc) such as, for example, for modulating the amount of fat they deposit or their ratio of fat to muscle mass.

Nucleic acid inhibitors of Cbl expression, and/or genetically modified cells comprising such nucleic acid inhibitors are particularly useful for treating or preventing a condition associated with aberrant insulin action. As exemplified herein, shRNA against Cbl successfully down-regulates or silences Cbl expression in myoblasts, thereby facilitating the production of therapeutic agents that treat the onset, development, maintenance or progression of a condition associated with Cbl expression, especially conditions associated with aberrant insulin action mediated by Cbl expression. For example, ddRNAi-mediated silencing of one or more sequences known to knock out or knock down c-Cbl expression, thereby increasing phosphorylation and decreasing activity of ACC and/or or increasing phosphorylation and activity of AMPK and/or reducing free fatty acids and/or increasing fatty acid oxidation, effects control of insulin action in a subject.

Accordingly, the present invention also provides a method for treating or preventing a condition associated with aberrant insulin action in a subject, said method comprising administering to said subject a genetic construct comprising at least one ddRNAi expression cassette which encodes an RNA molecule comprising a nucleotide sequence which is at least 70% identical to at least part of a nucleotide sequence comprising a sequence that partially or completely inhibits (e.g., knocks-out or knocks-down) Cbl expression, increases phosphorylation of ACC and/or decreases activity of ACC and/or or increases phosphorylation of AMPK and/or increases activity of AMPK and/or reduces free fatty acids and/or increases fatty acid oxidation. Preferably, said encoded RNA further delays, represses or otherwise reduces the expression of the sequences that partially or completely inhibit Cbl expression, increase phosphorylation of ACC and/or decrease activity of ACC and/or or increase phosphorylation of AMPK and/or increase activity of AMPK and/or reduce free fatty acids and/or increase fatty acid oxidation.

The present invention also provides a genetically modified cell comprising a ddRNAi expression construct as described herein. Preferably the cell is a mammalian cell, even more preferably the cell is a primate or rodent cell and most preferably the cell is a human or mouse cell.

The present invention also provides a multicellular structure comprising one or more genetically modified cells of the present invention. Multicellular structures include, inter alia, include a tissue, organ or complete organism.

In accordance with this example of the invention, an amount of the compound is administered to the animal or human subject effective to modulate the expression or activity of Cbl in the subject.

### BRIEF DESCRIPTION OF THE DRAWINGS

FIG. 1 is a graphical representation showing the body weight in grams (ordinate) of male (circles) and female (triangles) wild type mice (open symbols), and Cbl-deficient male (circles) and female (triangles) mice that are homozygous for a mutation in both alleles of the Cbl locus (filled symbols). Body weights of mice were determined every week from weaning to 16 weeks of age (x-axis). Data indicate that Cbl-deficient male mice have significantly higher body weight than their wild-type counterparts, throughout the time period tested. The weights of Cbl-deficient female mice are comparable to the weights of wild-type females.

FIG. 2 is a graphical representation showing the dietary food intake in grams per day (ordinate) of male (circles) and female (triangles) wild, type mice (open symbols), and Cbl-deficient male (circles) and female (triangles) mice that are homozygous for a mutation in both alleles of the Cbl locus (filled symbols). Food intake was determined every week from 5 weeks of age until 16 weeks of age (x-axis). Data indicate that Cbl-deficient male and female mice have significantly enhanced dietary intake (i.e. enhanced appetite) than their wild-type counterparts, throughout the time period tested. The dietary intake of Cbl-deficient female mice is also significantly higher than the dietary intake of wild-type males.

FIG. 3 is a graphical representation showing the dietary food intake relative to body weight (ordinate) of male (circles) and female (triangles) wild type mice (open symbols), and Cbl-deficient male (circles) and female (triangles) mice that are homozygous for a mutation in both alleles of the Cbl locus (filled symbols). Food intake relative to body weight was determined every week from 5 weeks of age until 16 weeks of age (x-axis). Data indicate that Cbl-deficient male and female mice have significantly enhanced specific dietary intake (i.e. enhanced appetite as determined by food intake relative to body weight) than their wild-type counterparts, throughout the time period tested.

FIG. 4 is a tabular representation showing the tissue weights (average +/−SEM) of wild type mice and Cbl-deficient mice that are homozygous for a mutation in both alleles of the Cbl locus (filled symbols). WAT, white adipose tissue; BAT, brown adipose tissue; QUAD, quadriceps muscle. Data indicate reduced adipose tissue weight for Cbl-deficient animals relative to wild type animals.

FIG. 5 is a photographic representation showing adipocyte size in wild type mice (left) and Cbl-deficient mice that are homozygous for a mutation in both alleles of the Cbl locus (right).

FIG. 6 is a tabular representation showing adipocyte diameter (μm) adipocyte volume (pl/cell) and lipid content (ng/cell) of male (Top panel) and female (Lower panel) wild type mice (WT), compared to male (Top panel) and female (Lower panel) Cbl-deficient mice that are homozygous for a mutation in both alleles of the Cbl locus (KO). Data indicate the mean values +/−SEM. Data indicate that for both males and females, there is a significant reduction in adipocyte size and volume, and reduced lipid content of adipocytes in Cbl-deficient mice relative to wild-type mice.

FIG. 7a is a graphical representation showing glucose transport in soleus muscles of c-CBL^{-/-} mice. Soleus muscles were removed from c-CBL−/− mice and wild-type c-CBL+/+ mice and incubated in the presence of labeled 2-deoxyglucose and no insulin (basal), 300 μU/ml insulin (submax) and 1000 μU/ml insulin (supramax). The muscle was then liquefied and the amount of labeled 2-deoxyglucose taken up by the muscle determined using a liquid scintillation counter. This amount +/−SEM was then graphically represented. *, p<0.05; *, p<0.01.

FIG. 7b is a graphical representation showing glucose transport in extensor digitorum longus muscles of c-CBL^{-/-} mice. Extensor digitorum longus muscles were removed from c-CBL−/− mice and wild-type c-CBL+/+ mice and incubated in the presence of labeled 2-deoxyglucose and no insulin (basal), 300 μU/ml insulin (submax) and 1000 μU/ml insulin (supramax). The muscle was then liquefied and the amount of labeled 2-deoxyglucose taken up by the muscle determined using a liquid scintillation counter. This amount +/−SEM was then graphically represented. *, p<0.05.

FIG. 7c is a graphical representation showing glucose transport in fat explants taken from c-CBL^{-/-} mice. Epididymal fat pads removed from c-CBL−/− mice and wild-type c-CBL+/+ mice and minced. Samples were then incubated in the presence of labeled 2-deoxyglucose and no insulin (0), 0.05 nM insulin (0.05) and 1 nM insulin (1). The amount of labeled 2-deoxyglucose taken up by the fat explant was determined using a liquid scintillation counter. This amount +/−SEM was then graphically represented.

FIG. 8a provides graphical representations showing blood glucose levels in overnight-fasted wild-type (WT; filled circles/bars, +/+) and c-Cbl-deficient mice (open circles/bars, −/−) during the course of a glucose tolerance test (2 g/kg) in 16- to 18-week-old mice. Left, time course of blood glucose levels. Right, area under each curve shown on left. Data represent the mean ±SEM of 10-12 animals per group.

FIG. 8b provides graphical representations showing plasma insulin levels in overnight-fasted wild-type (WT; filled circles/bars, +/+) and c-Cbl-deficient mice (open circles/bars, −/−) during the course of a glucose tolerance test (2 g/kg) in 16- to 18-week-old mice. Left, time course of insulin levels. Right, area under each curve shown on left. Data represent the mean ±SEM of 10-12 animals per group.

FIG. 8c provides graphical representations showing percentage change in blood glucose in overnight-fasted wild-type (WT; filled circles/bars, +/+) and c-Cbl-deficient mice (open circles/bars, −/−) during the course of a glucose tolerance test (2 g/kg) in 16- to 18-week-old mice. Left, time course of blood glucose expressed as a percentage of basal blood glucose. Right, area under each curve shown on left. Data represent the mean ±SEM of 10-12 animals per group.

FIG. 8d provides a graphical representation showing clearance of the glucose analogue [³H] 2-deoxyglucose into glucose-6-phosphate in muscle (M), brown adipose tissue (B) and white adipose tissue (W) during a glucose tolerance test on overnight-fasted wild-type (WT; filled bars, +/+) and Cbl-deficient mice (open bars, −/−) during the course of a glucose tolerance test (2 g/kg) in 16- to 18-week-old mice. Data represent the mean ±SEM of 5 animals per group.

FIG. 8e provides a graphical representation showing clearance of the glucose analogue [³H] 2-deoxyglucose into glycogen in muscle (M) and liver (L) during a glucose tolerance test on overnight-fasted wild-type (WT; filled bars, +/+) and Cbl-deficient mice (open bars, −/−) during the course of a glucose tolerance test (2 g/kg) in 16- to 18 week-old mice. Data represent the mean ±SEM of 5 animals per group.

FIG. 9 provides copies of photographic representations showing insulin receptor (IR) protein levels in insulin sensitive tissues of wild-type (+/+) and Cbl-deficient (−/−) animals. Aliquots (5 and 15 μg total protein) of quadriceps (top rows, Muscle), epididymal white adipose tissue (middle row, WAT) and liver (bottom rows, Liver) homogenates from WT and c-Cbl-deficient mice were resolved by SDS-PAGE and immunoblotted with antibodies specific for the β subunit of the IR (βIR) and syntaxin4 (SYN4). The fold increase in protein levels observed in c-Cbl^{-/-} mice compared with WT animals, as well as the result of the statistical analysis of the data, is shown at the right of each row. These values represent the average of 6-12 animals in each group.

FIG. 10a is a graphical representation showing the amount of phosphorylated ACC in Cbl−/− mice. Protein derived from quadriceps of Cbl−/− (KO) and wild-type (WT) mice was resolved using SDS-PAGE and transferred to a membrane. The amount of phosphorylated ACC was then determined using an anti-phosphorylated ACC antibody. Results are presented as percentage of phosphorylated ACC detected in wild-type mice. *, p<0.05.

FIG. 10b is a graphical representation showing the amount of AMP activated protein kinase (AMPK) in Cbl−/− mice. Protein derived from quadriceps of Cbl−/− (KO) and wild-type (WT) mice was resolved using SDS-PAGE and transferred to a membrane. The amount of AMPK was then determined using an anti-AMPK antibody. Results are presented as percentage of AMPK detected in wild-type mice.

FIG. 10c is a graphical representation showing the amount of cytochrome-C (CytC) in Cbl−/− mice. Protein derived from quadriceps of Cbl−/− (KO) and wild-type (WT) mice was resolved using SDS-PAGE and transferred to a membrane. The amount of CytC was then determined using an anti-CytC antibody. Results are presented as percentage of CytC detected in wild-type mice. *, p<0.05.

FIG. 10d is a graphical representation showing the amount of uncoupling protein 3 (UCP3) in Cbl−/− mice. Protein derived from quadriceps of Cbl−/− (KO) and wild-type (WT) mice was resolved using SDS-PAGE and transferred to a membrane. The amount of UCP3 was then determined using an anti-UCP3 antibody. Results are presented as percentage of UCP3 detected in wild-type mice. *, p<0.05.

FIG. 10e is a graphical representation showing the amount of insulin receptor (IR) in Cbl−/− mice. Protein derived from quadriceps of Cbl−/− (KO) and wild-type (WT) mice was resolved using SDS-PAGE and transferred to a membrane. The amount of IR was then determined using an anti-IR antibody. Results are presented as percentage of IR detected in wild-type mice. *, p<0.05.

FIG. 10f is a graphical representation showing the amount of glucose transporter 4 (GLUT4) in Cbl−/− mice. Protein derived from quadriceps of Cbl−/− (KO) and wild-type (WT) mice was resolved using SDS-PAGE and transferred to a membrane. The amount of GLUT4 was then determined using an anti-GLUT4 antibody. Results are presented as percentage of GLUT4 detected in wild-type mice. *, p<0.05.

FIG. 11a provides copies of representative electron micrographs of soleus muscle from wild-type (+/+) and c-Cbl-deficient (−/−) mice showing the subsarcolemmal mitochondria population. Scale bar: 1 μm. The histogram shows the average mitochondrial size from more than 100 mitochondria from matching sections of muscles from wild-type mice (filled bars) and c-Cbl-deficient mice (open bars).

FIG. 11b provides copies of photographic representations showing levels of mitochondrial proteins PGC-1α, UCP3, phosphorylated ACC (pACC), and total ACC in Cbl-deficient mice (−/−) and wild-type (+/+) mice. Muscle homogenates were immunoblotted with either antibodies specific for UCP3, pSer79-ACC, ACC, and PGC1α (5 and 15 μg total protein). The fold increase in protein levels observed in c-Cbl^{-/-} mice compared with wild-type animals, as well as the result of the statistical analysis of the data, is shown at the right of each lane. Data represent the mean ±SEM of 12 animals in each group.

FIG. 11c provides copies of photographic representations showing levels of AMPK (AMKα) and phosphorylated AMPK (pAMKα) in Cbl-deficient mice (−/−) and wild-type (+/+) mice. Muscle homogenates were immunoblotted with either antibodies specific for AMPK and pThr172-AMPK (60 μg total protein). The fold increase in protein levels observed in c-Cbl^{-/-} mice compared with wild-type animals, as well as the result of the statistical analysis of the data, is shown at the right of each lane. Data represent the mean ±SEM of 6 animals in each group.

FIG. 11d is a graphical representation showing ACC phosphorylation as a ratio of total ACC (top) and AMPK phosphorylation as a ratio of total AMPK (bottom) in quadriceps from wild-type and c-Cbl^{-/-} mice. *P<0.05, **P<0.01.

FIG. 12a is a copy of a photographic representation showing shRNA knockdown of c-Cbl protein expression in murine C2C12 myoblasts. Cells were transfected using lipofectin, with either no DNA (left column marked “lipof”), or 15 mg of control vector DNA comprising shRNA targeting green fluorescent protein (GFP) expression (middle column marked “GFP”) or 15 mg of vector DNA comprising shRNA targeting c-Cbl expression (right column marked “c-Cbl”). After 3 days, cells were homogenized and western blots performed using antibodies against c-Cbl. Data show reduced expression of c-Cbl only in the presence of shRNA against c-Cbl.

FIG. 12b is a graphical representation showing expression levels of c-Cbl, Cbl-b, insulin receptor (IR) and the mitochondrial proteins AMPK (tAMPK) and ACC, and the phosphorylated forms thereof, pAMPK and pACC. Data are presented as the percentage expression of each protein in the presence of shRNA targeting c-Cbl expression relative to the expression level of that protein in the presence of shRNA targeting GFP expression (FIG. 12A). The ratio of total AMPK to phosphorylated AMPK (p/tAMPK) was also determined. Data are presented as mean ±SEM for 2-4 samples in each group. Data indicate that targetted knockdown of c-Cbl expression does not affect expression of Cbl-b or total AMPK level, but leads to a 5-fold increase in IR levels, and a 3-fold increase in pAMPK and 2-fold increase in pACC, thereby mimicking the phenotype seen in c-Cbl^{-/-} knockout animals.

FIG. 13 is a schematic representation of a method for delivering RNAi agents to according to the present invention.

FIG. 14a is a schematic representation of a first single-expression RNAi cassette.

FIG. 14b is a schematic representation of a second single-expression RNAi cassette.

FIG. 14c is a schematic representation of a first multiple-expression RNAi cassette.

FIG. 14d is a schematic representation of a second multiple-expression RNAi cassette.

FIG. 15a is a schematic representation of a first multiple expression cassette encoding an RNAi agent initially expressed as a shRNA precursor.

FIG. 15b is a schematic representation of a second multiple expression cassette encoding an RNAi agent initially expressed as a shRNA precursor.

FIG. 15c is a schematic representation of a first multiple expression cassette encoding an RNAi agent that is not expressed as a shRNA precursor.

FIG. 15d is a schematic representation of a second multiple expression cassette encoding an RNAi agent initially expressed as a shRNA precursor.

FIG. 16a is a schematic representation showing a first method for producing viral particles for delivery of a ddRNAi agent to appropriate target tissue.

FIG. 16b is a schematic representation showing a second method for producing viral particles for delivery of a ddRNAi agent to appropriate target tissue.

### DETAILED DESCRIPTION OF THE PREFERRED EMBODIMENTS

Modulatory Compounds and Formulations Generally

The range of compounds contemplated herein for testing include peptides, e.g., peptides derived from Cbl and capable of complementing a Cbl-deficiency; non-Cbl peptides, such as Cbl peptidomimetics; small organic molecules, such as, for example derived from commercial sources, or publicly available combinatorial libraries; and nucleic acids, including nucleic acid encoding a non-Cbl peptide.

The compounds contemplated also include Cbl inhibitory compounds or antagonists of a biological function of Cbl. In one embodiment, the compound is selected from the group consisting of: a peptide, including a peptide derived from Cbl and capable of inhibiting, reducing or repressing a Cbl function, including binding to a protein selected from the group consisting of CAP, CrkII and C3G; a Cbl dominant-negative mutant; a non-Cbl peptide inhibitors of Cbl; an antibody or antibody fragment which binds to Cbl and inhibits a Cbl function; a small organic molecule, and nucleic acid, including nucleic acid encoding said peptide derived from Cbl or said non-Cbl peptide inhibitor, an antisense nucleic acid directed against Cbl-encoding mRNA, or an anti-Cbl ribozyme, or a small interfering RNA (RNAi) that targets Cbl gene expression.

The term “antisense nucleic acid” shall be taken to mean DNA or RNA molecule that is complementary to at least a portion of a specific mRNA molecule (Weintraub, Scientific American 262:40, 1990) and capable of interfering with a post-transcriptional event such as mRNA translation. Antisense oligomers complementary to at least about 15 contiguous nucleotides of Cbl-encoding mRNA are preferred, since they are easily synthesized and are less likely to cause problems than larger molecules when introduced into the target Cbl-producing cell. The use of antisense methods is well known in the art (Marcus-Sakura, Anal. Biochem. 172: 289, 1988). Preferred antisense nucleic acid will comprise a nucleotide sequence that is complementary to at least 15 contiguous nucleotides of a sequence encoding the amino acid sequence set forth in SEQ ID NO: 2 or 3.

As used herein, a “ribozyme” is a nucleic acid molecule having nuclease activity for a specific nucleic acid sequence. A ribozyme specific for Cbl-encoding mRNA, for example, binds to and cleaves specific regions of the mRNA, thereby rendering it untranslatable. To achieve specificity, preferred ribozymes will comprise a nucleotide sequence that is complementary to at least about 12-15 contiguous nucleotides of a sequence encoding the amino acid sequence set forth in SEQ ID NO: 2 or 3.

As used herein, the terms “small interfering RNA” (“siRNA”), short hairpin RNA (“shRNA”), and “RNAi” refer to homologous double stranded RNA (dsRNA) that specifically targets a gene product, thereby resulting in a null or hypomorphic phenotype. Specifically, the dsRNA comprises two short nucleotide sequences derived from the target RNA encoding Cbl and having self-complementarity such that they can anneal, and interfere with expression of a target gene, presumably at the post-transcriptional level. RNAi molecules are described by Fire et al., Nature 391, 806-811, 1998, and reviewed by Sharp, Genes & Development, 13, 139-141, 1999). As will be known to those skilled in the art, short hairpin RNA (“shRNA”) is similar to siRNA, however comprises a single strand of nucleic acid wherein the complementary sequences are separated an intervening hairpin loop such that, following introduction to a cell, it is processed by cleavage of the hairpin loop into siRNA. Accordingly, each and every embodiment described herein is equally applicable to siRNA and shRNA.

Specifically, the term “RNA agent” refers to an RNA sequence that elicits RNAi; and the term “ddRNAi agent” refers to an RNAi agent that is transcribed from a vector. The terms “short hairpin RNA” or “shRNA” refer to an RNA structure having a duplex region and a loop region. In some embodiments of the present invention, ddRNAi agents are expressed initially as shRNAs. The term “RNAi expression cassette” refers to a cassette according to embodiments of the present invention having at least one [promoter-RNAi agent-terminator] unit. The term “multiple promoter RNAi expression cassette” refers to an RNAi expression cassette comprising two or more [promoter-RNAi agent-terminator] units. The terms “RNAi expression construct” or “RNAi expression vector” refer to vectors containing an RNAi expression cassette.

The present invention clearly encompasses methods for producing or synthesizing a compound that is tested in accordance with the invention.

Peptidyl Compounds

Peptidyl compounds are conveniently made by standard peptide synthesis, such as the Merrifield method of synthesis (Merrifield, J Am Chem Soc, 85, 2149-2154, 1963) and the myriad of available improvements on that technology (see e.g., Synthetic Peptides: A User's Guide, Grant, ed. (1992) W.H. Freeman & Co., New York, pp. 382; Jones (1994) The Chemical Synthesis of Peptides, Clarendon Press, Oxford, pp. 230.); Barany, G. and Merrifield, R. B. (1979) in The Peptides (Gross, E. and Meienhofer, J. eds.), vol. 2, pp. 1-284, Academic Press, New York; Wunsch, E., ed. (1974) Synthese von Peptiden in Houben-Weyls Metoden der Organischen Chemie (Müler, E., ed.), vol. 15, 4th edn., Parts 1 and 2, Thieme, Stuttgart; Bodanszky, M. (1984) Principles of Peptide Synthesis, Springer-Verlag, Heidelberg; Bodanszky, M. & Bodanszky, A. (1984) The Practice of Peptide Synthesis, Springer-Verlag, Heidelberg; Bodanszky, M. (1985) Int. J. Peptide Protein Res. 25, 449-474.

Preferably, the peptide is synthesized on a solid phase support, such as, for example, a polystyrene gel bead comprising polystyrene cross-linked with divinylbenzene, preferably 1% (w.w) divinylbenzene, which is further swollen using lipophilic solvent, such as, for example dichloromethane or dimethylformamide (DMF). The polystyrene can be functonalized by addition of chloromethane or amino methyl groups. Alternatively, cross-linked and functionalized polydimethyl-acrylamide gel can be used once swollen and solvated using DMF or dipolar aprotic solvent. Other solid phase supports known to those skilled in the art can also be used for peptide synthesis, such as, for example, polyethylene glycol-derived bead produced by grafting polyethylene glycol to the surface of inert polystyrene beads. Preferred commercially available solid phase supports include PAL-PEG-PS, PAC-PEG-PS, KA, KR, or TGR (Applied Biosystems, CA 94404, USA).

For solid phase peptide synthesis, blocking groups that are stable to the repeated treatments necessary for removal of the amino blocking group of the growing peptide chain and for repeated amino acid couplings, are used for protecting the amino acid side-chains during synthesis and for masking undesired reactivity of the α-amino, carboxyl or side chain functional groups. Blocking groups (also called protecting groups or masking groups) thus protect the amino group of the amino acid having an activated carboxyl group that is involved in the coupling reaction, or protect the carboxyl group of the amino acid having an acylated amino group that is involved in the coupling reaction.

During synthesis, coupling occurs following removal of a blocking group without the disruption of a peptide bond, or any protecting group attached to another part of the peptide. Additionally, the peptide-resin anchorage that protects the C-terminus of the peptide is protected throughout the synthetic process until cleavage from the resin is required. Accordingly, by the judicious selection of orthogonally protected α-amino acids, amino acids are added at desired locations to a growing peptide whilst it is still attached to the resin.

Preferred amino blocking groups are easily removable but sufficiently stable to survive conditions for the coupling reaction and other manipulations, such as, for example, modifications to the side-chain groups. In one embodiment, amino blocking groups are selected from the group consisting of: (i) a benzyloxycarbonyl group (Z or carbocenzoxy) that is removed easily by catalytic hydrogenation at room temperature and ordinary pressure, or using sodium in liquid ammonia and hydrobromic acid in acetic acid; (ii) a urethane derivative; (iii) a t-Butoxycarbonyl group (Boc) that is introduced using t-butoxycarbonyl azide or di-tert-butyldicarbonate and removed using mild acid such as, for example, trifluoroacetic acid (50% TFA in dichloromethane), or HCl in acetic acid/dioxane/ethylacetate; (iv) a 9-fluorenylmethyloxycarbonyl group (Fmoc) that is cleaved under mildly basic, non-hydrolytic conditions, such as, for example, using a primary or secondary amine (eg. 20% piperidine in dimethyl formamide); (v) a 2-(4-biphenylyl)propyl(2)oxycarbonyl group (Bpoc); (vi) a 2-nitro-phenylsulfenyl group (Nps); and (vii) a dithia-succionyl group (Dts). Boc is widely used to protect the N-terminus in Fmoc chemistry, or Fmoc is widely used to protect the N-terminus in Boc chemistry.

Side chain-protecting groups will vary for the functional side chains of the amino acids forming the peptide being synthesized. Side-chain protecting groups are generally based on the Bzl group or the tBu group. Amino acids having alcohols or carboxylic acids in the side-chain are protected as Bzl ethers, Bzl esters, cHex esters, tBu ethers, or tBu esters. Side-chain protection of Fmoc amino acids requires blocking groups that are ideally base stable and weak acid (TFA) labile. Many different protecting groups for peptide synthesis have been described (see The Peptides, Gross et al., eds., Vol. 3, Academic Press, New York, 1981). For example, the 4-methoxy-2,3,6-trimethylphenylsulfonyl (Nd-Mtr) group is useful for Arginine side-chain protection, however deprotection of Arg(Mtr) requires prolonged TFA treatment. A number of soft acid (TFA, thalium (III) trifluoroacetate/TFA) labile groups, or TFA stable but thalium (III) trifluoroacetate/TFA labile groups, or soft acid stable groups are used to protect Cystine.

The two most widely used protection strategies are the Boc/Bzl- and the Fmoc/tBu-strategies. In Boc/Bzl, Boc is used for amino protection and the side-chains of the various amino acids are protected using Bzl- or cHex-based protecting groups. A Boc group is stable under catalytic hydrogenation conditions and is used orthogonally along with a Z group for protection of many side chain groups. In Fmoc/tBu, Fmoc is used for amino protection and the side-chains are protected with tBu-based protecting groups.

Alternatively, the peptidyl compound is produced by the recombinant expression of nucleic acid encoding the amino acid sequence of said peptide. Random peptide-encoding libraries are particularly preferred for such purposes, because they provide a wide range of different compounds to test. Alternatively, naturally-occurring nucleic acids can be screened. According to this embodiment, nucleic acid encoding the peptidyl compound is produced by standard oligonucleotide synthesis or derived from a natural source and cloned into a suitable expression vector in operable connection with a promoter or other regulatory sequence capable of regulating expression in a cell-free system or cellular system.

Oligonucleotides are preferably synthesized with linker or adaptor sequences at the 5′- and 3′-ends to facilitate subsequent cloning into a suitable vector system using standard techniques.

Placing a nucleic acid molecule under the regulatory control of, i.e., “in operable connection with”, a promoter sequence means positioning said molecule such that expression is controlled by the promoter sequence, generally by positioning the promoter 5′ (upstream) of the peptide-encoding sequence.

The prerequisite for producing intact peptides in bacteria such as E. coli is the use of a strong promoter with an effective ribosome binding site. Typical promoters suitable for expression in bacterial cells such as E. coli include, but are not limited to, the lacz promoter, temperature-sensitive λ_{L} or λ_{R} promoters, T7 promoter or the IPTG-inducible tac promoter. A number of other vector systems for expressing the nucleic acid molecule of the invention in E. coli are well-known in the art and are described, for example, in Ausubel et al (In: Current Protocols in Molecular Biology. Wiley Interscience, ISBN 047150338, 1987) or Sambrook et al (In: Molecular cloning, A laboratory manual, second edition, Cold Spring Harbor Laboratory, Cold Spring Harbor, N.Y., 1989). Numerous plasmids with suitable promoter sequences for expression in bacteria and efficient ribosome binding sites have been described, such as for example, pKC30 (λ_{L}: Shimatake and Rosenberg, Nature 292, 128, 1981); pKK173-3 (tac: Amann and Brosius, Gene 40, 183, 1985), pET-3 (T7: Studier and Moffat, J. Mol. Biol. 189, 113, 1986); the pBAD/TOPO or pBAD/Thio-TOPO series of vectors containing an arabinose-inducible promoter (Invitrogen, Carlsbad, Calif.), the latter of which is designed to also produce fusion proteins with thioredoxin to enhance solubility of the expressed protein; the pFLEX series of expression vectors (Pfizer Inc., CT, USA); or the pQE series of expression vectors (Qiagen, CA), amongst others.

Typical promoters suitable for expression in viruses of eukaryotic cells and eukaryotic cells include the SV40 late promoter, SV40 early promoter and cytomegalovirus (CMV) promoter, CMV-IE (cytomegalovirus immediate early) promoter, H1-RNA promoter, and U6 small nuclear RNA promoter, amongst others. Preferred vectors for expression in mammalian cells (eg., HeLa cells, HUVEC cells, 293 cells, 293 T cells, COS cells, COS-1 cells, CHO cells, CHO.T cells, C2C12 cells, differentiated 3T3-L1 adipocytes, 10T cells) include, but are not limited to, the pcDNA vector suite supplied by Invitrogen, in particular pcDNA 3.1 myc-His-tag comprising the CMV promoter and encoding a C-terminal 6×His and MYC tag; and the retrovirus vector pSRαtkneo (Muller et al., Mol. Cell. Biol., 11, 1785, 1991). The vector pcDNA 3.1 myc-His (Invitrogen) is particularly preferred for expressing peptides in a secreted form in 293T cells, wherein the expressed peptide or protein can be purified free of conspecific proteins, using standard affinity techniques that employ a Nickel column to bind the protein via the His tag.

A wide range of additional host/vector systems suitable for expressing peptides are available publicly, and described, for example, in Sambrook et al (In: Molecular cloning, A laboratory manual, second edition, Cold Spring Harbor Laboratory, Cold Spring Harbor, N.Y., 1989).

Means for introducing the nucleic acid or a gene construct comprising same into a cell for expression are well-known to those skilled in the art. The technique used for a given organism depends on the known successful techniques. Means for introducing recombinant DNA into animal cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

In one embodiment, peptidyl Cbl inhibitors are chemically or recombinantly-synthesized as oligopeptides (approximately 10-25 amino acids in length) spanning the Cbl sequence (SEQ ID NO: 2 or 3). Alternatively, Cbl fragments are produced by digestion of native or recombinantly produced Cbl by, for example, using a protease, e.g., trypsin, thermolysin, chymotrypsin, or pepsin. Computer analysis (using commercially available software, e.g. MacVector, Omega, PCGene, Molecular Simulation, Inc.) is used to identify proteolytic cleavage sites. The proteolytic or synthetic fragments can comprise as many amino acid residues as are necessary to partially or completely inhibit Cbl function. Preferred fragments will comprise at least 5, 10, 15, 20, 25, 30, 35, 40, 45, 50, 55, 60, 65, 70, 75, 80, 85, 90, 95, 100 or more amino acids in length.

In one embodiment, peptides are selected which contain a sufficient number of B cell epitopes to elicit antibodies when administered to a mammal. Such peptides are identified by immunizing a mammal with the peptide alone, or in combination with an adjuvant, or linked to an adjuvant (e.g., a hapten). Sera from the immunized animal are tested for anti-Cbl antibodies. Preferred peptides generate anti-Cbl antibodies that inhibit a Cbl function. Preferred peptidyl Cbl inhibitors will also not comprise a sufficient number of T cell epitopes to induce T-cell mediated (e.g., cytokine) responses when determined using any of a number of well known techniques, such as epitope prediction using algorithms (see e.g., Rothbard and Taylor EMBO J. 7: 93-100, 1988; Berzofsky, Philos Trans R. Soc. Lond. 323: 535-544, 1989; Rothbard, 1st Forum in Virology, Annals of the Pasteur Institute, (pp 518-526, December 1986; Rothbard and Taylor, Embo, 7: 93-100, 1988; EP 0 304 279; and Margalit et al., J. Immunol., 138: 2213-2229, 1987); or screening of peptide inhibitors for human T cell stimulating activity or T cell proliferation assays (e.g. Proc. Natl. Acad. Sci. USA, 86:1333, 1989).

Other preferred peptide inhibitors of Cbl are located on the surface of the Cbl proteins, e.g., hydrophilic regions, as well as regions with high antigenicity or fragments with high surface probability scores can be identified using computer analysis programs well known to those of skill in the art (Hopp and Wood, (1983), Mol. Immunol., 20, 483-9, Kyte and Doolittle, (1982), J. Mol. Biol., 157, 105-32, Corrigan and Huang, (1982), Comput. Programs Biomed, 3, 163-8).

Small Organic Compounds

Techniques for synthesizing small organic compounds will vary considerably depending upon the compound, however such methods will be well known to those skilled in the art. In one embodiment, informatics is used to select suitable chemical building blocks from known compounds, for producing a combinatorial library. For example, QSAR (Quantitative Structure Activity Relationship) modelling approach uses linear regressions or regression trees of compound structures to determine suitability. The software of the Chemical Computing Group, Inc. (Montreal, Canada) uses high-throughput screening experimental data on active as well as inactive compounds, to create a probabilistic QSAR model, which is subsequently used to select lead compounds. The Binary QSAR method is based upon three characteristic properties of compounds that form a “descriptor” of the likelihood that a particular compound will or will not perform a required function: partial charge, molar refractivity (bonding interactions), and logP (lipophilicity of molecule). Each atom has a surface area in the molecule and it has these three properties associated with it. All atoms of a compound having a partial charge in a certain range are determined and the surface areas (Van der Walls Surface Area descriptor) are summed. The binary QSAR models are then used to make activity models or ADMET models, which are used to build a combinatorial library. Accordingly, information from known appetite suppressants and non-suppressants, including lead compounds identified in initial screens, can be used to expand the list of compounds being screened to thereby identify highly active compounds.

In one embodiment, the present invention a small organic molecule inhibitor or antagonist of c-Cbl is a compound that reduces the E3 ubiquitin ligase activity of c-Cbl thereby reduces the ubiquitination of the insulin receptor β-subunit by c-Cbl. Preferably, the small organic molecule is a non-competitive inhibitor of c-Cbl with respect to ubiquitin and the insulin receptor β-subunit. In a preferred embodiment, the small organic molecule belongs to a class of compounds that binds to the RING finger domain of c-Cbl (i.e. an amino acid sequence comprising or contained within residues from about position 380 to about position 421 of SEQ ID NO: 3), e.g., a benzsulfonamide, urea, or imidazolone.

RNAi Inhibitors of Cbl Expression

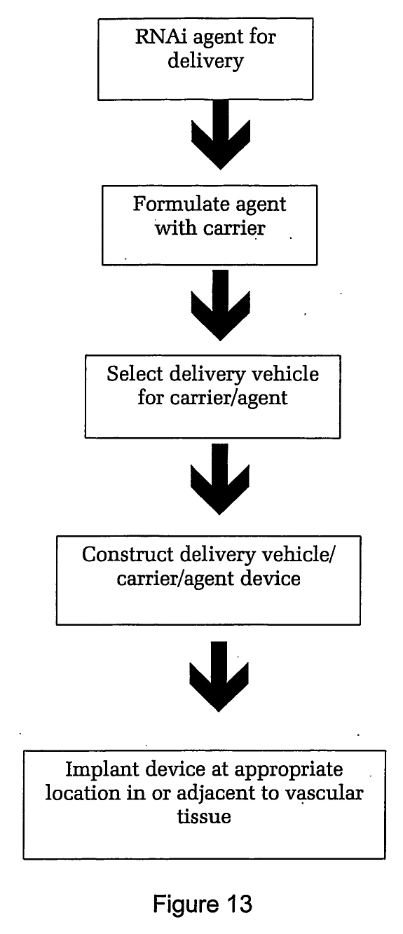

FIGS. 13a through 13c are simplified flow charts showing the process steps in which an RNAi agent according to the present invention may be used, including a step in which a ddRNAi expression cassette is constructed. Such a ddRNAi expression cassette most often will include at least one promoter, a ddRNAi sequence to be expressed, and at least one terminator. Various configurations of such ddRNAi expression cassettes are described in detail infra.

As described in FIG. 13a, the ddRNAi expression cassette is ligated into viral delivery vector, and the ddRNAi viral delivery vector is packaged into viral particles. Finally, viral particles are delivered to target cells, tissues or organs. FIG. 13b shows a method where a ddRNAi expression cassette is constructed., wherein the ddRNAi expression cassette is ligated into a non-viral delivery vector which is then delivered to target cells, tissues or organs. FIG. 13c shows a method wherein an siRNA agent is constructed for delivery, such that the siRNA is formulated with an appropriate carrier for delivery and the siRNA agent/carrier is delivered to target cells, tissues, or organs.

RNAi agents according to the present invention are generated synthetically or enzymatically by a number of different protocols known to those skilled in the art and purified using standard recombinant DNA techniques as described in, for example, Sambrook et al., Molecular Cloning: A Laboratory Manual, 2nd Ed., Cold Spring Harbor Press, Cold Spring Harbor, N.Y. (1989), and under regulations described in, e.g., United States Dept. of HHS, National Institute of Health (NIH) Guidelines for Recombinant DNA Research.

RNAi agents may comprise either siRNAs (synthetic RNAs) or DNA-directed RNAs (ddRNAs). siRNAs may be manufactured by methods known in the art such as by typical oligonucleotide synthesis, and often will incorporate chemical modifications to increase half life and/or efficacy of the siRNA agent, and/or to allow for a more robust delivery formulation. Many modifications of oligonucleotides are known in the art. For example, U.S. Ser. No. 6,620,805 discloses an oligonucleotide that is combined with a macrocycle having a net positive charge such as a porphyrin; U.S. Ser. No. 6,673,611 discloses various formulas; U.S. Pub. Nos. 2004/0171570, 2004/0171032, and 2004/0171031 disclose oligomers that include a modification comprising a polycyclic sugar surrogate; such as a cyclobutyl nucleoside, cyclopentyl nucleoside, proline nucleoside, cyclohexene nucleoside, hexose nucleoside or a cyclohexane nucleoside; and oligomers that include a non-phosphorous-containing internucleoside linkage; U.S. Pub. No 2004/0171579 discloses a modified oligonucleotide where the modification is a 2′ substituent group on a sugar moiety that is not H or OH; U.S. Pub. No. 2004/0171030 discloses a modified base for binding to a cytosine, uracil, or thymine base in the opposite strand comprising a boronated C and U or T modified binding base having a boron-containing substituent selected from the group consisting of —BH2CN, —BH3, and —BH2COOR, wherein R is C1 to C18 alkyl; U.S. Pub. No. 2004/0161844 discloses oligonucleotides having phosphoramidate internucleoside linkages such as a 3′aminophosphoramidate, aminoalkylphosphoramidate, or aminoalkylphosphorthioamidate internucleoside linkage; U.S. Pub. No. 2004/0161844 discloses yet other modified sugar and/or backbone modifications, where in some embodiments, the modification is a peptide nucleic acid, a peptide nucleic acid mimic, a morpholino nucleic acid, hexose sugar with an amide linkage, cyclohexenyl nucleic acid (CeNA), or an acyclic backbone moiety; U.S. Pub. No. 2004/0161777 discloses oligonucleotides with a 3′ terminal cap group; U.S. Pub. No. 2004/0147470 discloses oligomeric compounds that include one or more cross-linkages that improve nuclease resistance or modify or enhance the pharmacokinetic and pharmacodynamic properties of the oligomeric compound where such cross-linkages comprise a disulfide, amide, amine, oxime, oxyamine, oxyimine, morpholino, thioether, urea, thiourea, or sulfonamide moiety; U.S. Pub. No. 2004147023 discloses a gapmer comprising two terminal RNA segments having nucleotides of a first type and an internal RNA segment having nucleotides of a second type where nucleotides of said first type independently include at least one sugar substituent where the sugar substituent comprises a halogen, amino, trifluoroalkyl, trifluoroalkoxy, azido, aminooxy, alkyl, alkenyl, alkynyl, O-, S-, or N(R*)-alkyl; O-, S-, or N(R*)-alkenyl; O-, S- or N(R*)-alkynyl; O-, S- or N-aryl, O-, S-, or N(R*)-aralkyl group; where the alkyl, alkenyl, alkynyl, aryl or aralkyl may be a substituted or unsubstituted alkyl, alkenyl, alkynyl, aralkyl; and where, if substituted, the substitution is an alkoxy, thioalkoxy, phthalimido, halogen, amino, keto, carboxyl, nitro, nitroso, cyano, trifluoromethyl, trifluoromethoxy, imidazole, azido, hydrazino, aminooxy, isocyanato, sulfoxide, sulfone, disulfide, silyl, heterocycle, or carbocycle group, or an intercalator, reporter group, conjugate, polyamine, polyamide, polyalkylene glycol, or a polyether of the formula (—O-alkyl)m, where m is 1 to about 10; and R* is hydrogen, or a protecting group; or U.S. Pub. No. 2004/0147022 disclosing an oligonucleotide with a modified sugar and/or backbone modification, such as a 2-OCH3 substituent group on a sugar moiety.

Alternatively, DNA-directed RNAi (ddRNAi) agents may be employed. ddRNAi agents comprise an expression cassette, most often containing at least one promoter, at least one ddRNAi sequence and at least one terminator in a viral or non-viral vector backbone. For example, in one preferred embodiment, the ddRNAi expression cassette comprises a nucleic acid molecule comprising the general structure (I): 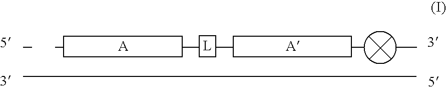
wherein:
represents a promoter sequence; 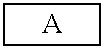
represents a ddRNAi targeting sequence comprising at least 10 nucleotides, wherein said sequence is at least 70% identical to a portion of a Cbl gene, including a sequence known to knock out or knock down c-Cbl and/or increase phosphorylation of ACC and/or decrease activity of ACC and/or increase phosphorylation of AMPK and/or increase activity of AMPK and/or increase fatty acid oxidation and/or decrease free fatty acids in a cell; 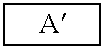
represents a sequence of 10 to 30 nucleotides wherein at least 10 contiguous nucleotides of A′ comprise a reverse complement of the nucleotide sequence represented by A; 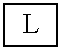
represents a “loop” encoding structure comprising a sequence of 5 to 20 non-self-complementary nucleotides; and 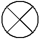
represents a terminator sequence.

The ddRNAi agent generated by the expression of the ddRNAi expression cassette represented by general structure (I) comprises a stem-loop structured precursor (shRNA) in which the ends of the double-stranded RNA are connected by a single-stranded, linker RNA. The length of the single-stranded loop portion of the shRNA may be 5 to 20 bp in length, and is preferably 5 to 9 bp in length. Accordingly, in a preferred embodiment, L in general structure (I) comprises. 5, 6, 7, 8 or 9 non-self-complementary nucleotides.

In another embodiment, the ddRNAi expression cassette comprises a nucleic acid molecule of the general structure (II): 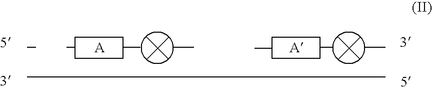
wherein:
represents a promoter sequence; 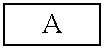
represents a ddRNAi targeting sequence comprising at least 10 nucleotides, wherein said sequence is at least about 70% identical to a portion of a Cbl gene, including a sequence known to knock out or knock down c-Cbl and/or increase phosphorylation of ACC and/or decrease activity of ACC and/or increase phosphorylation of AMPK and/or increase activity of AMPK and/or increase fatty acid oxidation and/or decrease free fatty acids in a cell; 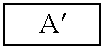
represents a sequence of 10 to 30 nucleotides wherein at least 10 contiguous nucleotides of A′ comprise a reverse complement of the nucleotide sequence represented by A; and 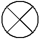
represents a terminator sequence.

In yet another embodiment, the ddRNAi expression cassette comprises a nucleic acid molecule of the general structure (III): 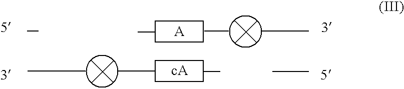
wherein:
represents a promoter sequence; 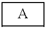
represents a ddRNAi targeting sequence comprising at least 10 nucleotides, wherein said sequence is at least about 70% identical to a portion of a Cbl gene, including a sequence known to knock out or knock down c-Cbl and/or increase phosphorylation of ACC and/or decrease activity of ACC and/or increase phosphorylation of AMPK and/or increase activity of AMPK and/or increase fatty acid oxidation and/or decrease free fatty acids in a cell; 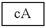
represents a nucleic acid sequence complementary to A; and 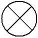
represents a terminator sequence.

In yet another preferred embodiment, the ddRNAi expression cassette comprises a nucleic acid molecule of the general structure (IV): 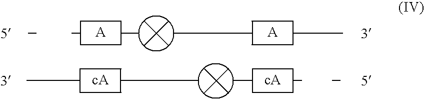
wherein:
represents a promoter sequence; 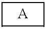
represents a ddRNAi targeting sequence comprising at least 10 nucleotides, wherein said sequence is at least about 70% identical to a portion of a Cbl gene, including a sequence known to knock out or knock down c-Cbl and/or increase phosphorylation of ACC and/or decrease activity of ACC and/or increase phosphorylation of AMPK and/or increase activity of AMPK and/or increase fatty acid oxidation and/or decrease free fatty acids in a cell; 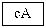
represents a nucleic acid sequence complementary to A; and 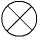
represents a terminator sequence.

Although the ddRNAi expression cassettes represented by general structures (I), (II), (III) and (IV) represent preferred embodiments of the invention, the present invention is in no way limited to these particular general structures. As would be evident to one of skill in the art, the above structures may be modified while retaining functionality. For example, the elements of the cassettes may be separated by one or more nucleotide residues. Furthermore, elements which are present on complementary strands, such as the terminator and promoter elements shown in structures (III) and (IV) may overlap or may be discreet. For example, the terminator elements shown in structure (III) may occur within the complementary strand of the promoter element or may be upstream or downstream of this region. Other modifications which would be evident to one of skill in the art and which do not materially effect the functioning of the cassette in encoding a dsRNA structure may also be made and such modified cassettes are within the scope of the present invention.

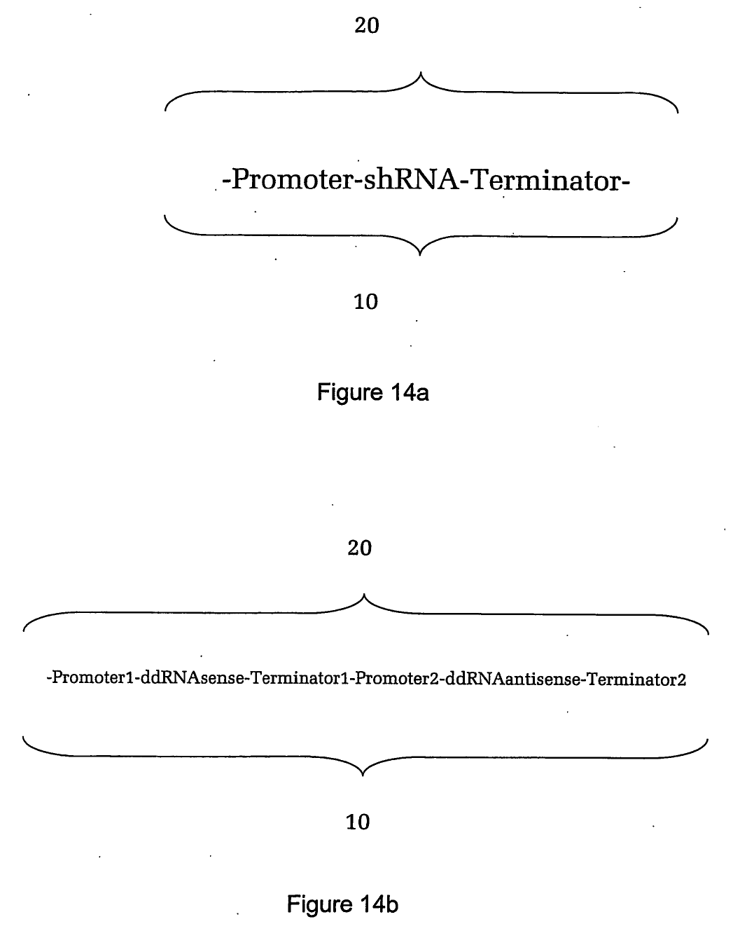

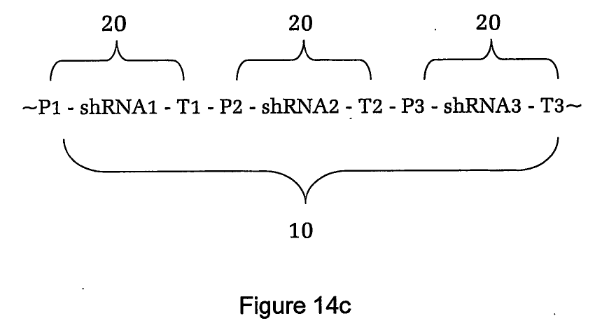

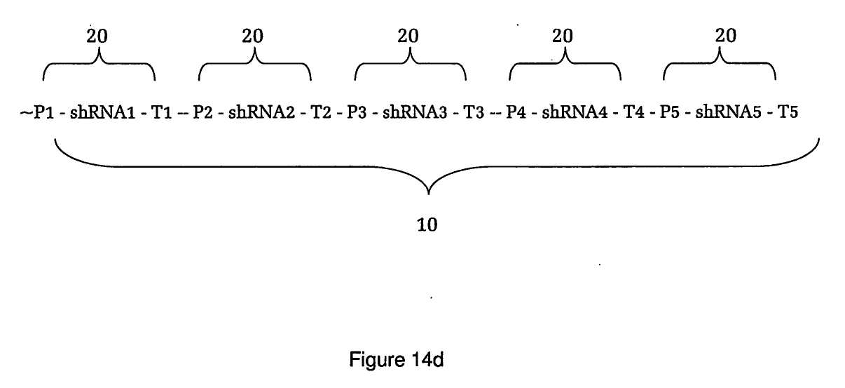

FIGS. 14a through 14d show additional examples of ddRNAi expression cassettes. FIGS. 14a and 14b are simplified schematics of single-promoter RNAi expression cassettes according to embodiments of the present invention. FIG. 14a shows an embodiment of a single RNAi expression cassette (10) comprising one promoter/RNAi/terminator component (shown at 20), where the ddRNAi agent is expressed initially as a short hairpin (shRNA). FIG. 14b shows an embodiment of a single RNAi expression cassette (10) with one promoter/RNAi/terminator component (shown at 20), where the sense and antisense components of the ddRNAi agent are expressed separately from different promoters. FIGS. 14c and 14d are simplified schematics of multiple-promoter RNAi expression cassettes according to embodiments of the present invention. FIG. 14c shows an embodiment of a multiple-promoter RNAi expression cassette (10) comprising three promoter/RNAi/terminator components (shown at 20), and FIG. 14d shows an embodiment of a multiple-promoter expression cassette (10) with five promoter/RNAi/terminator components (shown at 20). P1, P2, P3, P4 and P5 represent promoter elements. RNAi1, RNAi2, RNAi3, RNAi4 and RNAi5 represent sequences for five different ddRNAi agents. T1, T2, T3, T4, and T5 represent termination elements.

The multiple-promoter RNAi expression cassettes according to the present invention may contain two or more promoter/RNAi/terminator components where the number of promoter/RNAi/terminator components included in any multiple-promoter RNAi expression cassette is limited by, e.g., packaging size of the delivery system chosen (for example, some viruses, such as MV, have relatively strict size limitations); cell toxicity, and maximum effectiveness (i.e. when, for example, expression of four ddRNAi agents is as effective therapeutically as the expression of ten ddRNAi agents).

When employing a multiple promoter RNAi expression cassette, the two or more ddRNAi agents in the promoter/RNAi/terminator components comprising a cassette all have different sequences; that is RNAi1, RNAi2, RNAi3, RNAi4 and RNAi5 are all different from one another. However, the promoter elements in any cassette may be the same (that is, e.g., the sequence of two or more of P1, P2, P3, P4 and P5 may be the same); all the promoters within any cassette may be different from one another; or there may be a combination of promoter elements represented only once and promoter elements represented two times or more within any cassette. Similarly, the termination elements in any cassette may be the same (that is, e.g., the sequence of two or more of T1, T2, T3, T4 and T5 may be the same, such as contiguous stretches of 4 or more T residues); all the termination elements within any cassette may be different from one another; or there may be a combination of termination elements represented only once and termination elements represented two times or more within any cassette. Preferably, the promoter elements and termination elements in each promoter/RNAi/terminator component comprising any cassette are all different to decrease the likelihood of DNA recombination events between components and/or cassettes.

Further, in a preferred embodiment, the promoter element and termination element used in each promoter/RNAi/terminator component are matched to each other; that is, the promoter and terminator elements are taken from the same gene in which they occur naturally.

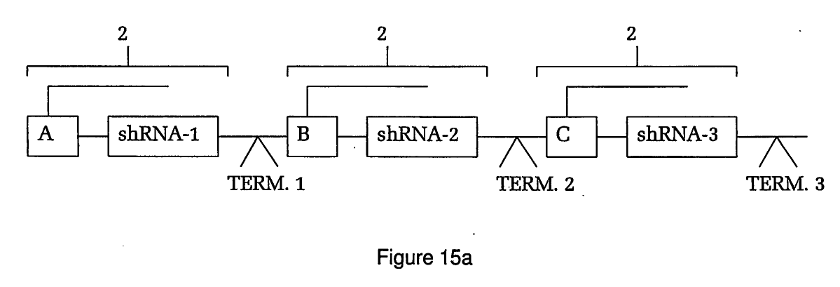

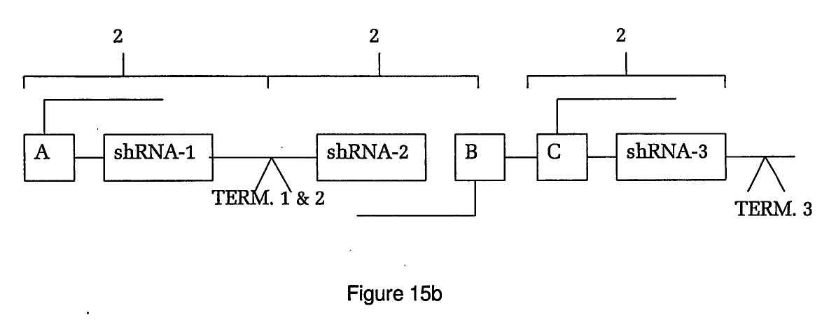

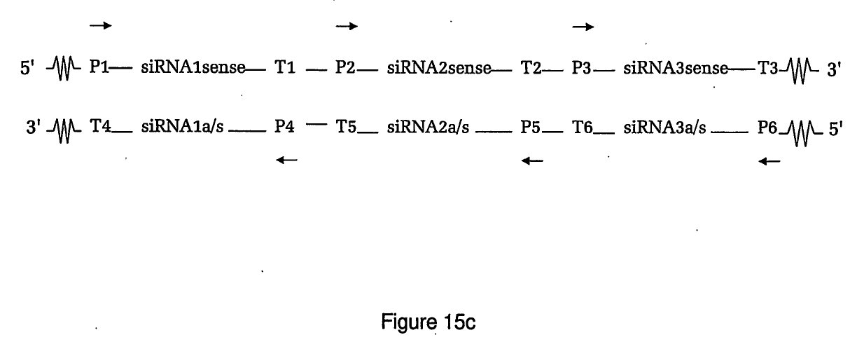

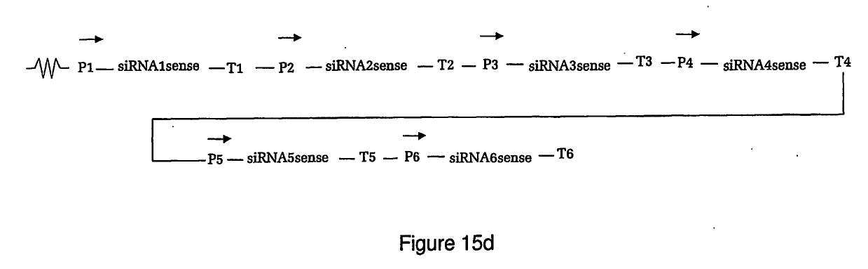

FIGS. 15a and 15b show multiple-promoter RNAi expression constructs comprising alternative embodiments of multiple-promoter RNAi expression cassettes that express short shRNAs. shRNAs are short duplexes where the sense and antisense strands are linked by a hairpin loop. Once expressed, shRNAs are processed into RNAi agents. A, B and C represent three different promoter elements, and the arrows indicate the direction of transcription. Term1, Term2, and Term3 represent three different termination sequences, and shRNA-1, shRNA-2 and shRNA-3 represent three different shRNA sequences. The multiple-promoter RNAi expression cassettes in both embodiments extend from the box marked A to the Term3. FIG. 15a shows each of the three promoter/RNAi/terminator components (20) in the same orientation within the cassette, while FIG. 15b shows the promoter/RNAi/terminator components for shRNA-1 and shRNA-3 in one orientation, and the promoter/RNAi/terminator component for sh-RNA2 in the opposite orientation (i.e., transcription takes place on both strands of the cassette). Other variations may be used.

FIGS. 15c and 15d show multiple-promoter RNAi expression constructs comprising alternative embodiments of multiple-promoter RNAi expression cassettes that express RNAi agents without a hairpin loop. In both figures, P1, P2, P3, P4, P5 and P6 represent promoter elements (with arrows indicating the direction of transcription); and T1, T2, T3, T4, T5, and T6 represent termination elements. Also in both figures, RNAi1 sense and RNAi1 antisense (a/s) are complements, RNAi2 sense and RNAi2 a/s are complements, and RNAi3 sense and RNAi3 a/s are complements. In the embodiment shown in FIG. 15c, all three RNAi sense sequences are transcribed from one strand (via P1, P2 and P3), while the three RNAi a/s sequences are transcribed from the complementary strand (via P4, P5, P6). In this particular embodiment, the termination element of RNAi1 a/s (T4) falls between promoter P1 and the RNAi1 sense sequence; while the termination element of RNAi1 sense (T1) falls between the RNAi1 a/s sequence and its promoter, P4. This motif is repeated such that if the top strand shown in FIG. 15c is designated the (+) strand and the bottom strand is designated the (−) strand, the elements encountered moving from left to right would be P1(+), T4(−), RNAi1 (sense and a/s), T1(+), P4(−), P2(+), T5(−), RNAi2 (sense and a/s), T2(+), P5(−), P3(+), T6(−), RNAi3 (sense and a/s), T3(+), and P6(−). In an alternative embodiment shown in FIG. 15d, all RNAi sense and antisense sequences are transcribed from the same strand.

One skilled in the art appreciates that any of the embodiments of the multiple-promoter RNAi expression cassettes shown in FIGS. 15a through 15d may be used for certain applications, as well as combinations or variations thereof.

The antisense RNA, ribozyme, RNAi can be introduced directly to a cell or cell-free extract capable of expressing c-Cbl as naked DNA. Alternatively, DNA encoding a nucleic acid inhibitory molecule can be introduced into a cell in operable connection with a suitable promoter and transcription terminator sequence to facilitate expression of the inhibitory nucleic acid.

Promoters of variable strength may be employed. For example, use of two or more strong promoters (such as a Pol III-type promoter) may tax the cell, by, e.g., depleting the pool of available nucleotides or other cellular components needed for transcription. In addition or alternatively, use of several strong promoters may cause a toxic level of expression of RNAi agents in the cell. Thus, in some embodiments one or more of the promoters in the multiple-promoter RNAi expression cassette may be weaker than other promoters in the cassette, or all promoters in the cassette may express RNAi agents at less than a maximum rate. Promoters also may or may not be modified using molecular techniques, or otherwise, e.g., through regulation elements, to attain weaker levels of transcription.

Promoters useful in some embodiments of the present invention may be tissue-specific or cell-specific. The term “tissue specific” as it applies to a promoter refers to a promoter that is capable of directing selective expression of a nucleotide sequence of interest to a specific type of tissue (e.g., epithelial tissue) in the relative absence of expression of the same nucleotide sequence of interest in a different type of tissue (e.g., muscle). The term “cell-specific” as applied to a promoter refers to a promoter which is capable of directing selective expression of a nucleotide sequence of interest in a specific type of cell in the relative absence of expression of the same nucleotide sequence of interest in a different type of cell within the same tissue (see, e.g., Higashibata, et al., J. Bone Miner. Res. January 19(1), 78-88, 2004; Hoggatt, et al., Circ. Res., December 91(12), 1151-59, 2002; Sohal, et al., Circ. Res. July 89(1), 20-25, 2001; and Zhang, et al., Genome Res. January 14(1), 79-89, 2004). The term “cell-specific” when applied to a promoter also means a promoter capable of promoting selective expression of a nucleotide sequence of interest in a region within a single tissue. Alternatively, promoters may be constitutive or regulatable. Additionally, promoters may be modified so as to possess different specificities.

The term “constitutive” when made in reference to a promoter means that the promoter is capable of directing transcription of an operably linked nucleic acid sequence in the absence of a specific stimulus (e.g., heat shock, chemicals, light, etc.). Typically, constitutive promoters are capable of directing expression of a coding sequence in substantially any cell and any tissue. The promoters used to transcribe the RNAi agents preferably are constitutive promoters, such as the promoters for ubiquitin, CMV, β-actin, histone H4, EF-1alfa or pgk genes controlled by RNA polymerase II, or promoter elements controlled by RNA polymerase I. In other embodiments, a Pol II promoter such as CMV, SV40, U1, β-actin or a hybrid Pol II promoter is employed. In other embodiments, promoter elements controlled by RNA polymerase III are used, such as the U6 promoters (U6-1, U6-8, U6-9, e.g.), H1 promoter, 7SL promoter, the human Y promoters (hY1, hY3, hY4 (see Maraia, et al., Nucleic Acids Res 22(15), 3045-52, 1994) and hY5 (see Maraia, et al., Nucleic Acids Res 24(18), 3552-59, 1994), the human MRP-7-2 promoter, Adenovirus VA1 promoter, human tRNA promoters, the 5s ribosomal RNA promoters, as well as functional hybrids and combinations of any of these promoters.

Alternatively in some embodiments it may be optimal to select promoters that allow for inducible expression of the RNAi agent. A number of systems for inducible expression using such promoters are known in the art, including but not limited to the tetracycline responsive system and the lac operator-repressor system (see WO 03/022052 A1; and US 2002/0162126 A1), the ecdysone regulated system, or promoters regulated by glucocorticoids, progestins, estrogen, RU-486, steroids, thyroid hormones, cyclic AMP, cytokines, the calciferol family of regulators, or the metallothionein promoter (regulated by inorganic metals).

Particularly preferred promoters for expression in mammalian cells that express Cbl include the CMV promoter, ubiquitin promoter, U6 small nuclear RNA promoter (Lee et al., Nature Biotech. 20, 500-505, 2002; Miyagishi et al., Nature Biotech 20, 497-500, 2002; Paul et al., Nature Biotech. 20, 505-508, 2002; and Yu et al., Proc. Natl Acad. Sci USA 99, 6047-6052, 2002), H1-RNA promoter (Brummelkamp et al., Science 296, 550-553, 2002), or other RNA polymerase III promoter. The pol III terminator is also preferred for such applications. Other promoters and terminators are not to be excluded.

One or more enhancers also may be present in the viral multiple-promoter RNAi expression construct to increase expression of the gene of interest. Enhancers appropriate for use in embodiments of the present invention include the Apo E HCR enhancer, the CMV enhancer that has been described recently (see, Xia et al., Nucleic Acids Res, 31-17, 2003), and other enhancers known to those skilled in the art.

The RNAi sequences encoded by the RNAi expression cassettes of the present invention result in the expression of small interfering RNAs that are short, double-stranded RNAs that are not toxic in normal mammalian cells. There is no particular limitation in the length of the ddRNAi agents of the present invention as long as they do not show cellular toxicity. RNAis can be, for example, 15 to 49 bp in length, preferably 15 to 35 bp in length, and are more preferably 19 to 29 bp in length. The double-stranded RNA portions of RNAis may be completely homologous, or may contain non-paired portions due to sequence mismatch (the corresponding nucleotides on each strand are not complementary), bulge (lack of a corresponding complementary nucleotide on one strand), and the like. Such non-paired portions can be tolerated to the extent that they do not significantly interfere with RNAi duplex formation or efficacy.

The termini of a ddRNAi agent according to the present invention may be blunt or cohesive (overhanging) as long as the ddRNAi agent effectively silences the target gene. The cohesive (overhanging) end structure is not limited only to a 3′ overhang, but a 5′ overhanging structure may be included as long as the resulting ddRNAi agent is capable of inducing the RNAi effect. In addition, the number of overhanging nucleotides may be any number as long as the resulting ddRNAi agent is capable of inducing the RNAi effect. For example, if present, the overhang may consist of 1 to 8 nucleotides, preferably it consists of 2 to 4 nucleotides.

The ddRNAi agent utilized in the present invention may have a stem-loop structured precursor (shRNA) in which the ends of the double-stranded RNA are connected by a single-stranded, linker RNA. The length of the single-stranded loop portion of the shRNA may be 5 to 20 bp in length, and is preferably 5 to 9 bp in length.

For producing shRNA from the exemplified siRNAs set forth in SEQ ID NOS: 6-241, the sense and antisense strands are positioned such that they flank an intervening loop sequence. Preferred loop sequences are selected from the group consisting of:

| | |
|---|---|
| CCC; | (SEQ ID NO: 242) |
| TTCG; | (SEQ ID NO: 243) |
| CCACC; | (SEQ ID NO: 244) |
| CTCGAG; | (SEQ ID NO: 245) |
| AAGCTT; | (SEQ ID NO: 246) |
| CCACACC; | (SEQ ID NO: 247) |
| and | |
| TTCAAGAGA. | (SEQ ID NO: 248) |

Of these loop sequences, the sequence set forth in SEQ ID NO: 248 is particularly preferred for modulating human c-Cbl expression in a cell, tissue (eg., skeletal muscle, or whole organism).

The nucleic acid sequences that are targets for the RNAi expression cassettes of the present invention include genes that are involved in obesity, anorexia, type II diabetes, atherosclerosis or heart disease in general, and more particularly, that are involved in modulating insulin action e.g., Cbl including c-Cbl, ACC and AMPK. The RNAi is preferably capable of downregulating expression of human c-Cbl in a cell. The sequences for the RNAi agent or agents are selected based upon the genetic sequence of the target gene sequence(s); and preferably are based on regions of the target gene sequences that are conserved.

Preferred RNAi molecules comprise a nucleotide sequence that is Identical to about 19-21 contiguous nucleotides of the target mRNA. Preferably, the target sequence in Cbl mRNA commences with the dinucleotide M, comprises a GC-content of about 30-70% (preferably, 30-60%, more preferably 40-60% and more preferably about 45%-55%), and does not have a high percentage identity to any nucleotide sequence other than a c-Cbl protooncogene in the genome of the animal in which it is to be introduced, e.g., as determined by standard BLAST search.

In view of the high percentage conservation between murine and human c-Cbl-encoding genes, especially in the coding regions, this should not be taken to indicate a requirement for the RNAi to be specific for human c-Cbl-encoding genes. In the cell-based and animal models described herein, it is possible and appropriate in certain circumstances for the RNAi molecules to reduce expression of both endogenous murine c-Cbl, as well as ectopically expressed human c-Cbl in the cell. Confirmation of a specific activity of any antagonist against human c-Cbl is determined by assessing the activity of an inhibitor in a cell derived from a c-Cbl−/− mouse that has been engineered to express human c-Cbl.

Preferred RNAi molecules that are selectively active against human c-Cbl expression compared to murine c-Cbl expression are derived from the sequence of the 5′-non-coding and/or 3′-non-coding region of the human c-Cbl gene. Such specific siRNAs include, e.g., SEQ ID Nos: 59-61, 119-123, 177-179 and 237-241.

Typically, inhibition of target sequences by RNAi requires a high degree of sequence homology between the target sequence and the sense strand of the RNAi molecules. In some embodiments, such homology is higher than about 70%, and may be higher than about 75%. Preferably, homology is higher than about 80%, and is higher than 85% or even 90%. More preferably, sequence homology between the target sequence and the sense strand of the RNAi is higher than about 90%, 91%, 92%, 93%, 94%, 95%, 96%, 97%, 98% or 99%.

Methods of alignment of sequences for comparison and RNAi sequence selection are well known in the art. The determination of percent identity between two or more sequences can be accomplished using a mathematical algorithm. Preferred, non-limiting examples of such mathematical algorithms are the algorithm of Myers and Miller (1988); the search-for-similarity-method of Pearson and Lipman (1988); and that of Karlin and Altschul (1993). Preferably, computer implementations of these mathematical algorithms are utilized. Such implementations include, but are not limited to: CLUSTAL in the PC/Gene program (available from Intelligenetics, Mountain View, Calif.); the ALIGN program (Version 2.0), GAP, BESTFIT, BLAST, FASTA, Megalign (using Jotun Hein, Martinez, Needleman-Wunsch algorithms), DNAStar Lasergene (see www.dnastar.com) and TFASTA in the Wisconsin Genetics Software Package, Version 8 (available from Genetics Computer Group (GCG), 575 Science Drive, Madison, Wis., USA). Alignments using these programs can be performed using the default parameters or parameters selected by the operator. The CLUSTAL program is well described by Higgins. The ALIGN program is based on the algorithm of Myers and Miller; and the BLAST programs are based on the algorithm of Karlin and Altschul. Software for performing BLAST analyses is publicly available through the National Center for Biotechnology Information of the National Institutes of Health, Bethesda, Md.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As exemplified herein, preferred siRNA against a c-Cbl encoding gene of humans comprises a 21-nucleotide sequence set forth in any one of SEQ ID Nos: 6-241. Alternatively, murine c-Cbl expression is knocked-down successfully by shRNA comprising the sequence set forth in SEQ ID NO: 268 (Example 2).

SEQ ID Nos: 6-123 each comprise (i) a 19-nucleotide sequence corresponding to a human c-Cbl mRNA target sequence, preferably adjacent and downstream of a dinucleotide AA in said mRNA target; and (ii) a 3′-extension dinucleotide TT. SEQ ID NOS: 124-241 each comprise (i) a 19-nucleotide sequence complementary to a human c-Cbl mRNA target sequence contained within SEQ ID NOS: 6-123, respectively; and (ii) a 3′-extension dinucleotide TT.

In addition to selecting the RNAi sequences based on conserved regions of a target gene, selection of the RNAi sequences may be based on other factors. Despite a number of attempts to devise selection criteria for identifying sequences that will be effective in RNAi based on features of the desired target sequence (e.g., percent GC content, position from the translation start codon, or sequence similarities based on an in silico sequence database search for homologs of the proposed RNAi, thermodynamic pairing criteria), it is presently not possible to predict with much degree of confidence which of the myriad possible candidate RNAi sequences correspond to target gene, in fact, elicit an optimal RNA silencing response. Instead, individual specific candidate RNAi polynucleotide sequences typically are generated and tested to determine whether interference with expression of a desired target can be elicited.

As stated, the ddRNAi agent coding regions of RNAi expression cassette are operatively linked to terminator elements. In one embodiment, the terminators comprise stretches of four or more thymidine residues. In embodiments where multiple promoter cassettes are used, the terminator elements used all may be different and are matched to the promoter elements from the gene from which the terminator is derived. Such terminators include the SV40 poly A, the Ad VA1 gene, the 5S ribosomal RNA gene, and the terminators for human t-RNAs. In addition, promoters and terminators may be mixed and matched, as is commonly done with RNA pol II promoters and terminators.

In addition, the RNAi expression cassettes may be configured where multiple cloning sites and/or unique restriction sites are located strategically, such that the promoter, ddRNAi agents and terminator elements are easily removed or replaced. The RNAi expression cassettes may be assembled from smaller oligonucleotide components using strategically located restriction sites and/or complementary sticky ends. The base vector for one approach according to embodiments of the present invention consists of plasmids with a multilinker in which all sites are unique (though this is not an absolute requirement). Sequentially, each promoter is inserted between its designated unique sites resulting in a base cassette with one or more promoters, all of which can have variable orientation. Sequentially, again, annealed primer pairs are inserted into the unique sites downstream of each of the individual promoters, resulting in a single-, double- or multiple-expression cassette construct. The insert can be moved into, e.g. an MV backbone using two unique enzyme sites (the same or different ones) that flank the single-, double- or multiple-expression cassette insert.

In one embodiment, the DNA encoding the inhibitory nucleic acid is operably connected to promoter and terminator regulatory sequences by cloning into a suitable vector that comprises the necessary promoter and transcriptional terminator sequences, and the recombinant vector is then introduced to the cell, tissue or organ by transient transfection of plasmid DNA, by establishing permanent cell lines or in infection with retroviral expression vectors (Barton et al., Proc. Natl Acad. Sci USA 99, 14943-14945, 2002; Devroe et al., BMC Biotech. 2, p 15, 2002).

When using a ddRNAi agent, the RNAi expression cassette is ligated into a delivery vector. The constructs into which the RNAi expression cassette is inserted and used for high efficiency transduction and expression of the ddRNAi agents in various cell types may be derived from viruses and are compatible with viral delivery; alternatively, non-viral delivery method may be used. Generation of the construct can be accomplished using any suitable genetic engineering techniques well known in the art, including without limitation, the standard techniques of PCR, oligonucleotide synthesis, restriction endonuclease digestion, ligation, transformation, plasmid purification, and DNA sequencing. If the construct is a viral construct, the construct preferably comprises, for example, sequences necessary to package the RNAi expression construct into viral particles and/or sequences that allow integration of the RNAi expression construct into the target cell genome. The viral construct also may contain genes that allow for replication and propagation of virus, though in other embodiments such genes will be supplied in trans. Additionally, the viral construct may contain genes or genetic sequences from the genome of any known organism incorporated in native form or modified. For example, a preferred viral construct may comprise sequences useful for replication of the construct in bacteria.

The construct also may contain additional genetic elements. The types of elements that may be included in the construct are not limited in any way and may be chosen by one with skill in the art. For example, additional genetic elements may include a reporter gene, such as one or more genes for a fluorescent marker protein such as GFP or RFP; an easily assayed enzyme such as beta-galactosidase, luciferase, beta-glucuronidase, chloramphenical acetyl transferase or secreted embryonic alkaline phosphatase; or proteins for which immunoassays are readily available such as hormones or cytokines. Other genetic elements that may find use in embodiments of the present invention include those coding for proteins which confer a selective growth advantage on cells such as adenosine deaminase, aminoglycodic phosphotransferase, dihydrofolate reductase, hygromycin-B-phosphotransferase, drug resistance, or those genes coding for proteins that provide a biosynthetic capability missing from an auxotroph. If a reporter gene is included along with the RNAi expression cassette, an internal ribosomal entry site (IRES) sequence can be included. Preferably, the additional genetic elements are operably linked with and controlled by an independent promoter/enhancer. In addition a suitable origin of replication for propagation of the construct in bacteria may be employed. The sequence of the origin of replication generally is separated from the ddRNAi agent and other genetic sequences that are to be expressed in the epithelial tissue. Such origins of replication are known in the art and include the pUC, ColE1, 2-micron or SV40 origins of replication.

A viral delivery system based on any appropriate virus may be used to deliver the RNAi expression constructs of the present invention. In addition, hybrid viral systems may be of use. The choice of viral delivery system will depend on various parameters, such as efficiency of delivery into epithelial tissue, transduction efficiency of the system, pathogenicity, immunological and toxicity concerns, and the like. It is clear that there is no single viral system that is suitable for all applications. When selecting a viral delivery system to use in the present invention, it is important to choose a system where RNAi expression construct-containing viral particles are preferably: 1) reproducibly and stably propagated; 2) able to be purified to high titers; and 3) able to mediate targeted delivery (delivery of the multiple-promoter RNAi expression construct to the epithelial tissue without widespread dissemination).

In general, the five most commonly used classes of viral systems used in gene therapy can be categorized into two groups according to whether their genomes integrate into host cellular chromatin (oncoretroviruses and lentiviruses) or persist in the cell nucleus predominantly as extrachromosomal episomes (adeno-associated virus, adenoviruses and herpesviruses).

In one preferred embodiment of the present invention, viruses from the Parvoviridae family are utilized. The Parvoviridae is a family of small single-stranded, non-enveloped DNA viruses with genomes approximately 5000 nucleotides long. Included among the family members is adeno-associated virus (AAV), a dependent parvovirus that by definition requires co-infection with another virus (typically an adenovirus or herpesvirus) to initiate and sustain a productive infectious cycle. In the absence of such a helper virus, AAV is still competent to infect or transducer a target cell by receptor-mediated binding and internalization, penetrating the nucleus in both non-dividing and dividing cells.

Once in the nucleus, the virus uncoats and the transgene is expressed from a number of different forms—the most persistent of which are circular monomers. MV will integrate into the genome of 1-5% of cells that are stably transduced (Nakai, et al., J. Virol. 76:11343-349 (2002)). Expression of the transgene can be exceptionally stable and in one study with MV delivery of Factor IX, a dog model continues to express therapeutic levels of the protein 4.5 years after a single direct infusion with the virus. Because progeny virus is not produced from MV infection in the absence of helper virus, the extent of transduction is restricted only to the initial cells that are infected with the virus. It is this feature that makes MV a preferred gene therapy vector for the present invention. Furthermore, unlike retrovirus, adenovirus, and herpes simplex virus, MV appears to lack pathogenicity and toxicity (Kay, et al., Nature. 424: 251 (2003) and Thomas, et al., Nature Reviews, Genetics 4:346-58 (2003)).

Typically, the genome of MV contains only two genes. The “rep” gene codes for at least four separate proteins utilized in DNA replication. The “cap” gene product is spliced differentially to generate the three proteins that comprise the capsid of the virus. When packaging the genome into nascent virus, only the Inverted Terminal Repeats (ITRs) are obligate sequences; rep and cap can be deleted from the genome and be replaced with heterologous sequences of choice. However, in order produce the proteins needed to replicate and package the MV-based heterologous construct into nascent virion, the rep and cap proteins must be provided in trans. The helper functions normally provided by co-infection with the helper virus, such as adenovirus or herpesvirus mentioned above, also can be provided in trans in the form of one or more DNA expression plasmids. Since the genome normally encodes only two genes it is not surprising that, as a delivery vehicle, MV is limited by a packaging capacity of 4.5 single stranded kilobases (kb). However, although this size restriction may limit the genes that can be delivered for replacement gene therapies, it does not adversely affect the packaging and expression of shorter sequences such as RNAi.

The utility of MV for RNAi applications was demonstrated in experiments where MV was used to deliver shRNA in vitro to inhibit p53 and Caspase 8 expression (Tomar et al., Oncogene. 22: 5712-15 (2003)). Following cloning of the appropriate sequences into a gutted MV-2 vector, infectious MV virions were generated in HEK293 cells and used to infect HeLa S3 cells. A dose-dependent decrease of endogenous Caspase 8 and p53 levels was demonstrated. Boden et al. also used MV to deliver shRNA in vitro to inhibit HIV replication in tissue culture systems (Boden, et al., J. Virol. 77(21): 115231-35 (2003)) as assessed by p24 production in the spent media.

However, technical hurdles must be addressed when using MV as a vehicle for RNAi expression constructs. For example, various percentages of the human population may possess neutralizing antibodies against certain MV serotypes. However, since there are several MV serotypes, some of which the percentage of individuals harboring neutralizing antibodies is vastly reduced, other serotypes can be used or pseudo-typing may be employed. There are at least eight different serotypes that have been characterized, with dozens of others, which have been isolated but have been less well described. Another limitation is that as a result of a possible immune response to MV, MV-based therapy may only be administered once; however, use of alternate, non-human derived serotypes may allow for repeat administrations. Administration route, serotype, and composition of the delivered genome all influence tissue specificity.

Another limitation in using unmodified MV systems with the RNAi expression constructs is that transduction can be inefficient. Stable transduction in vivo may be limited to 5-10% of cells. However, different methods are known in the art to boost stable transduction levels. One approach is utilizing pseudotyping, where MV-2 genomes are packaged using cap proteins derived from other serotypes. For example, by substituting the MV-5 cap gene for its MV-2 counterpart, Mingozzi et al. increased stable transduction to approximately 15% of hepatocytes (Mingozzi, et al., J. Virol. 76(20): 10497-502 (2002)). Thomas et al., transduced over 30% of mouse hepatocytes in vivo using the AAV8 capsid gene (Thomas, et al., J. Virol. in press). Grimm et al. (Blood. 2003-02-0495) exhaustively pseudotyped MV-2 with MV-1, MV-3B, MV-4, MV-5, and MV-6 for tissue culture studies. The highest levels of transgene expression were induced by virion which had been pseudotyped with MV-6; producing nearly 2000% higher transgene expression than MV-2. Thus, the present invention contemplates use of a pseudotyped MV virus to achieve high transduction levels, with a corresponding increase in the expression of the RNAi multiple-promoter expression constructs.

Another viral delivery system useful with the RNAi expression constructs of the present invention is a system based on viruses from the family Retroviridae. Retroviruses comprise single-stranded RNA animal viruses that are characterized by two unique features. First, the genome of a retrovirus is diploid, consisting of two copies of the RNA. Second, this RNA is transcribed by the virion-associated enzyme reverse transcriptase into double-stranded DNA. This double-stranded DNA or provirus can then integrate into the host genome and be passed from parent cell to progeny cells as a stably-integrated component of the host genome.

In some embodiments, lentiviruses are the preferred members of the retrovirus family for use in the present invention. Lentivirus vectors are often pseudotyped with vesicular stomatitis virus glycoprotein (VSV-G), and have been derived from the human immunodeficiency virus (HIV), the etiologic agent of the human acquired immunodeficiency syndrome (AIDS); visan-maedi, which causes encephalitis (visna) or pneumonia in sheep; equine infectious anemia virus (EIAV), which causes autoimmune hemolytic anemia and encephalopathy in horses; feline immunodeficiency virus (FIV), which causes immune deficiency in cats; bovine immunodeficiency virus (BIV) which causes lymphadenopathy and lymphocytosis in cattle; and simian immunodeficiency virus (SIV), which causes immune deficiency and encephalopathy in non-human primates. Vectors that are based on HIV generally retain <5% of the parental genome, and <25% of the genome is incorporated into packaging constructs, which minimizes the possibility of the generation of reverting replication-competent HIV. Biosafety has been further increased by the development of self-inactivating vectors that contain deletions of the regulatory elements in the downstream long-terminal-repeat sequence, eliminating transcription of the packaging signal that is required for vector mobilization.

Reverse transcription of the retroviral RNA genome occurs in the cytoplasm. Unlike C-type retroviruses, the lentiviral cDNA complexed with other viral factors—known as the pre-initiation complex—is able to translocate across the nuclear membrane and transduce non-dividing cells. A structural feature of the viral cDNA—a DNA flap—seems to contribute to efficient nuclear import. This flap is dependent on the integrity of a central polypurine tract (cPPT) that is located in the viral polymerase gene, so most lentiviral-derived vectors retain this sequence. Lentiviruses have broad tropism, low inflammatory potential, and result in an integrated vector. The main limitations are that integration might induce oncogenesis in some applications. The main advantage to the use of lentiviral vectors is that gene transfer is persistent in most tissues or cell types.

A lentiviral-based construct used to express the ddRNAi agents preferably comprises sequences from the 5′ and 3′ LTRs of a lentivirus. More preferably the viral construct comprises an inactivated or self-inactivating 3′ LTR from a lentivirus. The 3′ LTR may be made self-inactivating by any method known in the art. In a preferred embodiment, the U3 element of the 3′ LTR contains a deletion of its enhancer sequence, preferably the TATA box, Sp1 and NF-kappa B sites. As a result of the self-inactivating 3′ LTR, the provirus that is integrated into the host cell genome will comprise an inactivated 5′ LTR. The LTR sequences may be LTR sequences from any lentivirus from any species. The lentiviral-based construct also may incorporate sequences for MMLV or MSCV, RSV or mammalian genes. In addition, the U3 sequence from the lentiviral 5′ LTR may be replaced with a promoter sequence in the viral construct. This may increase the titer of virus recovered from the packaging cell line. An enhancer sequence may also be included.

Other viral or non-viral systems known to those skilled in the art may be used to deliver the RNAi expression cassettes of the present invention to epithelial tissue, including but not limited to gene-deleted adenovirus-transposon vectors that stably maintain virus-encoded transgenes in vivo through integration into host cells (see Yant, et al., Nature Biotech. 20:999-1004 (2002)); systems derived from Sindbis virus or Semliki forest virus (see Perri, et al, J. Virol. 74(20):9802-07 (2002)); systems derived from Newcastle disease virus or Sendai virus; or mini-circle DNA vectors devoid of bacterial DNA sequences (see Chen, et al., Molecular Therapy. 8(3):495-500 (2003)).

In addition, hybrid viral systems may be used to combine useful properties of two or more viral systems. For example, the site-specific integration machinery of wild-type AAV may be coupled with the efficient internalization and nuclear targeting properties of adenovirus. AAV in the presence of adenovirus or herpesvirus undergoes a productive replication cycle; however, in the absence of helper functions, the AAV genome integrates into a specific site on chromosome 19. Integration of the AAV genome requires expression of the AAV rep protein. As conventional rAAV vectors are deleted for all viral genes including rep, they are not able to specifically integrate into chromosome 19. However, this feature may be exploited in an appropriate hybrid system. In addition, non-viral genetic elements may be used to achieve desired properties in a viral delivery system, such as genetic elements that allow for site-specific recombination.

In high throughput primary assays at least, it is preferred to use an in vitro cell-free system or cell-based system in which Cbl activity is assayed. Several vectors are known for this purpose, including, for example, the pSilencer series of vectors (pSilencer 2.0, pSilencer 2.1, pSilencer 3.0, pSilencer 3.1, pSilencer 1.0-U6) provided by Ambion.

Preferred retroviral vectors, suitable for transiently transfecting into isolated cells e.g., by calcium phosphate precipitation (Ketteler et al., Gene Ther. 9, 477-487, 2002) in high throughput screens, or for the production of transducing supernatants (Ketteler et al., Gene Ther. 9, 477-487, 2002) for lower-throughput screening or validation of primary screen results, include pBABE (Morgenstern et al., Nuc. Acids Res. 18, 3587-3596, 1990) and JZenNeo.

The pBabe retroviral vector constructs transmit inserted genes at high titres and express them from the Mo MuLV Long Terminal Repeat (LTR). The pBabe vectors comprise one of four different dominantly-acting selectable markers, allowing the growth of infected mammalian cells in the presence of G418, hygromycin B, bleomycin/phleomycin or puromycin. The high titre ecotropic helper free packaging cell line, omega E, reduces the risk of generation of wild type Mo MuLV via homologous recombination events. Together, the pBabe vectors and omega E cell line provide high frequency gene transfer, and/or concomitant expression of c-Cbl with one or more other genes (e.g., APS and/or Insulin receptor β-subunit) in a single cell, with minimal risk of helper virus contamination.

For lower throughput primary screening or validation assays, the adenoviral vectors pAdTrack and pAdTrack-CMV (He et al., Proc. Natl Acad. Sci USA 95, 2509-2514, 1998; pAdTrack-HP (Zhao et al., Gene 316, 137-141, 2003), an Ad5CMV-based vector e.g., Ad5CMV-GFP (Suoka et al., Am. J: Respir. Cell Mol. Biol. 23, 297-303, 2000), and pSilencer adeno 1.0-CMV (Ambion) are preferred for delivery and expression in specific organs or tissues, in particular muscle tissue of a mouse model.

The pAdTrack and pAdTrack-CMV vectors are particularly preferred for applications which require standardization for transfection or transduction efficiency eg., injection of adenovirus into hindlimb muscles of transgenic mouse models. The pAdTrack vector is used for production of GFP-trackable viruses containing transgenes under the control of a chosen promoter. It contains the gene encoding enhanced GFP, a polylinker for insertion of exogenous transgenes surrounded by adenoviral sequences (“arms”) that allow homologous recombination with pAdEasy-1. The left arm contains Ad5 nucleotides 34,931-35,935, which mediate homologous recombination with pAdEasy vectors in E. coli, plus inverted terminal repeat (ITR) and packaging signal sequences (nucleotides 1-480 of Ad5) required for viral production in mammalian cells. The right arm contains Ad5 nucleotides 3,534-5,790, which mediate homologous recombination with pAdEasy vectors. Artificially created PacI sites surround both arms. The AdTrack plasmid also contains a kanamycin resistance gene from pZero 2.1 (Invitrogen) and the origin of replication from pBR322 (Life Technologies). The relatively low copy number of plasmids generated with this origin is essential for the stability of large constructs in E. coli. The pAdTrack-CMV vector is identical to pAdTrack except for the addition of a cytomegalovirus (CMV) promoter and polyadenylation site (both from pEGFP-C1, Clontech). A polylinker is present between the CMV promoter and polyadenylation site.

As will be known to the skilled artisan, such adenoviral vectors are also suitable for transfection of cell lines.

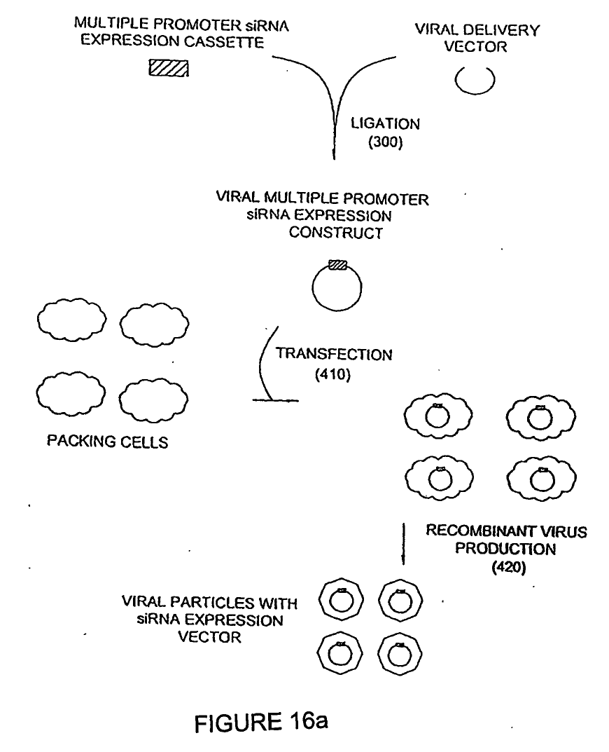

The RNAi expression construct is generally packaged into viral particles. Any method known in the art may be used to produce infectious viral particles whose genome comprises a copy of the viral RNAi expression construct. FIGS. 16a and 16b show alternative methods for packaging the RNAi expression constructs of the present invention into viral particles for delivery. The method in FIG. 16a utilizes packaging cells that stably express in trans the viral proteins that are required for the incorporation of the viral RNAi expression construct into viral particles, as well as other sequences necessary or preferred for a particular viral delivery system (for example, sequences needed for replication, structural proteins and viral assembly) and either viral-derived or artificial ligands for tissue entry. In FIG. 16a, a RNAi expression cassette is ligated to a viral delivery vector, and the resulting viral RNAi expression construct is used to transfect packaging cells. The packaging cells then replicate viral sequences, express viral proteins and package the viral RNAi expression constructs into infectious viral particles. The packaging cell line may be any cell line that is capable of expressing viral proteins, including but not limited to 293, HeLa, A549, PerC6, D17, MDCK, BHK, bing cherry, phoenix, Cf2Th, or any other line known to or developed by those skilled in the art. One packaging cell line is described, for example, in U.S. Pat. No. 6,218,181.

Alternatively, a cell line that does not stably express necessary viral proteins may be co-transfected with two or more constructs to achieve efficient production of functional particles. One of the constructs comprises the viral RNAi expression construct, and the other plasmid(s) comprises nucleic acids encoding the proteins necessary to allow the cells to produce functional virus (replication and packaging construct) as well as other helper functions. The method shown in FIG. 16b utilizes cells for packaging that do not stably express viral replication and packaging genes. In this case, the RNAi expression construct is ligated to the viral delivery vector and then co-transfected with one or more vectors that express the viral sequences necessary for replication and production of infectious viral particles. The cells replicate viral sequences, express viral proteins and package the viral RNAi expression constructs into infectious viral particles.

The packaging cell line or replication and packaging construct may not express envelope gene products. In these embodiments, the gene encoding the envelope gene can be provided on a separate construct that is co-transfected with the viral RNAi expression construct. As the envelope protein is responsible, in part, for the host range of the viral particles, the viruses may be pseudotyped. As described supra, a “pseudotyped” virus is a viral particle having an envelope protein that is from a virus other than the virus from which the genome is derived. One with skill in the art can choose an appropriate pseudotype for the viral delivery system used and cell to be targeted. In addition to conferring a specific host range, a chosen pseudotype may permit the virus to be concentrated to a very high titer. Viruses alternatively can be pseudotyped with ecotropic envelope proteins that limit infection to a specific species (e.g., ecotropic envelopes allow infection of, e.g., murine cells only, where amphotropic envelopes allow infection of, e.g., both human and murine cells.) In addition, genetically-modified ligands can be used for cell-specific targeting, such as the asialoglycoprotein for hepatocytes, or transferrin for receptor-mediated binding.

After production in a packaging cell line, the viral particles containing the RNAi expression cassettes are purified and quantified (titered). Purification strategies include density gradient centrifugation, or, preferably, column chromatographic methods.

Multiple-promoter RNAi expression cassettes used in certain embodiments of the present invention are particularly useful in treating obesity, type II diabetes, atherosclerosis or heart disease, because RNAi agents against multiple genes involved in obesity, type II diabetes, atherosclerosis or heart disease, can be targeted simultaneously. For example, one or more genes that regulate blood clotting and/or epithelial hyperproliferation can be repressed at the same time.

A variety of techniques are available and well known for delivery of nucleic acids into cells, for example liposome- or micelle-mediated transfection or transformation, transformation of cells with attenuated virus particles or bacterial cells, cell mating, transformation or transfection procedures known to those skilled in the art or microinjection. The RNAi agents, whether siRNAs or ddRNAs, are formulated with an appropriate carrier, which may then be associated with a delivery vehicle such as a matrix such as a compress, ultimately forming a therapeutic device. The delivery vehicle may be the carrier itself.

Dominant Negative Mutants

The term “dominant-negative mutant” refers to a Cbl polypeptide that has been mutated from its natural state and that interacts with a protein that Cbl normally interacts with thereby preventing endogenous native Cbl from forming the interaction. Preferred dominant negative mutants will lack that portion of Cbl that interacts with a protein selected from the group consisting of CAP, CrkII and C3G.

Preferred dominant-negative mutants comprise variants of the native Cbl protein, such as, for example, substitution or deletion mutants, that act as dominant-negative mutants of Cbl function. For example, a dominant-negative mutant may comprise one or more amino acid substitutions within the RING domain of c-Cbl. When expressed in a cell, such a dominant-negative mutant protein competes with native endogous c-Cbl for the APS protein and/or insulin receptor β-subunit, however has reduced or no E3 ubiquitin ligase activity with respect to the receptor. In an alternative embodiment, the dominant-negative mutant comprises 1 or 2 or 3 substitutions of tyrosine residues that would normally be phosphorylated in the cell. Means for producing mutated nucleic acid are well known to those skilled in the art and may be achieved readily e.g., using the Quick Change Mutagenesis kit supplied by Stratagene, La Jolla, Calif. USA according to the manufacturer's instructions.

Dominant negative mutant proteins are produced by expression of nucleic acid encoding the mutant protein, essentially as described herein above for expression of peptides in cells. The amino acid sequences of exemplary dominant-negative mutants of c-Cbl are set forth in SEQ ID NO: 250 (c-Cbl G306E), SEQ ID NO: 252 (c-Cbl C381A), SEQ ID NO: 254 (c-Cbl Y700F), SEQ ID NO: 256 (c-Cbl Y731F), SEQ ID NO: 258 (c-Cbl Y774F), SEQ ID NO: 260 (c-Cbl Y700F/Y731FY774F) and SEQ ID NO: 262 (c-Cbl 480 i.e, truncated at residue 480 of native human c-Cbl).

Antibodies

The “antibodies” contemplated herein are immunoreactive with Cbl polypeptides or functional fragments thereof. Antibodies that consist essentially of pooled monoclonal antibodies with different epitope specificities, as well as distinct monoclonal antibody preparations are contemplated. Monoclonal antibodies are produced from fragments of the Cbl protein that comprise one or more B cell epitopes by methods well known to those skilled in the art (Kohler et al, Nature 256:495, 1975). The term “antibody” as used herein includes intact molecules as well as fragments thereof, such as Fab and F(ab′)₂, Fv and single chain antibody fragments capable of binding an epitopic determinant of Cbl.

An “Fab fragment” consists of a monovalent antigen-binding fragment of an antibody molecule, and can be produced by digestion of a whole antibody molecule with the enzyme papain, to yield a fragment consisting of an intact light chain and a portion of a heavy chain.

An “Fab′ fragment” of an antibody molecule can be obtained by treating a whole antibody molecule with pepsin, followed by reduction, to yield a molecule consisting of an intact light chain and a portion of a heavy chain. Two Fab′ fragments are obtained per antibody molecule treated in this manner.

An “F(ab)₂ fragment” of an antibody consists of a dimer of two Fab′ fragments held together by two disulfide bonds, and is obtained by treating a whole antibody molecule with the enzyme pepsin, without subsequent reduction. A (Fab′).sub.2 fragment.

An “Fv fragment” is a genetically engineered fragment containing the variable region of a light chain and the variable region of a heavy chain expressed as two chains.

A “single chain antibody” (SCA) is a genetically engineered single chain molecule containing the variable region of a light chain and the variable region of a heavy chain, linked by a suitable, flexible polypeptide linker.

Antibodies or antibody fragments are generated using the entire Cbl polypeptide or an immunogenic fragment thereof (alone or linked to a suitable carrier or hapten) to immunize a subject (e.g., a mammal including, but not limited to a rabbit, goat, mouse or other mammal). For example, the methods described in U.S. Pat. Nos. 5,422,110; 5,837,268; 5,708,155; 5,723,129; and 5,849,531, can be used and are incorporated herein by reference. In a preferred embodiment, the mammal being immunized does not contain endogenous Cbl (e.g., a Cbl-deficient genetically modified animal). The immunogenic preparation can further include an adjuvant, such as Freund's complete or incomplete adjuvant, or similar immunostimulatory agent. Immunization of a suitable subject with an immunogenic proteolytic or synthetic Cbl peptide preparation induces a polyclonal anti-Cbl antibody response. The anti-Cbl antibody titer in the immunized subject is generally monitored over time by standard techniques, such as with an enzyme linked immunosorbent assay (ELISA) using immobilized Cbl. Subsequently, the sera from the immunized subjects can be tested for Cbl inhibitory activity.

Alternatively, it is also possible to immunize the subject with nucleic acid expressing Cbl using DNA immunization technology, such as that disclosed in U.S. Pat. No. 5,795,872 to Ricigliano et al., or alternatively, in U.S. Pat. No. 5,643,578 to Robinson et al.

The antibody molecules directed against Cbl can be isolated from the mammal (e.g., from the blood) and further purified by well known techniques, such as protein A chromatography to obtain the IgG fraction. At an appropriate time after immunization, e.g., when the anti-Cbl antibody titers are highest, antibody-producing cells can be obtained from the subject and used to prepare e.g., monoclonal antibodies by standard techniques, such as the hybridoma technique originally described in the following disclosures: Kohler and Milstein Nature 256:495-497, 1975; Brown et al. J. Immunol. 127:53946, 1981; Brown et al. J. Biol Chem. 255: 4980-4983, 1980; Yeh et al. Proc. Natl. Acad. Sci. USA 76:2927-2931, 1976; Yeh et al. Int. J. Cancer 29: 269-275, 1982; Kozbor et al. Immunol Today 4:72, 1983; Cole et al., Monoclonal Antibodies and Cancer Therapy, Alan R. Liss, Inc., pp. 77-96, 1985. The technology for producing monoclonal antibody hybridomas is well known in the art. Briefly, an immortal cell line (typically a myeloma) is fused to lymphocytes (typically splenocytes) from a mammal immunized with a Cbl peptide immunogen as described above, and the culture supernatants of the resulting hybridoma cells are screened to identify a hybridoma producing a monoclonal antibody that binds Cbl.

Any of the known protocols used for fusing lymphocytes and immortalized cell lines can be applied for the purpose of generating an anti-Cbl monoclonal antibody (see, e.g., G. Galfre et al., Nature 266: 550-552, 1970). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O—Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind Cbl, e.g., using a standard ELISA assay. The antibodies can then be tested for Cbl inhibitory activity.

Formulations

In one embodiment, the identified compound is formulated for administration to a non-human animal or a human. The formulations can be suitable for administration by injection by a subcutaneous, intravenous, intranasal, or intraperitoneal route. Alternatively, they can be suitable for oral administration in the form of feed additives, tablets, troches, etc.

The compounds are conveniently formulated in a suitable excipient or diluent, such as, for example, an aqueous solvent, non-aqueous solvent, non-toxic excipient, such as a salt, preservative, buffer and the like. Examples of non-aqueous solvents are propylene glycol, polyethylene glycol, vegetable oil and injectable organic esters such as ethyloleate. Aqueous solvents include water, alcoholic/aqueous solutions, saline solutions, parenteral vehicles such as sodium chloride, Ringer's dextrose, etc. Preservatives include antimicrobial, anti-oxidants, chelating agents and inert gases. The pH and exact concentration of the various components the formulation suitable for administration to the animal are adjusted according to routine skills in the art. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. For administration by inhalation, the compounds are delivered in the form of an aerosol spray from pressured container or dispenser which contains a suitable propellant, e.g., a gas such as carbon dioxide, or a nebulizer. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

Optionally, the formulation will also include a carrier, such as, for example, bovine serum albumin (BSA), keyhole limpet hemocyanin (KLH), ovalbumin, mouse serum albumin, rabbit serum albumin and the like. Means for conjugating peptides to carrier proteins are also well known in the art and include glutaraldehyde, m-maleimidobencoyl-N-hydroxysuccinimide ester, carbodiimide and bis-biazotized benzidine.

Protein-Based Determination of ACC and/or AMPK Activities and Phosphorylation

As phosphorylation of ACC and AMPK regulates their activities, the activity of these enzymes may be determined by detecting their phosphorylation. Methods of detecting a phosphorylated protein or determining the level of a phosphorylated protein are known in the art. As exemplified herein, a phosphorylated ACC and/or AMPK specific antibodies are used to detect the amount of phosphorylated ACC and/or AMPK, respectively, in a sample. Polyclonal anti-phosphorylated ACC and AMPK antibodies are publicly available e.g., from Cell Signalling Technologies Inc. (Beverly, Mass., USA) or from Upstate Biochemicals (Lake Placid, N.Y., USA). Preferably, the amount of phosphorylated ACC/AMPK is determined using an ELISA assay as described herein, however, the amount of phosphorylated ACC/AMPK may be determined using any method known in the art, and/or described, for example, in Scopes (In: Protein Purification: Principles and Practice, Third Edition, Springer Verlag, 1994).

As will be apparent to the skilled artisan any method that detects the amount of a protein in a sample using an antibody or ligand may be adapted to detect the amount of a phosphorylated protein in a sample, eg. Western blotting, immunohistochemistry, immunofluorescence, RIA, EIA, a method utilizing a biosensor or a method using a biochip. Clearly such methods are encompassed by the instant invention.

Methods of determining ACC and aMPK enzyme activities are also known in the art or described herein. For example, ACC2 produces malonyl-CoA that inhibits carnitine palmitoyl-CoA transferase 1 (CPT1) activity, and Cakes et al, J. Exp. Zool. 281:6-11 describe an assay for the measurement of CPT1 activity that may be used to determine ACC activity.

Alternatively, AMPK and ACC regulate the expression of several proteins involved in glucose metabolism. Accordingly, an assay that determines the expression of such a protein may be used to determine ACC and/or AMPK activity. For example, an immunoassay is used to detect the amount of UCP3 protein in a sample, more preferably an ELISA assay as described herein. Antibodies particularly useful for detecting expression of ACC regulated genes are available commercially, for example, an anti-cytochrome C antibody is available from Research Diagnostics, Flanders, N.J., USA. An anti-UCP antibody is available from USBiologicals, Swampscott, USA. An anti-insulin receptor antibody is available from Chemicon, Temecula, Calif., USA. An anti-GLUT4 antibody is available from Novus Biologicals, Littleton, Colo., USA.

Nucleic Acid-Based Detection Methods for Determining ACC Expression, AMPK Activity and ACC Activity

To determine ACC expression, the level of ACC-encoding mRNA can be determined directly. Alternatively, because phosphorylation of AMPK increases activity of the enzyme, leading to phosphorylation of ACC associated with increased expression of UCP3, cytochrome C, insulin receptor and GLUT4, assays that determines the level of expression of those downstream genes can be used to determine ACC activity and/or AMPK activity.

Methods of determining the expression of a gene product (ie. mRNA or protein) are known in the art, such as, for example, in situ hybridization, quantitative PCR and microarray technology. All such assay formats are encompassed by the present invention.

In a preferred embodiment, the level of nucleic acid is determined by hybridizing a nucleic acid probe to a nucleic acid in a sample under at least low stringency hybridization conditions and detecting the hybridization using a detection means.

For nucleic acid hybridization-based approaches, shorter probes are hybridized at lower stringency hybridization (ie. reduced temperature and/or higher salt concentration and/or higher detergent concentration) than longer nucleic acid probes. Generally, hybridization is carried out well below the calculated melting temperature (Tm) of a DNA duplex comprising the probe. For example, the oligonucleotide probes exemplified herein have calculated Tm values in the range of about 55° C. to about 60° C., suggesting that hybridization involving such probes should be carried out at a temperature in the range of ambient temperature to about 45° C., and more preferably between about 40° C. to about 45° C. (ie. low stringency to moderate stringency conditions). This contrasts with standard hybridization temperatures of about 65° C. for nucleic acid probes of about 100 nucleotides or longer (ie. moderate to high stringency hybridization conditions).

For the purposes of defining the level of stringency to be used in these diagnostic assays, a low stringency is defined herein as being a hybridization and/or a wash carried out in 6×SSC buffer, 0.1% (w/v) SDS at 28° C., or equivalent conditions. A moderate stringency is defined herein as being a hybridization and/or washing carried out in 2×SSC buffer, 0.1% (w/v) SDS at a temperature in the range 45° C. to 65° C., or equivalent conditions. A high stringency is defined herein as being a hybridization and/or wash carried out in 0.1×SSC buffer, 0.1% (w/v) SDS, or lower salt concentration, and at a temperature of at least 65° C., or equivalent conditions. Reference herein to a particular level of stringency encompasses equivalent conditions using wash/hybridization solutions other than SSC known to those skilled in the art.

Generally, the stringency is increased by reducing the concentration of SSC buffer, and/or increasing the concentration of SDS and/or increasing the temperature of the hybridization and/or wash. Those skilled in the art will be aware that the conditions for hybridization and/or wash may vary depending upon the nature of the hybridization matrix used to support the sample DNA, or the type of hybridization probe used.

In one embodiment, the sample or the probe is immobilized on a solid matrix or surface (e.g., nitrocellulose or a microtitre plate). For high throughput screening, the sample or probe will generally comprise an array of nucleic acids on glass or other solid matrix, such as, for example, as described in WO 96/17958. Techniques for producing high density arrays are described, for example, by Fodor et al., Science 767-773, 1991, and in U.S. Pat. No. 5,143,854. Typical protocols for other assay formats can be found, for example in Current Protocols In Molecular Biology, Unit 2 (Northern Blotting), Unit 4 (Southern Blotting), and Unit 18 (PCR Analysis), Frederick M. Ausubul et al. (ed)., 1995.

The detection means according to this aspect of the invention may be any nucleic acid-based detection means such as, for example, nucleic acid hybridization or amplification reaction (eg. PCR), a nucleic acid sequence-based amplification (NASBA) system, inverse polymerase chain reaction (iPCR), or in situ polymerase chain reaction.

The probe can be labelled with a reporter molecule capable of producing an identifiable signal (e.g., a radioisotope such as ³²P or ³⁵S, or a fluorescent or biotinylated molecule, or a coloured dye e.g. TAMRA, FAM, ROC, etc). According to this embodiment, those skilled in the art will be aware that the detection of said reporter molecule provides for identification of the probe and that, following the hybridization reaction, the detection of the corresponding nucleotide sequences in the sample is facilitated. Additional probes can be used to confirm the assay results obtained using a single probe.

Wherein the detection means is an amplification reaction such as, for example, a polymerase chain reaction or a nucleic acid sequence-based amplification (NASBA) system or a variant thereof, one or more nucleic acid probes molecules of at least about 20 contiguous nucleotides in length is hybridized to genomic DNA and nucleic acid copies of the template are enzymatically-amplified.

Those skilled in the art will be aware that there must be a sufficiently high percentage of nucleotide sequence identity between the probes and the nucleotide sequence of the sample template molecule for hybridization to occur. As stated previously, the stringency conditions can be selected to promote hybridization.

In one format, PCR provides for the hybridization of non-complementary probes to different strands of a double-stranded nucleic acid template molecule (ie. a DNA/DNA template), such that the hybridized probes are positioned to facilitate the 5′- to 3′ synthesis of nucleic acid in the intervening region, under the control of a thermostable DNA polymerase enzyme. In accordance with this embodiment, one sense probe and one antisense probe as described herein is used.

Variations of the embodiments described herein are described in detail by McPherson et al., PCR: A Practical Approach. (series eds, D. Rickwood and B. D. Hames), IRL Press Limited, Oxford. pp 1-253, 1991.

Determination of Fatty Acid Synthesis and Oxidation

Methods of determining fatty acid synthesis are known in the art and include, for example, incubating a cell lysate, eg. derived from a genetically modified organism, with carbon ([14C]malonyl-CoA) and either NADH or NAPDH, with or without putative modulatory compound. Incorporation of malonyl-CoA into fatty acids was measured by liquid scintillation counting

Methods of determining Fatty acid oxidation are known in the art and include, for example, measuring β-oxidation of fatty acids using [³H]-palmitate as a substrate in a muscle or a hepatocyte isolated from a subject (eg. a genetically modified organism) essentially as described by Alam and Saggerson, Biochem. J., 334: 233-241, 1998 or Moon and Rhead, J. Clin. Invest., 79: 59-64.

Determination of E3 Ubiquitin Ligase Activity

In an alternative embodiment, the screening assays supra are performed in vitro using a format that determines the E3 ubiquitin ligase activity of Cbl. Such assays are based upon the knowledge that the APS adapter protein couples the precursor insulin receptor p-subunit to the phosphorylation of c-Cbl and facilitates ligand-stimulated ubiquitination of the insulin receptor (IR-β) (Ahmed et al., FEBS Letts 475, 31-34, 2000). Accordingly, to determine ubiquitination of the insulin receptor by c-Cbl, cells that either express APS, c-Cbl and IR-β are required. CHO.T cells are particularly preferred for this purpose, because they are known to over express the IR-β precursor protein.

In one embodiment, a test sample is contacted with a test compound under suitable test conditions, wherein the test sample comprises APS protein, c-Cbl protein, insulin receptor (IR-β) protein, an EI enzyme, a ubiquitin conjugating enzyme, ubiquitin and adenosine 5-triphosphate (ATP). The ubiquitination of IR-β is determined and the level of ubiquitination of IR-β in the test sample with the ubiquitination of IR-β incubated under the same test conditions in the absence of test compound is determined. The level of ubiquitination of IR-β in the test sample is indicative of the ability of the test compound to modulate ubiquitin ligase activity of c-Cbl. An increase in the level of ubiquitination is an indication that ubiquitin ligase activity of c-Cbl has been enhanced, whereas a decrease in the level of ubiquitination is an indication that ubiquitin ligase activity of c-Cbl has been inhibited.

E1 enzyme is well known to one of skill in the art (e.g., Hershko et al., Ann. Rev. Biochem. 61:761-807, 1992, and Monia et al., Biotechnol. 8: 209-215, 1990, herein incorporated by reference). E1 enzyme initiates the ubiquitination process by activating ubiquitin. Any of the E1 enzymes known in the art are suitable for use.

Ubiquitin conjugating enzymes transfer ubiquitin to lysine residues of suitable substrates. They also undergo auto-ubiquitination. Suitable ubiquitin conjugating enzymes that can be employed in the invention method include Cdc34, UbcH1, UbCH2, UbCH3, UbCH4, UbCH5, UbCH6, UbCH7, UbcH10, L-UBC, and the like (see Kaiser, et al, FEBS Letts 350:1-4, 1994; Kaiser, et al, FEBS Letts 377:193-196, 1995; Nuber, et al, J Biol Chem 271:2795-2800, 1996; Jensen, et al, J Biol Chem 270:30408-30414, 1995; Robinson, et al, Mamm Genome 6:725-731, 1995; and Plon et al., Proc. Natl. Acad. Sci. USA 90:10484-10488, all of which are herein incorporated by reference). “Cdc34” refers to a ubiquitin-conjugating enzyme isolated from yeast.

In one embodiment, the ubiquitin is a derivatized ubiquitin conjugated to a label that is readily identified. For example, the derivatized ubiquitin can be [¹²⁵I]-ubiquitin, a fluorescent ubiquitin, a glutathione S-transferase conjugated ubiquitin or a biotinylated ubiquitin. Using assays well known in the art, the presence of the label, and thus the amount of derivatized ubiquitination, can be identified.

Ubiquitination results in an increase in the molecular weight of the IR-β polypeptide. Accordingly, any assay that determines the molecular weight of the IR-β polypeptide, such as, for example, SDS-polyacrylamide gel electrophoresis, mass spectrometry or a variant thereof (MALDI, MALDI-TOF, MS/MS, etc), can be used to measure ubiquitination. High throughput procedures, such as, for example, MS/MS and MALDI-TOF are particularly preferred.

The ubiquitination assay is readily adapted to the large scale screening of compound libraries by converting it to a solid phase format. In one specific non-limiting example, ubiquitination assays can be performed with an appropriately engineered chimeric IR-β protein immobilized on a microtiter plate, in the presence of a derivatized ubiquitin. A “chimeric IR-protein” in this context is an IR-β precursor polypeptide comprising a heterologous peptide or polypeptide. Preferred heterologous peptides in this context include a myc epitope-hexahistidine tag positioned at the C-terminus of IR-β (i.e., IR-β-myc-His₆).

In one embodiment of high throughput screening for ubiquitination of IR-β, components of the reaction mixture are contacted, and aliquots of the ubiquitination assays are transferred to a microtiter plate with an appropriate surface to permit binding of IR-β protein thereto. Preferably, reactions are transferred to a microtiter plate whose wells have been coated with anti-IR-β antibody or anti-myc antibody. After washing away unbound proteins, the substrate-coated wells are directly imaged (e.g., for reactions performed with fluorescent or radio-labeled ubiquitin). The assay may be conducted in replica plates e.g., in the presence or absence of a series of compounds being tested.

In an alternative embodiment, ubiquitination of the IR-β substrate in the presence of a compound is determined using a cell-based assay. The only requirement for such an assay is that the compound being tested is capable of entering the cell. Accordingly, such an assay is particularly useful for identifying peptidyl and nucleic acid modulatory compounds that can be efficiently expressed following transfection or transduction into cells e.g., ribozymes, antisense, siRNA, shRNA, dominant negative modulatory. Such assays may also be amenable for determining the efficacy of antibodies or small organic molecules. This embodiment is based upon the finding that detectable c-Cbl-mediated ubiquitination of IR-β precursor occurs in CHO.T cells expressing myc-tagged APS (Ahmed et al., FEBS Letts 475, 31-34, 2000).

In one embodiment, cells capable of expressing APS, c-Cbl and IR-β precursor are incubated in the presence ubiquitin or a derivatized ubiquitin and a chemical compound to be tested for a time and under conditions sufficient for c-Cbl-mediated ubiquitination of IR-β precursor to occur, and the amount of ubiquitin bound to the IR-β precursor is determined.

In contrast to the cell-free system described supra, specific detection of ubiquitinated IR-β precursor is required, because other proteins will be ubiquitinated in the cells. Accordingly, it is particularly preferred to determine the amount of ubiquitin bound to IR-β precursor by first capturing the insulin receptor holoprotein or IR-β precursor polypeptide with an antibody (e.g., anti-insulin receptor antibody 83-14, Abeam, Inc, Cambridge Mass. 02139, USA; or specific anti-IR-β antibody as supplied e.g., by Lab Vision Corp., Fremont Calif. 94539, USA) and then detecting the amount of ubiquitin bound thereto using a second antibody that specifically binds ubiquitin (Santa Cruz Biotechnology, Santa Cruz, Calif., USA). As with the cell-free assay system supra, a derivatized ubiquitin may be imaged directly.

In a particularly preferred embodiment, antibodies that bind the insulin receptor are immobilized on a solid substrate, e.g., wells of a microtiter plate and contacted with cell extracts for a time and under conditions sufficient for an antigen-antibody complex to form thereby capturing the insulin receptor. After washing away unbound proteins, the wells are either directly imaged (e.g., for reactions performed with fluorescent or radio-labeled ubiquitin) or contacted with anti-ubiquitin antibody for a time and under conditions sufficient for an antigen-antibody complex to form. Binding of the anti-ubiquitin antibody is detected using a second antibody conjugated to horse-radish peroxidase enzyme or other detectable label.

The assay may be conducted in replica plates e.g., in the presence or absence of a series of compounds being tested to facilitate comparison of the amount of ubiquitination of the receptor in the presence and absence of the compound being tested.

In a further embodiment, a first replica plate is contacted with second antibody after capturing the insulin receptor and a second replica plate is contacted with a second antibody after binding of the anti-ubiquitin antibody. In accordance with this embodiment, measurement of the ratio of the amount of second antibody bound to the first and second replica plates indicates the amount of ubiquitinated insulin receptor relative to the total amount of insulin receptor in the cell or sample. Such measurement is particularly useful for correcting for sample variations. To facilitate high throughput analyses, it is particularly preferred for the second antibodies to be labelled with different colored dyes or fluorophores to permit their simultaneous detection.

Methods of Modifying Glucose Uptake

The present invention also provides a method of identifying a compound that reduces glucose uptake, said method comprising: (a) administering a compound to a genetically modified non-human animal comprising a genetic modification within an allele of its Cbl locus wherein said genetic modification reduces or prevents expression of a functional endogenous Cbl in said animal; and (b) determining the glucose uptake into liver, fat or muscle cells of the animal, wherein enhanced uptake compared to the glucose uptake into liver, fat or muscle cells of a Cbl-deficient animal to which the compound has not been administered indicates that the compound reduces glucose uptake.

In an alternative embodiment, the invention provides a method of identifying a compound that reduces glucose uptake into liver, fat or muscle cells, such as, for example, in the treatment of obesity, said method comprising: (a) administering a compound that enhances glucose uptake to a genetically modified non-human animal comprising a genetic modification within an allele of its Cbl locus wherein said genetic modification reduces or prevents expression of a functional endogenous Cbl in said animal and determining the glucose uptake into liver, fat or muscle cells; (b) administering a compound to the animal and determining the glucose uptake into liver, fat or muscle cells of the animal, wherein a similar or reduced uptake at (b) compared to (a) indicates that the compound reduces glucose uptake into liver, fat or muscle cells. Preferably, the animal is maintained on a diet comprising high glycemic index food, such as, for example, carrots or food supplemented with sucrose.

Preferably, the compound that enhances glucose uptake into liver, fat or muscle cells acts via a Cbl-mediated mechanism, which can be verified using the genetically modified Cbl-deficient animal, such as by determining the ability of the compound to enhance glucose uptake into liver, fat or muscle cells of Cbl-deficient mice according to a method described herein. In this case, step (a) supra of administering a compound that reduces glucose uptake into liver, fat or muscle cells comprises (i) administering a compound to a genetically modified non-human animal comprising a genetic modification within an allele of its Cbl locus wherein said genetic modification reduces or prevents expression of a functional endogenous Cbl in said animal; and (ii) determining the glucose uptake into liver, fat or muscle cells of the animal, wherein enhanced glucose uptake into liver, fat or muscle cells of the animal compared to a Cbl-deficient animal to which the compound has not been administered indicates that the compound reduces glucose uptake into liver, fat or muscle cells.

In an alternative embodiment, the invention provides a method of identifying a compound that enhances glucose uptake into liver, fat or muscle cells comprising: (a) administering a compound to a non-human animal expressing a functional Cbl protein and determining the glucose uptake into liver, fat or muscle cells of the animal; (b) determining the glucose uptake into liver, fat or muscle cells of a genetically modified non-human animal comprising a genetic modification within an allele of its Cbl locus wherein said genetic modification reduces or prevents expression of a functional endogenous Cbl in said animal; and (c) comparing the glucose uptake into liver, fat or muscle cells of the animals at (a) and (b) wherein a comparable uptake between (a) and (b) indicates that the compound enhances glucose uptake into liver, fat or muscle cells. Preferably, the animals are maintained on similar or identical diets, more preferably, on diets comprising high glycemic index food.

The compounds contemplated herein include Cbl inhibitory compounds or antagonists of a biological function of Cbl. In one embodiment, the compound is selected from the group consisting of: a peptide, including a peptide derived from Cbl and capable of inhibiting, reducing or repressing a Cbl function, including binding to a protein selected from the group consisting of CAP, CrkII and C3G; a Cbl dominant-negative mutant; a non-Cbl peptide inhibitors of Cbl; an antibody or antibody fragment which binds to Cbl and inhibits a Cbl function; a small organic molecule, and nucleic acid, including nucleic acid encoding said peptide derived from Cbl or said non-Cbl peptide inhibitor, an antisense nucleic acid directed against Cbl-encoding mRNA, or an anti-Cbl ribozyme.

Means for determining glucose uptake are well known in the art. Preferably, the process is performed ex vivo using liver, fat or muscle cells that have been previously isolated from the animal. In one embodiment, the glucose uptake is basal glucose uptake (i.e. glucose uptake measured in the absence of exogenously administered insulin). In another embodiment, the glucose uptake is insulin-mediated glucose uptake (i.e. glucose uptake measured following administration of insulin). All embodiments of the invention described herein apply mutatis mutandis to both basal glucose uptake and insulin-mediated glucose uptake unless otherwise stated.

Therapeutics

The modulators identified using the methods described herein are useful for the therapeutic or prophylactic treatment of diseases associated with associated with Cbl function. In one embodiment, the compounds are used to treat a condition selected from the group consisting of: hyperglycemia, hyperinsulinemia, obesity, adult-onset obesity, non-insulin-dependent diabetes mellitus, type II diabetes, glucose intolerance, and hypertrophy or hyperplasia of the islets of Langerhans, atherosclerosis or heart disease. In another embodiment, the compounds are used for cosmetic purposes, for example, by bodybuilders or persons wishing to modify their weight or body content of fat or muscle.

Accordingly, another aspect of the invention provides a method comprising administering an effective amount of a Cbl antagonist to an animal or human subject to inhibit or reduce the expression or activity of Cbl or a downstream effector of Cbl in the subject. By “downstream effector” is meant a gene or protein that is regulated by Cbl and that is associated with Cbl-mediated whole body insulin action and/or Cbl-mediated whole body energy expenditure e.g., pACC, pAMPK, IR, etc.

Preferably, the subject is a subject in need of treatment, such as a subject suffering from a condition selected from the group consisting of: elevated glucose uptake, reduced appetite or dietary intake, hyperglycemia, hyperinsulinemia, enhanced fat deposition or obesity, adult-onset obesity, non-insulin-dependent diabetes mellitus, type II diabetes, glucose intolerance, and hypertrophy or hyperplasia of the islets of Langerhans, atherosclerosis or heart disease.

To determine an appropriate dosage for treatment, data from the cell culture assays or animal studies are used, wherein a suitable dose is within a range of circulating concentrations that include the ED₅₀ of the active compound with little or no toxicity. The dosage may vary within this range depending upon the dosage form employed and the route of administration utilized. For any Cbl inhibitor or antagonist used in the method of the invention, the therapeutically effective dose can be estimated initially from cell culture assays. A dose may be formulated in animal models (e.g. any one or more of the mouse models described supra having genetic obesity-diabetes syndromes, such as hyperglycemia, hyperinsulinemia, atherosclerosis or heart disease) to achieve a circulating plasma concentration range that includes the IC₅₀ (i.e., the concentration of the test Cbl inhibitor which achieves a half-maximal inhibition of symptoms) as determined in cell culture. Such information can be used to more accurately determine useful doses in humans. Levels in plasma maybe measured, for example, by high performance liquid chromatography.

In an alternative embodiment, the present invention provides for the use of a Cbl antagonist in the preparation of a medicament for the treatment of a condition selected from the group consisting of: elevated glucose uptake, reduced appetite or dietary intake, hyperglycemia, hyperinsulinemia, enhanced fat deposition or obesity, adult-onset obesity, non-insulin-dependent diabetes mellitus, type II diabetes, glucose intolerance, and hypertrophy or hyperplasia of the islets of Langerhans, atherosclerosis or heart disease.

The present invention also provides a method of modulating the level of phosphorylated acetyl-CoA carboxylase (ACC) and/or ACC activity and/or AMPK activity and/or the level of phosphorylated AMPK in a cell or subject comprising modulating the activity or amount of a Cbl protein. As ACC and AMPK activities regulate fatty acid synthesis/oxidation and ATP synthesis, this method clearly extends to a method of modulating fatty acid synthesis and/or fatty acid oxidation and/or ATP synthesis. Preferably, the method is used to treat a subject suffering from a condition associated with aberrant insulin-action.

The present invention is further described with reference to the following examples and the accompanying drawings.

### EXAMPLE 1

### Phenotype of Cbl-Deficient Mouse

Methods

Animals.

c-Cbl^{-/-} mice were generated as described by Murphy et al., Mol. Cell Biol. 18, 4872-4882, 1998. Experiments were performed on mice maintained on the hybrid 129/SvJ C57BL/6 background. The animals were bred and housed in cages and kept on a 12-hour light/dark cycle with free access to food and water. Food intake and body weight were monitored on a weekly basis. All the experiments were carried out with the approval of the Garvan Institute/St. Vincent's Hospital Animal Experimentation Ethics Committee, following guidelines issued by the National Health and Medical Research Council.

Metabolic Assays.

Glucose (2 g/kg glucose i.p.) and insulin (0.5 U/kg insulin i.p.) tolerance tests were performed in overnight-fasted 16- to 18-week-old mice. Glucose (2 g/kg body weight) was administered into the intraperitoneal cavity following removal of a blood sample to test basal glucose concentration. Blood samples were taken at 15 min intervals for the following 90 min. Glucose assay was performed using the glucose oxidase method. Glucose levels were measured using a glucometer (ACCU-CHEK II; Roche Diagnostics Corp.). Insulin concentrations were measured using an ultrasensitive ELISA kit (Mercodia AB). Clearance of the glucose analogue [³H]-2-deoxyglucose (i.e., [³H]-2-DOG; 2 g/kg glucose i.p., 10 μCi/animal [³H]-2-DOG) into glucose-6-phosphate and glycogen in indicated tissues was measured as described by Cooney et al., EMBO J 23, 582-593, 2004. Other plasma measurements were performed on blood collected from the chest cavity into tubes containing EDTA and centrifuged at 14,000 g for 10 minutes to obtain the plasma. Concentrations of leptin, adiponectin (Linco Research Inc.) and thyroid hormones (total T3 and T4; ICN Pharmaceuticals Inc.) were assayed by RIA. Plasma triglycerides, glycerol, and non-esterified fatty acids were assayed using calorimetric kits (Roche Diagnostics Corp., Sigma-Aldrich and Wako Pure Chemical Industries Ltd., respectively).

Ex Vivo Glucose Uptake.

For determining glucose transport in muscle and adipose tissue, animals were fasted overnight and whole soleus muscle or gonadal white adipose tissue explants were incubated in vitro in the absence or presence of insulin in a Krebs Ringer buffer. Glucose uptake in isolated muscles from wild-type (WT) and c-Cbl^{-/-} mice was assayed in sealed conical flasks containing pre-gassed (95% O₂/5% CO₂) Krebs-Henseleit bicarbonate buffer, pH 7.4, supplemented with 2 mM sodium pyruvate, 8 mM mannitol, and 0.1% (w/v) BSA at 30° C. Muscles were allowed to recover for 10 minutes and incubated without or with insulin for 30 minutes. Glucose uptake was assayed for 16 minutes using [³H]-2-DOG (1 mM; 0.128 μCi/ml) as described by Viollet et al., J. Clin, Invest 111, 91-98, 2003. Glucose transport assays on fat explants were performed as described by Cooney et al., EMBO J 23, 582-593, 2004.

Adipocyte Size Determination.

Isolated adipocytes were obtained from excised epididymal fat pads by the collagenase method (Rodbell et al., J. Biol. Chem 239, 375-380, 1964). Cells were then fixed with 2% OsO₄ in PBS overnight at 37° C. Adipocyte diameter from 100-200 cells was determined using Photoshop software (Adobe Systems Inc.) from images of adipocyte suspensions obtained by light microscopy.

Ambulatory Activity.

Spontaneous ambulatory activity of individually-housed mice was evaluated over a 24-hour period using acrylic glass chambers (31 cm×18 cm×18 cm) and an Opto-Varimex-3 sensor system (Columbus Instruments). Consecutive photo beam breaks occurring in adjacent photo beams were scored as an ambulatory movement. Cumulative ambulatory activity counts were recorded every 20 minutes throughout the light/dark cycles. During the study, mice had ad libitum access to food and water.

Resting Metabolic Rate.

A custom-built open-circuit respirometer was used to measure rates of oxygen consumption (VO₂) and carbon dioxide production (VCO₂) in mice at a controlled temperature of 25° C. (Withers et al., Aust. J. Zool. 48, 241-258, 2000). Briefly, air was drawn through the chamber (500 ml total volume) at 400 ml/min. Expired air was dried and then passed through a Qubit S152 infrared CO₂ analyzer (Qubit Systems Inc.) and a Servomex 0A 184 paramagnetic O₂ analyzer (Servomex Group Ltd.). Baseline values of background O₂ and CO₂ were established for at least 15 minutes before and after each assessment. All measurements were performed during the light phase between 10 am and 4 pm and control and c-Cbl^{-/-} mice were assessed alternately. All animals were assessed for at least 2 hours under constant observation after a 30-minute acclimation period in the chamber. In view of the small chamber volume, all animals remained sedentary during the recording period. Resting VO₂ and VCO₂ were determined using a custom Visual Basic program (Withers et al., Aust. J. Zool. 48, 241-258, 2000). Body temperature was measured using a rectal probe (BAT-10 Digital Thermometer; Physiotemp Instruments Inc.).

Determination of Body Composition.

Fat and lean body mass was measured using dual-energy x-ray absorptiometry (Lunar PIXImus2 mouse densitometer; GEHealthcare) in accordance with the manufacturer's instructions.

In Situ Hybridization.

Coronal slices (20 μm) of fresh-frozen brains were cut and thaw-mounted on charged slides. For in situ hybridization, the following DNA oligonucleotides complementary to the following mRNAs were labelled with [³⁵S]-thio-dATP (Amersham Biosciences Ltd. or NEN Life Science Products) using terminal deoxynucleotidyl transferase (Roche Diagnostics Corp. or Amersham Biosciences Ltd.):

1. mouse neuropeptide Y mRNA probe:

(SEQ ID NO: 264)

5′-GAGGGTCAGTCCACACAGCCCCATTCGCTTGTTACCTAGCAT-3′;

2. mouse cocaine- and amphetamine-regulated transcript mRNA probe:

(SEQ ID NO: 265)

5′-TCCTTCTCGTGGGACGCATCATCCACGGCAGAGTAGATGTCCAG
G-3′;

3. mouse corticotropin-releasing hormone mRNA probe:

(SEQ ID NO: 266)

5′-CCGATAATCTCCATCAGTTTCCTGTTGCTGTGAGCTTGCTGAGC
T-3′;
and

4. mouse thyrotropin-releasing hormone mRNA probe:

(SEQ ID NO: 267)

5′-AACCTTACTCCTCCAGAGGTTCCCTGACCCAGGCTTCCAGTTGT
G-3′.

Matching sections from the same portion of the hypothalamus of knockout and control mice were assayed together, following the method of Young Methods Enzymol. 168, 702-710, 1989, using variations as described by Tsunashima et al., Neuroscience 80, 1019-1032, 1997 and Schalling et al., Neuroscience 24, 337-349, 1988. The mRNA levels in samples were evaluated by measurement of silver grain densities over individual neurons from photoemulsion-dipped sections.
Tissue Processing and Immunoblotting.

Tissues were rapidly removed from fed wild-type and c-Cbl^{-/-} mice, frozen in liquid nitrogen, powdered, resuspended in RIPA buffer (PBS, pH 7.5; 1% NP-40; 0.5% sodium deoxycholate; 0.1% SDS) supplemented with protease and phosphatase inhibitors (10 μg/ml phenylmethylsulfonyl fluoride, 10 μg/ml aprotinin, 10 μg/ml leupeptin, 1 mM. Na₃VO₄, 10 mM NaF, 10 mM sodium pyrophosphate), and solubilized for 2 hours at 4° C. Aliquots of tissue homogenates were resolved by SDS/PAGE and immunoblotted with publicly-available antibodies against GLUT4 (Robinson et al., J. Cell Biol. 117, 1181-1196, 1992); the β subunit of the IR and insulin receptor substrate-1 (Santa Cruz Biotechnology Inc.); syntaxin-4 (Tellam et al., J. Biol. Chem. 272, 6179-6186, 1997); UCP3 (Affinity Bioreagents Inc.); AMPKα and pThr172-AMPK, and pSer79-ACC (Cell Signaling Technology Inc.); ACC (Upstate Biotechnology Inc.); PGC1α (Chemicon International Inc.); and PDGFRβ (Genzyme Corp.). Quantitation of immunolabeled bands was performed using a VersaDoc Imaging System (BioRad Laboratories).

Electron Microscopy.

Muscles were removed immediately after euthanasia and cut into slices while immersed in modified Karnovsky's fixative (2.5% glutaraldehyde/4% paraformaldehyde in 1 M cacodylate buffer, pH 7.4). Samples were further fixed overnight in the same fixative and post-fixed with 2% osmium tetroxide, dehydrated through an ascending series of ethanol, and embedded in Spurr's epoxy resin. Ultrathin sections were cut with a Reichert-Jung ultramicrotome, double-contrasted with uranyl acetate and lead citrate, and viewed and photographed with a Philips CM120BioTwin transmission electron microscope. Mitochondrial size and cristae density of sub sarcolemmal mitochondria from matching sections of muscles from both wild-type and c-Cbl^{-/-} mice were determined as described by Schwerzmann et al., Proc. Natl Acad. Sci. 86, 1583-1587, 1989, using Axiovision software (Carl Zeiss International).

Statistical Analysis.

Data are presented as mean ±SEM, and statistical analysis was performed using an unpaired Student's t test. Differences at P<0.05 were considered to be statistically significant.

Results

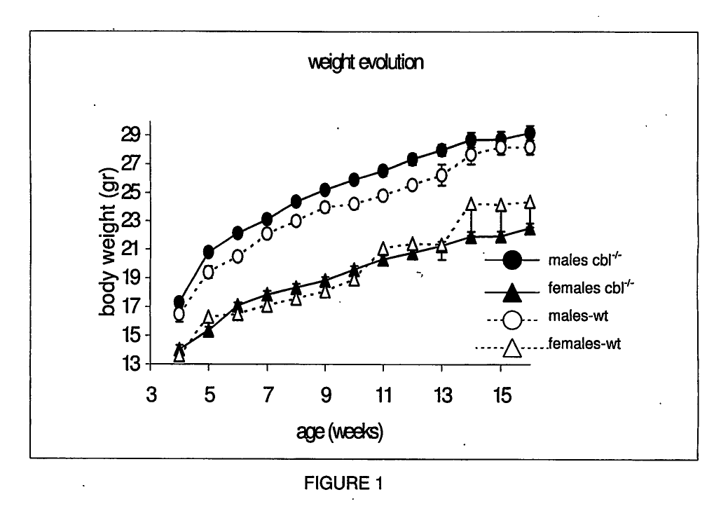

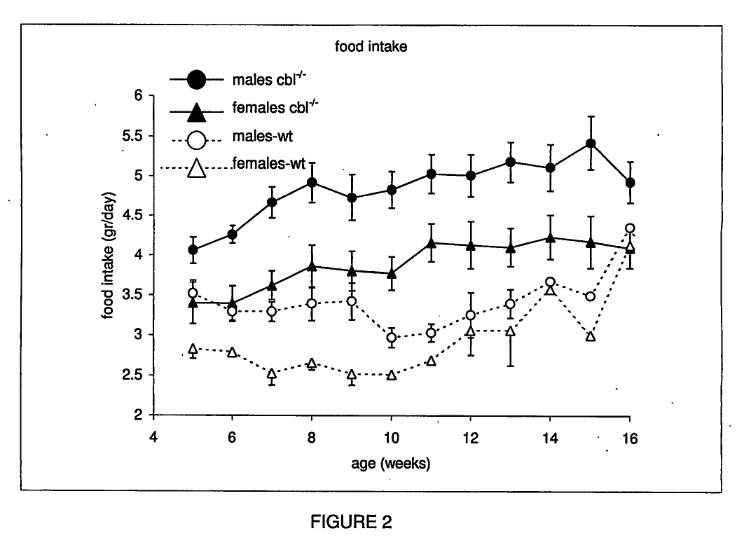

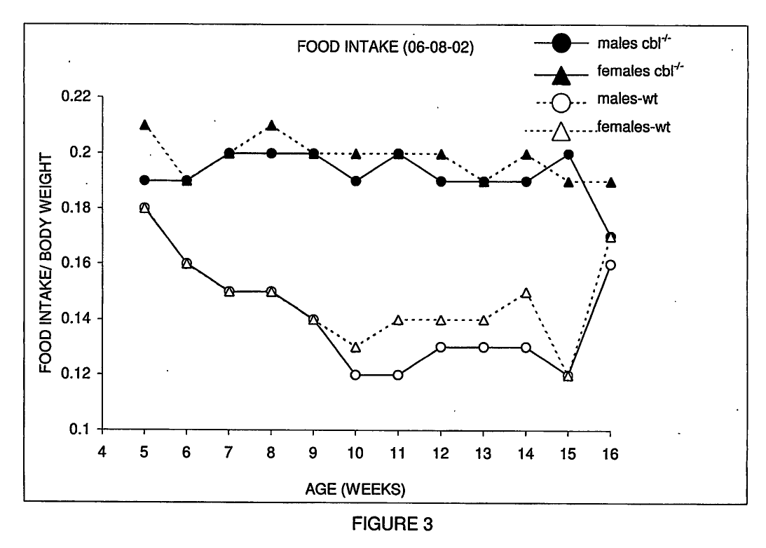

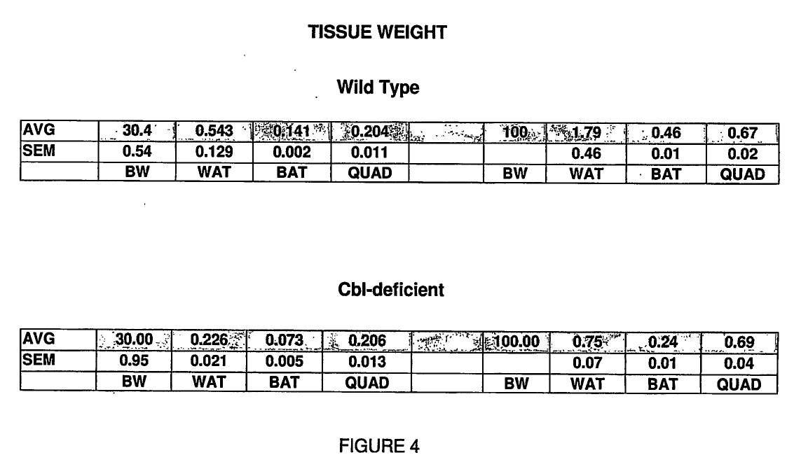

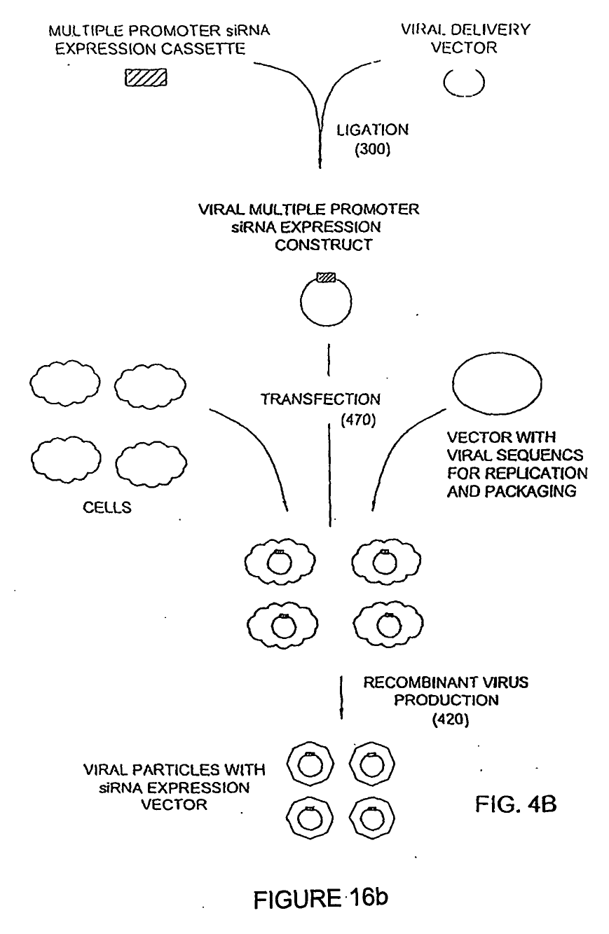

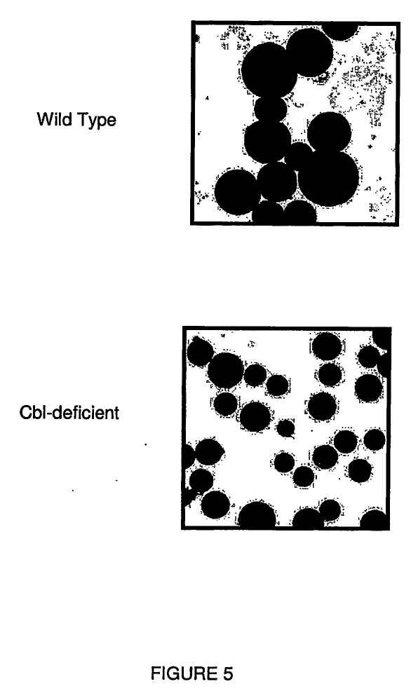

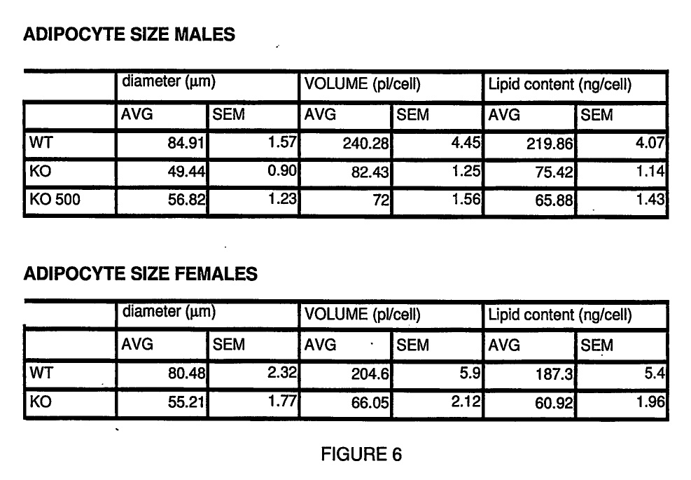

The metabolic and physiological phenotype associated with a deficit in Cbl expression is summarized in Table 1. FIGS. 1 through 6 show that Cbl-deficient mice have significantly higher body weight and dietary intake, however reduced fat deposition and smaller adipocytes, compared to otherwise isogenic non-mutant animals of the same gender. The body temperature of Cbl-deficient animals is also enhanced (Tables 1 and 2) and, in two independent experiments, the average body temperature of Cbl-deficient males and females increased (Table 2). This difference in body temperature was observed independently of the time of day and of whether the animals were fed or fasted overnight (data not shown). There was no significant difference in the circulating levels of triiodothyronine (T3) or thyroxine (T4) between c-Cbl^{-/-} and wild-type mice in either the fed or the fasted state (Table 1), which suggests that increased thermogenesis in these animals is unlikely due to changes in the thyroid axis.

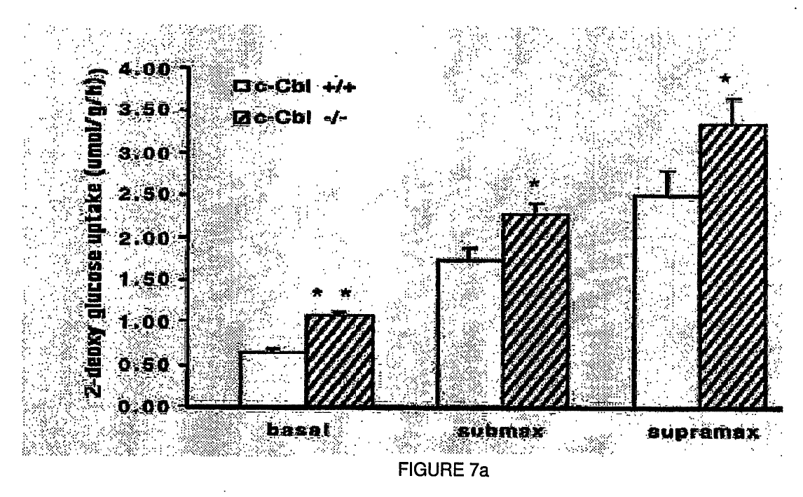

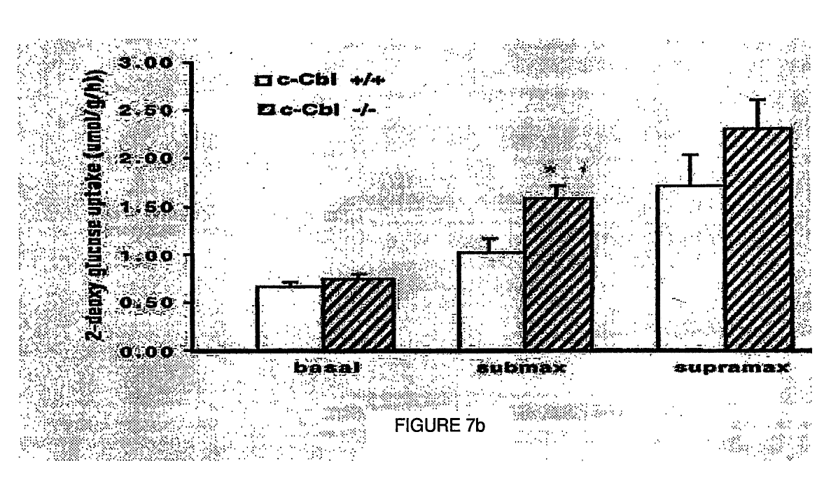

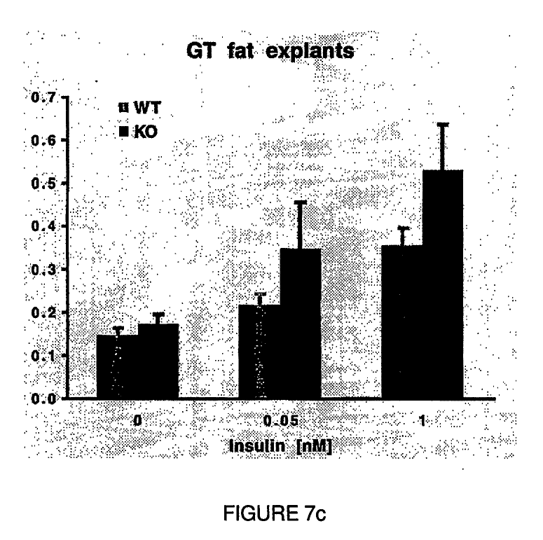

In contrast to what was expected, disruption of the Cbl gene leads to enhanced basal glucose uptake into both adipocytes and muscle cells (FIGS. 7a-7c). As shown in FIG. 7 the rate of 2-deoxyglucose uptake was significantly upregulated in SOL isolated from c-CBL^{-/-} mice compared to c-CBL^{+/+} controls, indicating an enhanced metabolic rate in c-CBL deficient mice. In the presence of 300 μM/ml insulin (submax), the amount of 2-deoxyglucose uptake was significantly increased in both SOL and EDL isolated from c-CBL^{-/-} mice compared to c-CBL^{+/+} mice. When exposed to sufficient levels of insulin to ensure maximal insulin-stimulated glucose transport (ie. 1000 μU/ml insulin) there was a significant increase in 2-deoxyglucose transport in SOL isolated from c-CBL^{-/-} mice compared to c-CBL^{+/+} mice. These results indicate that c-CBL^{-/-} mice have increased insulin-stimulated glucose uptake (approximately 30% increase) in isolated SOL and EDL muscles, thereby indicating that c-CBL^{-/-} mice have increased peripheral insulin sensitivity. As shown in FIG. 7c the amount of 2-deoxyglucose incorporated into fat explants from c-CBL^{-/-} (KO) mice was increased in the presence of both 0.05 nM and 0.1 nM of insulin compared to c-CBL^{+/+} (WT) mice. These data indicate that c-CBL^{-/-} mice have increased insulin-stimulated glucose uptake in fat explants compared to c-CBL^{+/+} mice, indicating increased peripheral insulin sensitivity in knockout mice.

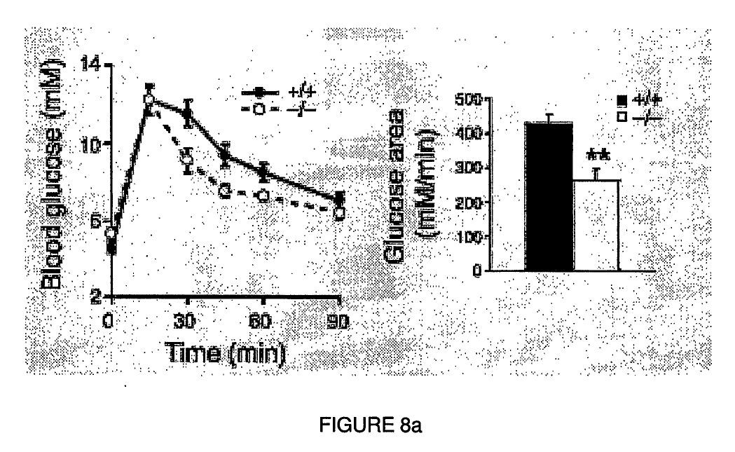

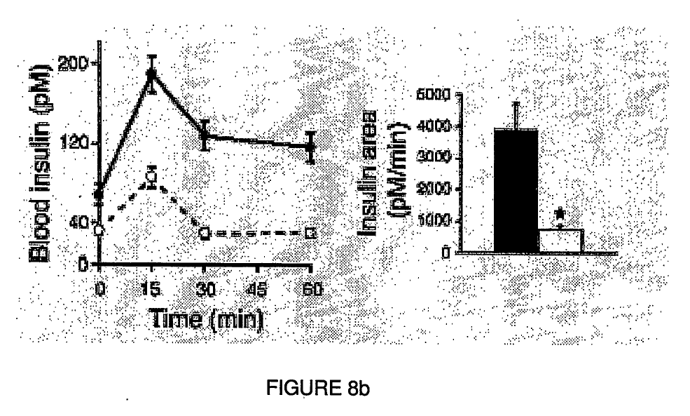

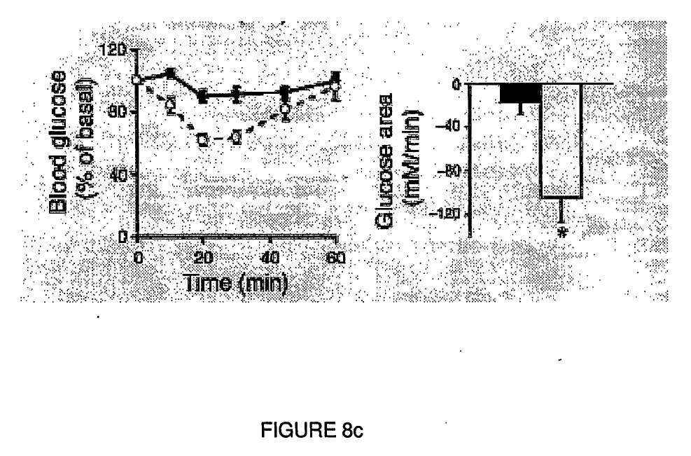

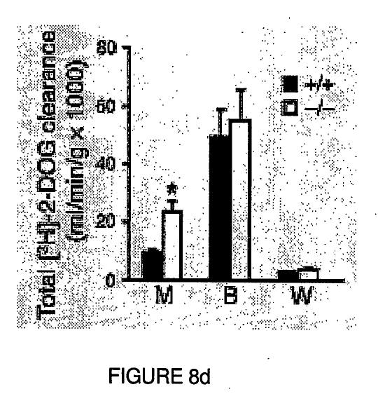

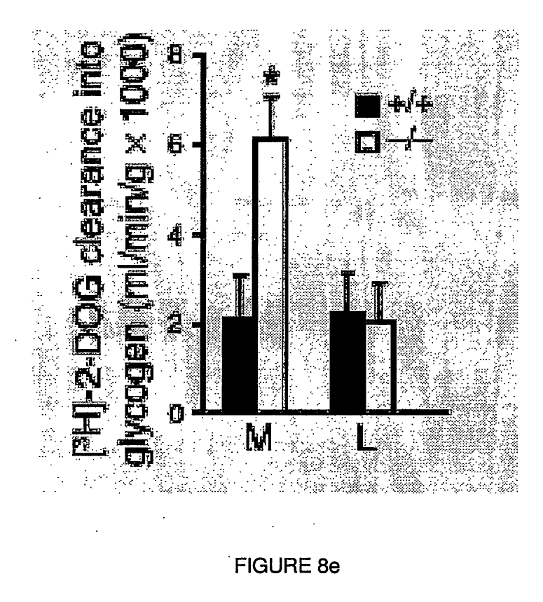

The c-Cbl^{-/-} mice also displayed improved glucose tolerance compared with otherwise isogenic wild-type animals (FIG. 8a). Moreover, this effect was observed in the face of markedly reduced insulin levels throughout the glucose tolerance test in c-Cbl^{-/-} mice (FIG. 8b). The increased blood glucose clearance in the presence of lower circulating insulin levels indicates that peripheral insulin action is enhanced in c-Cbl^{-/-} animals. To examine insulin action independently of pancreatic function, insulin tolerance tests were performed on wild-type and c-Cbl^{-/-} animals. As shown in FIG. 8c, after administration of a low dose of insulin (0.5 U/kg), c-Cbl^{-/-} animals exhibited a greater decline in blood glucose levels compared with wild-type mice.

To determine the contribution of different tissues to the improved glucose tolerance observed in c-Cbl^{-/-} mice, the inventors examined the clearance of [³H]-2-deoxyglucose ([3H]-2-DOG) co-administered with the glucose load during the glucose tolerance test. As shown in FIG. 8d, total [³H]-2-DOG clearance during the glucose tolerance test was 2-fold higher in skeletal muscle of c-Cbl^{-/-} mice compared with wild-type animals. There was no significant difference in [³H]-2-DOG uptake in white or brown adipose tissue between c-Cbl^{-/-} and wild-type animals during the same experiments. The inventors also examined the clearance of [³H]-2-DOG into glycogen in muscle and liver. In muscle from c-Cbl^{-/-} mice, [³H]-2-DOG clearance into glycogen was increased compared with that in muscle from wild-type animals. This is consistent with the total [³H]-2-DOG clearance data (FIG. 8e). However, there was no significant difference in [³H]-2-DOG clearance into glycogen in liver in the c-Cbl^{-/-} mice compared with controls.

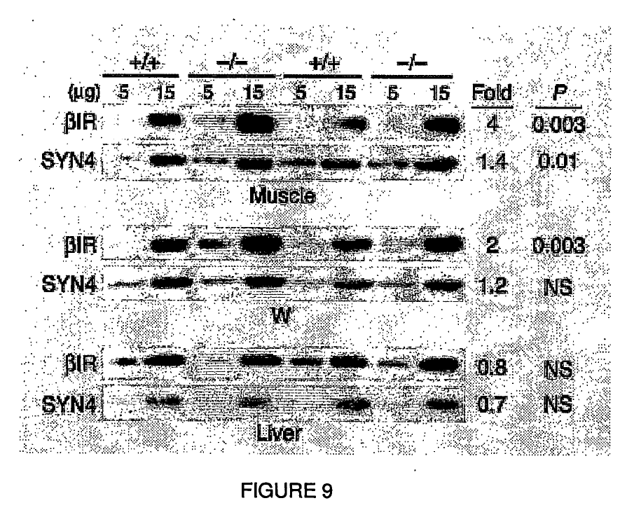

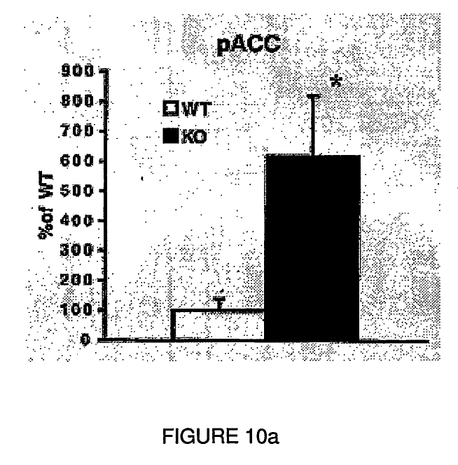

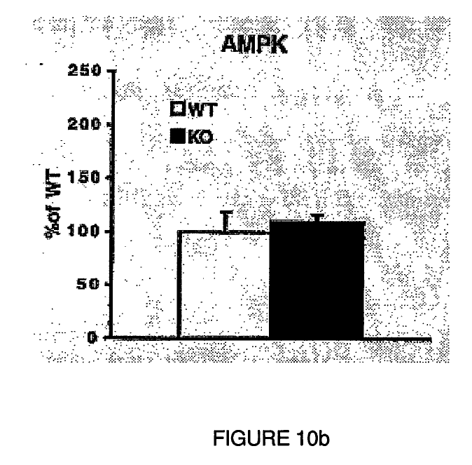

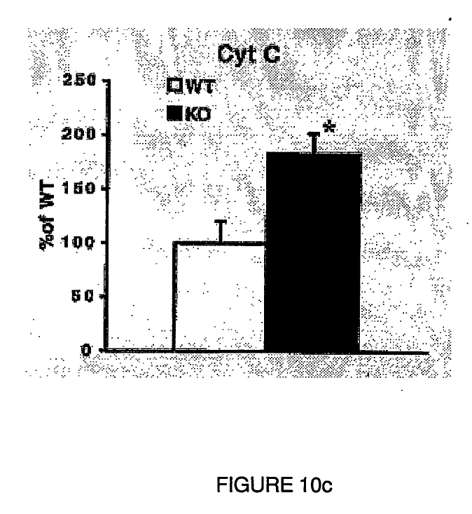

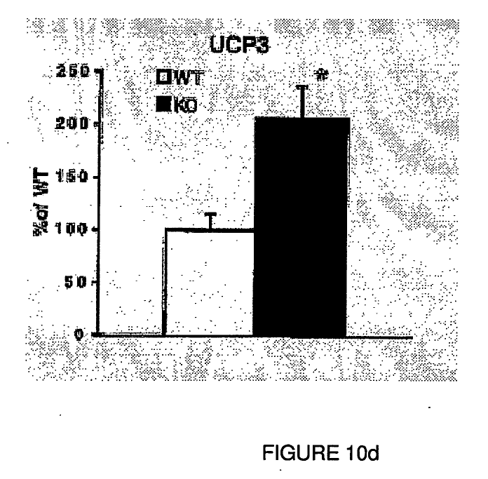

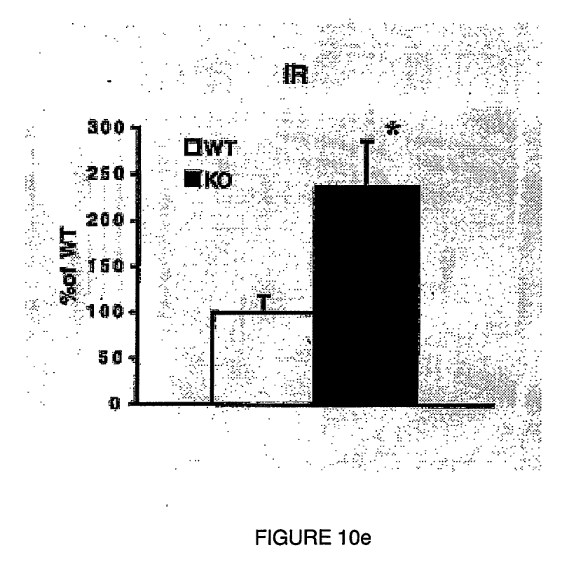

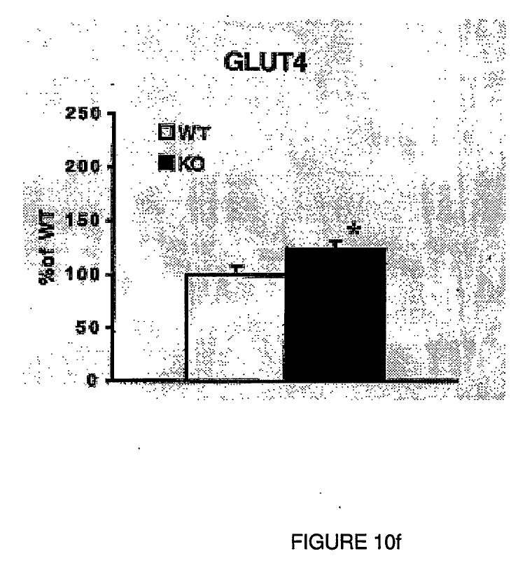

The inventors also examined whether the total protein levels of the IR and other proteins involved in insulin action were changed in muscle tissue from c-Cbl^{-/-} mice. As shown in FIGS. 9 and 10e, IR protein levels were increased 4-fold in muscle from c-Cbl^{-/-} mice compared with wild-type animals. In adipose tissue the inventors observed only a 2-fold increase in IR protein levels in c-Cbl^{-/-} mice, whereas in liver the IR level was not significantly altered (FIG. 9). The levels of insulin receptor substrate-1 (not shown) were marginally increased in muscle from c-Cbl^{-/-} deficient animals, whereas GLUT4 (FIG. 10f) levels were not significantly different. As a control, the inventors immunoblotted all samples with an antibody specific for syntaxin-4 in all 3 tissues from wild-type and c-Cbl^{-/-} mice.

The inventors next assessed circulating levels of adipokines known to regulate muscle metabolism. The inventors did not observe any change in fasting adiponectin levels; however, there was a significant decrease (66%) in circulating leptin levels in the c-Cbl^{-/-} animals compared with controls (Table 1). To examine the role of hypoleptinemia in the hyperphagia observed in c-Cbl^{-/-} mice, the inventors measured mRNA levels of relevant neuropeptides in the hypothalamus and found that the mRNA levels of the orexigenic factor neuropeptide Y in the arcuate nucleus of c-Cbl^{-/-} mice was increased (55%, P<0.05, n=5) compared with those in wild-type animals. The inventors also observed a significant decrease (approximately 30%, P<0.05, n=5) in the expression of the anorexigenic factor cocaine and amphetamine-regulated transcript in c-Cbl^{-/-} mice compared with controls. The inventors did not observe any significant change in mRNA levels of corticotropin-releasing hormone and thyrotropin-releasing hormone in neurons of the paraventricular nucleus (data not shown). These changes suggest that the hyperphagia observed in c-Cbl^{-/-} mice may be secondary to the lower leptin levels and decreased adiposity.

To further confirm the increased energy expenditure in c-Cbl^{-/-} mice, the inventors examined the metabolic rate of these animals by indirect calorimetry using a custom built system specifically designed to measure oxygen consumption and carbon dioxide production in small animals. Whole-body oxygen consumption was increased by 22% in c-Cbl—deficient mice compared with wild-type animals after correction for the difference in lean mass between the 2 groups (Table 1). The respiratory quotient was significantly reduced in c-Cbl^{-/-} mice (Table 1), which suggests that the increased energy expenditure was supported by increased fatty acid oxidation. Consistent with this, the inventors observed significantly lower plasma FFA levels in c-Cbl^{-/-} mice compared with control animals (Table 1). In addition, triglyceride levels in the liver of c-Cbl^{-/-} mice were significantly lower (5.3±0.28 μmol/g tissue, n=10, P<0.01) than those in control animals (9.65±1.31 μmol/g tissue, n=10), whereas triglyceride levels in the quadriceps muscle from wild-type and Cbl^{-/-} mice were not statistically different (8.5±0.8 μmol/g tissue, n=9, versus 6.7±0.6 μmol/g tissue, n=7, respectively). A significant increase in ambulatory activity was also observed in Cbl^{-/-} mice, which would also contribute to increased energy expenditure (Table 1). This increased activity was particularly evident in the dark phase (approximately 4-fold higher than in wild-type mice), suggesting increased food-seeking behavior consistent with the hyperphagia.

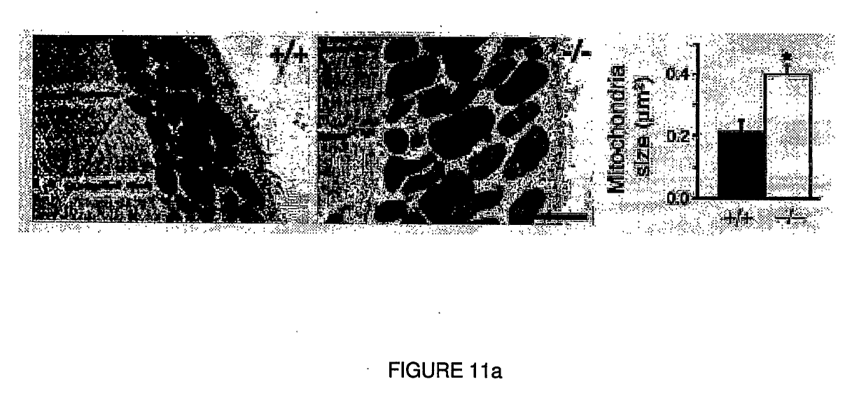

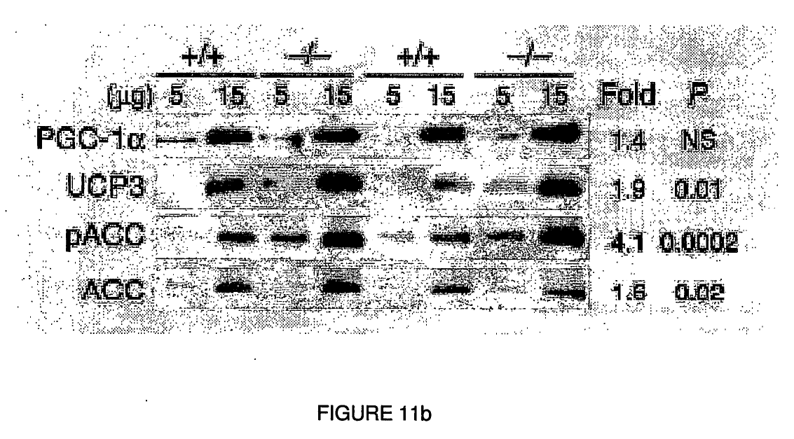

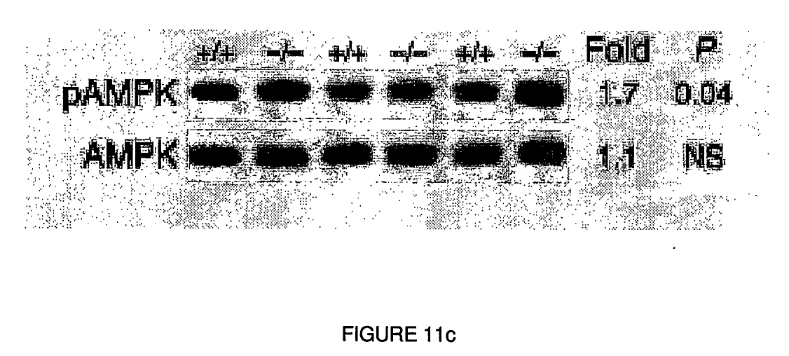

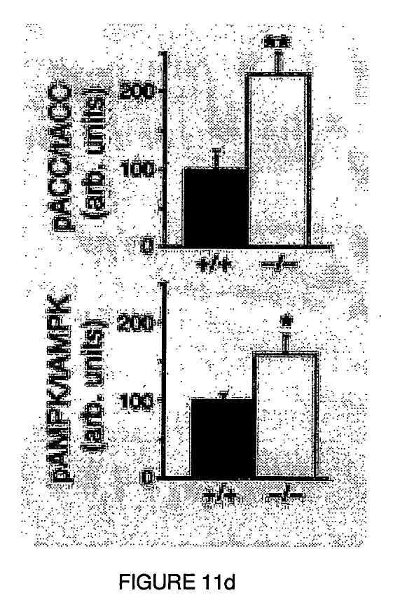

To explore a possible mechanism for the increased whole-body energy expenditure, the inventors examined mitochondrial size and function in Cbl^{-/-} and wild-type mice. The inventors observed a 4-fold increase in acetyl-CoA carboxylase (ACC) phosphorylation in muscle from the c-Cbl^{-/-} mice (FIGS. 10a, 11b and 11d). ACC is a rate-limiting enzyme for mitochondrial FFA oxidation, which is inactivated by phosphorylation. The level of ACC was also increased moderately (1.6-fold, FIG. 11b), and that of uncoupling protein-3 (UCP3) was increased 1.9-fold (FIGS. 10d and 11b), in muscle from c-Cbl^{-/-} mice compared with controls. There was a 2-fold increase in the average size of sub-sarcolemmal mitochondria in c-Cbl^{-/-} mice compared with wild-type animals (FIG. 11a). However, the inventors did not observe any significant change in the mitochondrial area in the subsarcolemmal region, or in cristae density (Table 1). The inventors were also unable to detect any significant change in the level of the PGC1α protein in muscle from c-Cbl^{-/-} mice (FIG. 11b).

To explore the mechanism for increased ACC phosphorylation in muscle from c-Cbl^{-/-} mice, the inventors examined the expression and phosphorylation state of the upstream ACC regulator AMP-activated protein kinase (AMPK). Consistent with an increase in AMPK activity and increased ACC phosphorylation, the inventors observed a significant 70% increase in AMPK phosphorylation in muscle from c-Cbl^{-/-} mice with no significant change in total expression (FIGS. 10c, 11c and 11d).

These data suggest that c-Cbl may be an important upstream regulator of AMPK activity in muscle, and that this effect may be a major contributor to enhanced energy expenditure in these animals.

These data also point to muscle mitochondrial function as a key therapeutic target for the treatment of type-2 diabetes, obesity, atherosclerosis or heart disease. Without being bound by any theory or mode of action, the data herein indicate that c-Cbl normally acts as a suppressor of the energy homeostat that regulates lean body mass, and overexpression of c-Cbl leads to increased body weight and insulin resistance. Mapping the molecular role of c-Cbl in energy homeostasis is a high priority for controlling body weight in humans in the face of increased food consumption.

### EXAMPLE 2

### In Vitro Validation of the Regulatory Role of c-Cbl on Phosphorylation of ACC and AMPK and in Regulating Insulin Receptor Levels in Isolated Myoblasts by RNAi Knockdown of c-Cbl

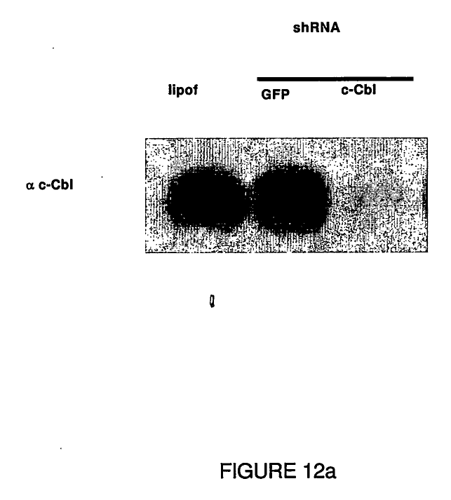

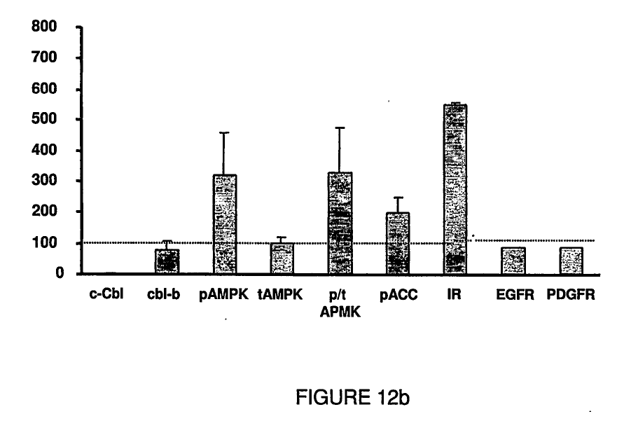

Murine C2C12 Cells were transfected with either no DNA or 15 mg of control vector DNA comprising shRNA that targets expression of green fluorescent protein (GFP) or alternatively c-Cbl. The shRNA against c-Cbl comprised the nucleotide sequence: 5-GACACTTTCCGGATTACTA-3′ (SEQ ID NO: 268) corresponding to nucleotides 619-637 of murine c-Cbl mRNA (NCBI Accession No. X57111; Blake and Langdon Oncogene 6, 653-657, 1991) and complementary sequence thereto separated by intervening loop. Cells were transfected using lipofectin and, after 3 days incubation, cells were harvested, homogenized, and cells extracts subjected to western blotting as described in Example 1 using antibodies against c-Cbl. Data presented in FIG. 12a indicate that c-Cbl expression is reduced more than 95% in mouse cultured C2C12 myoblasts. Reduced expression of c-cbl was only in the presence of shRNA against c-Cbl.

TABLE 1
Phenotype of Cbl-deficient (Cbl^{-/-}) mice

| | | | Change in |
|---|---|---|---|
| | | | Cbl^{-/-}relative |
| Parameter | c-Cbl^{+/+} (WT) | Cbl^{-/-} | to WT |
| Body weight (g) | 27.4 ± 0.4 | 28.7 ± 0.5 | None |
| Epididymal fat mass (g) | 0.45 ± 0.05 | 0.25 ± 0.02^{B} | Decreased |
| Inguinal subcutaneous fat mass (g) | 0.30 ± 0.04 | 0.14 ± 0.02^{B} | Decreased |
| Epididymal adipocyte size (pl) | 275 ± 16 | 98 ± 4^{B} | Decreased |
| Brown adipose mass (mg) | 116 ± 12 | 71 ± 2.8^{B} | Decreased |
| Liver mass (g) | 1.23 ± 0.06 | 1.32 ± 0.05 | None |
| Fat mass (% total by DXA) | 17.9 ± 2.1 | 11.5 ± 0.3^{A} | Decreased |
| Lean mass (% total by DXA) | 82.1 ± 2.1 | 88.4 ± 0.3^{A} | Increased |
| Fasting glucose (mM) | 8.9 ± 0.5 | 9.8 ± 0.5 | None |
| Fasting insulin (pM) | 68.2 ± 10 | 31.6 ± 3 | Decreased |
| Fasting leptin (ng · ml) | 3.82 ± 0.38 | 1.28 ± 0.36^{A} | Decreased |
| Fasting adiponectin (μg · ml) | 2.11 ± 0.14 | 1.98 ± 0.24^{B} | None |
| Fed plasma T3 (nM) | 1.14 ± 0.1 | 1.06 ± 0.1 | None |
| Fed plasma T4 (nM) | 31.3 ± 3.5 | 34.3 ± 2.6 | None |
| Fasting triglycerides (mM) | 0.9 ± 0.08 | 0.73 ± 0.06 | None |
| Fasting NE fatty acids (mM) | 0.83 ± 0.1 | 0.56 ± 0.05^{B} | Decreased |
| Fasting glycerol (mg/ml) | 0.77 ± 0.07 | 0.63 ± 0.07 | None |
| Average Body Temperature (° C.) | 37.0 ± 0.02 | 38.3 ± 0.1^{B} | Increased |
| VO₂ (ml/g lean body mass/hr) | 4.27 ± 0.16 | 5.24 ± 0.26^{B} | Increased |
| VCO₂ (ml/g lean body mass/hr) | 3.61 ± 0.13 | 4.18 ± 0.2^{A} | Increased |
| Respiratory Quotient (RQ) | 0.85 ± 0.01 | 0.8 ± 0.01^{A} | Decreased |
| Ambulatory activity in light phase(events 10⁻³) | 2,598 ± 515 | 5,596 ± 486^{A} | Increased |
| Ambulatory activity in dark phase(events 10⁻³) | 4,888 ± 1,035 | 18,337 ± 3,315^{B} | Increased |
| Food Intake | | | Increased |
| Glucose tolerance | | | Increased |
| Glucose transport into fat | | | Increased |
| Glucose transport into muscle | | | Increased |

Data represent the mean ± SEM of 8-20 different animals, except for the adipocyte size measurements for which 3 animals were used. A, P < 0.05; B, P < 0.01.
Abbreviations:
DXA, Dual energy X-ray absorptiometry;
VO₂, rate of oxygen consumption;
VCO₂, rate of carbon dioxide production.

TABLE 2
Enhanced body temperature in Cbl-deficient animals (Average ° C. ± SEM)

| | Males | | Females | |
|---|---|---|---|---|
| Experiment No. | Cbl^{+/+} | Cbl^{-/-} | Cbl^{+/+} | Cbl^{-/-} |
| 1 | 37.11° C. ± 0.26° C. | 38.93° C. ± 0.22° C. | 37.60° C. ± 0.30° C. | 38.06° C. ± 0.15° C. |
| 2 | 37.25° C. ± 0.16° C. | 38.17° C. ± 0.14° C. | 37.62° C. ± 0.09° C. | 37.31° C. ± 0.12° C. |

TABLE 3
Analysis of metabolic rate of cCBL^{-/-} mice

| | | VO2 | VCO2 | | | |
|---|---|---|---|---|---|---|
| | Tb (° C.) | (ml/g/h) | (ml/g/h) | RQ | conductance | |
| WT | 36.7 | 3.5 | 2.96 | 0.85 | 0.272 | mean |
| | 0.318 | 0.120 | 0.121 | 0.05 | 0.009 | se |
| | P < 0.02 | P < 0.001 | P < 0.002 | P < | P < 0.01 | |
| | | | | 0.04 | | |
| c-CBL | 37.6 | 4.6 | 3.7 | 0.8 | 0.36 | mean |
| KO | | | | | | |
| | 0.2 | 0.22 | 0.2 | 0.022 | 0.02 | se |

Cell extracts were also subjected to western blotting as described in Example 1, to determine the levels of Cbl-b, insulin receptor (IR), the mitochondrial proteins AMPK (tAMPK) and ACC, and the phosphorylated forms thereof, pAMPK and pACC. Data presented in FIG. 12b indicate that targetted knockdown of c-Cbl expression does not affect expression of Cbl-b or total AMPK level, but leads to a 5-fold increase in IR levels, and a 3-fold increase in pAMPK and 2-fold increase in pACC, thereby mimicking the phenotype seen in c-Cbl^{-/-} knockout animals. These data further validate c-Cbl as an important upstream regulator of both ACC and AMPK activity in regulating whole body energy expenditure, and validate muscle mitochondrial function, especially AMPK and ACC activities, as key therapeutic targets for the treatment of type 2 diabetes, obesity, atherosclerosis or heart disease.

Finally, comparable results for C2C12 cells and animal models validate the utility of C2C12 myoblasts for high-throughput compound screening to identify therapeutic agents.

### EXAMPLE 3

### Measurement of ACC and AMPK Enzyme Activities

Measurement of ACC Enzyme Activity

The CO₂ fixation technique (Kudo et al., Biochim Biophys Acta 1391, 67-75, 1996) is used to measure ACC activity e.g., in PEG fractions of cell lysates. Briefly, about 12.5 μg protein is added to a reaction mixture in a final volume of about 165 μl containing 60.6 mmol/l Tris acetate, 1 mg/ml BSA, 1.32 μmol/l ATP, 2.12 mmol/l β-mercaptoethanol, 5 mmol/1 magnesium acetate, 1.06 mmol/l acetyl CoA, 18.08 mmol/l NaH¹⁴CO₃, and 0 or 10 mmol/1 magnesium citrate. Samples are incubated at 37° C. for about 5 min, and the reactions are stopped by adding about 25 μl of 10% perchloric acid. Samples are spun at 3,500 rpm for 20 min, and then about 160 μl supernatant is placed in glass vials and dried under medium heat. The samples are resuspended in about 100 μl distilled deionized water (ddH₂O) and about 4 ml scintillation fluid and then counted for the presence of radiolabeled malonyl CoA. ACC activity is expressed as nanomoles of malonyl CoA produced per minute per milligram of protein.

Measurement of AMPK Enzyme Activity

AMPK activity e.g., in PEG fractions of cell lysates, is measured by following the incorporation of ³²P into the synthetic peptide SAMS (Kudo et al., Biochim Biophys Acta 1391, 67-75, 1996). The assay mixture contains about 40 mmol/l HEPES, pH 7.0, 80 mmol/l NaCl, 0.8 mmol/l EDTA, 1 mmol/l DTT, 0.2 mmol/l [γ-³²P]ATP, 5.0 mmol/l MgCl₂, 0.2 mmol/l SAMS, 8% glycerol, 0.01% Triton X-100, and about 2-5 μg of PEG precipitate, in the presence or absence of about 200 μmol/l AMP. Incorporation of ³²P into SAMS is measured at 30° C. for about 5-10 min. An aliquot of the reaction is then blotted onto phosphocellulose paper and washed four times in H₃PO₄ and once in acetone. The phosphocellulose is dried and counted in 4 ml scintillant. AMPK activity is expressed as picomoles of ³²P incorporated per minute per milligram of protein.

### EXAMPLE 4

### Immunodetection of ACC and AMPK Phosphorylation

Antibodies

Antibodies against AMPKα and pThr172-AMPK and pSer79-ACC are obtained from Cell Signaling Technology Inc. Antibodies against ACC are obtained from Upstate Biotechnology Inc.

Western Blotting

Sample Preparation

Tissues or cells in RIPA buffer (PBS, pH 7.5; 1% NP-40; 0.5% sodium deoxycholate; 0.1% SDS) supplemented with protease and phosphatase inhibitors (10 μg/ml phenylmethylsulfonyl fluoride, 10 μg/ml aprotinin, 10 μg/ml leupeptin, 1 mM Na₃VO₄, 10 mM NaF, 10 mM sodium pyrophosphate), and solubilized for 2 hours at 4° C.

Immunoblottinq

Aliquots of tissue homogenates are resolved by SDS/PAGE and immunoblotted according to standard procedures. Quantitation of immunolabeled bands was performed using a VersaDoc Imaging System (BioRad Laboratories).

High Throughput Assay Formats

Lysate Preparation

Cells are lysed for about 30 min at 4° C. with buffer containing 50 mM Tris-HCl, pH 8.0, 135 mN NaCl, 1% Triton X-100, 1.0 mM EDTA, 1.0 mM sodium pyrophosphate, 1.0 mM sodium orthovanadate, 10 mM NaF and protease inhibitors (Roche Diagnostics; 1 tablet per 7 ml buffer).

ELISA

To determine phosphorylation of ACC using this assay format, replica antigen plates are produced and probed separately with ACC and pSer79-ACC antibodies and the signal ratio of bound antibody determined. Generally, the ratio of pSer79-ACC/ACC antibody binding is determined.

To determine phosphorylation of AMPK using this assay format, replica antigen plates are produced and probed separately with AMPK and pThr172-AMPK antibodies and the signal ratio of bound antibody determined. Generally, the ratio of pThr172-AMPK/AMPK antibody binding is determined.

Thus, cell lysates are diluted in coating solution (50 mM sodium carbonate, pH 9.6; or 20 mM Tris-HCl, pH 8.5; or 10 mM PBS, pH 7.2) to a concentration of about 0.1-10 μg/ml protein. The diluted antigen is replica-adsorbed onto the wells of 96 or 256 well microtiter plates by adding 50-100 μl antigen in coating solution to the wells and incubating for about 1 hour at room temperature. The plates are emptied and then 300 μl blocking solution (1-10% (w/v) BSA in coating solution, or 1′-10% (w/v) nonfat dry milk in coating solution, or 1-10% (w/v) casein in coating solution, or 1-10% (w/v) gelatin in coating solution) is added to the wells. The plates are incubated again at room temperature for about 1 hour. The plates are emptied and then washed 3-5 times using 300 μl wash solution (0.1 M phosphate-buffered saline or Tris-buffered saline (pH 7.4), 0.02%-0.05% (v/v) Tween 20) per wash. Antibodies at a concentration about 0.1-1.0 μg/ml protein in blocking solution are added to the wells of replica plates, and the plates are incubated at 4° C. overnight or at room temperature for 1 hour to overnight. The plates are again emptied and washed as 3-5 times as before. To detect the antibodies bound to the plates, a secondary antibody solution comprising an antibody that binds to the first antibody (e.g., goat anti-mouse Ig or goat anti-rabbit Ig) conjugated to horseradish peroxidase enzyme (e.g., Alpha Diagnostic International, Inc., San Antonio, Tex. 78238 USA) is employed. The secondary antibody is diluted in 1× blocking solution at a final concentration of about 0.1-1.0 μg/ml protein. About 100 μl diluted secondary antibody is added to each well, and plates are incubated for about 1 hour at room temperature. Plates are again emptied and washed, and reacted with horseradish peroxidase enzyme substrate (KPL, Gaithersburg, Mass. 20879-4174, USA). Reactions are stopped and the absorbances of the wells are read using a plate reader. Wavelengths used will depend on the substrate employed e.g., ABTS (405-410 nm), TMB (non-stopped 620-650 nm, stopped 450 nm), OPD (non-stopped 450 nm, stopped 490 nm), pNPP (405-410 nm), BluePhosÔ (595-650 nm). The signals are compared and the ratios of each bound antibody is determined for each replica sample.

### EXAMPLE 5

### Immunoassays for Determining Cbl Expression

Antibodies

Mouse antibodies against residues 595-810 of human c-Cbl protein are obtained from Clontech. Such antibodies are cross-reactive with c-Cbl proteins from chicken, dog, mouse and rat. A mouse monoclonal antibody that is reactive against residues 695-705 of human c-Cbl protein is supplied by AG Scientific, Inc. A rabbit or goat antibody that binds to the C-terminal region of human c-Cbl and is useful for detecting full-length c-Cbl protein (Santa Cruz Biotechnology, Inc).

Lysate Preparation

Cells are lysed for about 30 min at 40C with buffer containing 50 mM Tris-HCl, pH 8.0, 135 mN NaCl, 1% Triton X-100, 1.0 mM EDTA, 1.0 mM sodium pyrophosphate, 1.0 mM sodium orthovanadate, 10 mM NaF and protease inhibitors (Roche Diagnostics; 1 tablet per 7 ml buffer).

ELISA Assay Format I

Cell lysates are diluted in coating solution (50 mM sodium carbonate, pH 9.6; or 20 mM Tris-HCl, pH 8.5; or 10 mM PBS, pH 7.2) to a concentration of about 0.1-10 μg/ml protein. The diluted antigen is adsorbed onto the wells of 96 or 256 well microtiter plates by adding 50-100 μl antigen in coating solution to the wells and incubating for about 1 hour at room temperature. The plates are emptied and then 300 μl blocking solution (1-10% (w/v) BSA in coating solution, or 1-10% (w/v) nonfat dry milk in coating solution, or 1-10% (w/v) casein in coating solution, or 1-10% (w/v) gelatin in coating solution) is added to the wells. The plates are incubated again at room temperature for about 1 hour. The plates are emptied and then washed 3-5 times using 300 μl wash solution (0.1 M phosphate-buffered saline or Tris-buffered saline (pH 7.4), 0.02%-0.05% (v/v) Tween 20) per wash. Anti-Cbl antibodies at a concentration 0.1-1.0 μg/ml protein in blocking solution are added to the wells of replica plates, and the plates are incubated at 4° C. overnight or at room temperature for 1 hour to overnight. Different anti-Cbl antibodies can be added to each replica plate at this stage, for enhanced accuracy and validation of results. The plates are again emptied and washed as 3-5 times as before. To detect the anti-Cbl antibodies bound to the plates, a secondary antibody solution comprising an antibody that binds to the first antibody (e.g., goat anti-mouse Ig or goat anti-rabbit Ig) conjugated to horseradish peroxidase enzyme (e.g., Alpha Diagnostic International, Inc., San Antonio, Tex. 78238 USA) is employed. The secondary antibody is diluted in 1× blocking solution at a final concentration of about 0.1-1.0 μg/ml protein. About 100 μl diluted secondary antibody is added to each well, and plates are incubated for about 1 hour at room temperature. Plates are again emptied and washed, and reacted with horseradish peroxidase enzyme substrate (KPL, Gaithersburg, Mass. 20879-4174, USA). Reactions are stopped and the absorbances of the wells are read using a plate reader. Wavelengths used will depend on the substrate employed e.g., ABTS (405-410 nm), TMB (non-stopped 620-650 nm, stopped 450 nm), OPD (non-stopped 450 nm, stopped 490 nm), pNPP (405-410 nm), BluePhosÔ (595-650 nm).

ELISA Assay format II (Two-Site ELISA)

A mouse, rabbit or goat anti-human Cbl antibody is diluted in coating solution (50 mM sodium carbonate, pH 9.6; or 20 mM Tris-HCl, pH 8.5; or 10 mM PBS, pH 7.2) to a concentration of about 1-10 μg/ml protein. The diluted antibody is adsorbed onto the wells of 96 or 256 well microtiter plates by adding 50-100 μl antibody in coating solution to the wells and incubating for about 1 hour at room temperature. The plates are emptied and then 300 μl blocking solution (1-10% (w/v) BSA in coating solution, or 1-10% (w/v) nonfat dry milk in coating solution, or 1-10% (w/v) casein in coating solution, or 1-10% (w/v) gelatin in coating solution) is added to the wells. The plates are incubated again at room temperature for about 1 hour. The plates are emptied and then washed 3-5 times using 300 μl wash solution (0.1 M phosphate-buffered saline or Tris-buffered saline (pH 7.4), 0.02%-0.05% (v/v) Tween 20) per wash. Cell lysates are added to the wells of replica plates, optionally comprising 1-10% (w/v) BSA, and the plates are incubated at 4° C. overnight or at room temperature for 1 hour to overnight. The plates are again emptied and washed as 3-5 times as before. A second anti-Cbl antibody that is different from the first antibody used and binds to a different part of the Cbl protein, at a concentration 0.1-1.0 μg/ml protein in 1× blocking solution, is added to the wells and the plates are incubated for about 1 hour at room temperature. Plates are again washed as before.

To detect the second anti-Cbl antibody bound to the plates, a tertiary antibody solution that binds to the secondary antibody (e.g., a goat anti-mouse Ig or goat anti-rabbit Ig) conjugated to horseradish peroxidase enzyme (e.g., Alpha Diagnostic International, Inc., San Antonio, Tex. 78238 USA) is employed. The tertiary antibody is diluted in 1× blocking solution at a final concentration of about 0.1-1.0 μg/ml protein. About 100 μl diluted tertiary antibody is added to each well, and plates are incubated for about 1 hour at room temperature. Plates are again emptied and washed, and reacted with horseradish peroxidase enzyme substrate (KPL, Gaithersburg, Mass. 20879-4174, USA). Reactions are stopped and the absorbances of the wells is read using a plate reader. Wavelengths used will depend on the substrate employed e.g., ABTS (405-410 nm), TMB (non-stopped 620-650 nm, stopped 450 nm), OPD (non-stopped 450 nm, stopped 490 nm), pNPP (405-410 nm), BluePhosÔ (595650 nm).

### EXAMPLE 6

### Assay to Determine Ubiquitin Ligase Activity of Cbl

Antibodies

Anti-ubiquitin antibody is obtained from Santa Cruz Laboratory. Anti-insulin receptor antibody 83-14 is obtained from Abcam, Inc, Cambridge Mass. 02139, USA.

Lysate Preparation and Sandwich ELISA Assay

CHO.T-APS cells are stimulated with insulin at 37° C. for 10-60 mins, and lysates prepared as described by Kotani et al., Biochem J. 335, 103-109, 1998; and Ahmed et al, Biochem J., 341, 665-668, 1991. Briefly, cells are washed in ice-cold phosphate-buffered saline and then lysed for about 30 min at 4° C. with buffer containing 50 mM Tris-HCl, pH 8.0, 135 mN NaCl, 1% Triton X-100, 1.0 mM EDTA, 1.0 mM sodium pyrophosphate, 1.0 mM sodium orthovanadate, 10 mM NaF and protease inhibitors (Roche Diagnostics; 1 tablet per 7 ml buffer).

Anti-insulin receptor antibody 83-14 is diluted in coating solution (50 mM sodium carbonate, pH 9.6; or 20 mM Tris-HCl, pH 8.5; or 10 mM PBS, pH 7.2) to a concentration of about 1-10 μg/ml protein. The diluted antibody is adsorbed onto the wells of 96 or 256 well microtiter plates by adding 50-100 μl antibody in coating solution to the wells and incubating for about 1 hour at room temperature. The plates are emptied and then 300 μl blocking solution (1-10% (w/v) BSA in coating solution, or 1-10% (w/v) nonfat dry milk in coating solution, or 1-10% (w/v) casein in coating solution, or 1-10% (w/v) gelatin in coating solution) is added to the wells. The plates are incubated again at room temperature for about 1 hour. The plates are emptied and then washed 3-5 times using 300 μl wash solution (0.1 M phosphate-buffered saline or Tris-buffered saline (pH 7.4), 0.02%-0.05% (v/v) Tween 20) per wash. Cell lysates are added to the wells of replica plates, optionally comprising 1-10% (w/v) BSA, and the plates are incubated at 4° C. overnight or at room temperature for 1 hour to overnight. The plates are again emptied and washed as 3-5 times as before. Anti-ubiquitin antibody at a concentration 0.1-1.0 μg/ml protein in 1× blocking solution is added to the wells and the plates are incubated for about 1 hour at room temperature. Plates are again washed as before.

To detect the anti-ubiquitin antibody bound to the plates, a tertiary antibody solution comprising goat anti-mouse Ig conjugated to horseradish peroxidase enzyme (e.g., Alpha Diagnostic International, Inc., San Antonio, Tex. 78238 USA) is employed. The tertiary antibody is diluted in 1× blocking solution at a final concentration of about 0.1-1.0 μg/ml protein. About 100 μl diluted tertiary antibody is added to each well, and plates are incubated for about 1 hour at room temperature. Plates are again emptied and washed, and reacted with horseradish peroxidase enzyme substrate (KPL, Gaithersburg, Mass. 20879-4174, USA). Reactions are stopped and the absorbances of each well is determined using a plate reader. Wavelengths used will depend on the substrate employed e.g., ABTS (405-410 nm), TMB (non-stopped 620-650 nm, stopped 450 nm), OPD (non-stopped 450 nm, stopped 490 nm), pNPP (405-410 nm), BluePhosÔ (595-650 nm).

### EXAMPLE 7

### Production and Use of CHO.T-APS Cells Expressing Human Cbl

Expression Vectors and Cell Lines

CHO.T-APS Cells

CHO.T cells (Ebina et al., Proc. Natl Acad. Sci USA 82, 8014-8018, 1985) stably-expressing a Myc-tagged rat APS from the vector pIRES Hygro (Clontech) are produced as described by Ahmed et al., FEBS Letts 475, 31-34, 2000. Briefly, the Myc-tagged APS (Qian et al., Neuron 21, 1017-1029, 1998) is subcloned into pIRES Hygro and transfected into CHO.T cells using Superfect (Qiagen), and transfected cells are selected in media comprising about 800 μg/ml hygromycin. Clones are purified by limiting dilution and screened using anti-myc 9E10 monoclonal antibody prepared from a publicly available 9E10 hybridoma. Clones are maintained in media comprising about 400 μg/ml hygromycin.

CHO.T-APS Cells Expressing Human c-Cbl and Dominant Negative c-Cbl Mutants

Several dominant-negative mutants of human c-Cbl are described in the literature, including c-Cbl G306E (SEQ ID NO: 250), c-Cbl C381A (SEQ ID NO: 252), c-Cbl Y700F (SEQ ID NO: 254), c-Cbl Y731F (SEQ ID NO: 256), c-Cbl Y774F (SEQ ID NO: 258) and c-Cbl 480 (SEQ ID NO: 262). These known mutants provide useful negative controls to assess the ability of a putative inhibitor of c-Cbl-mediated ubiquitination of IR-β in isolated cells. Accordingly, the open reading frames encoding these dominant negative mutants (SEQ ID Nos: 249, 251, 253, 255, 257 and 261, respectively) are separately sub-cloned into the retroviral expression vector pBabe-Puro retroviral expression vector (Morgenstem and Land, Nucleic Acids Res. 18, 3587-3596, 1990), to produce the control vectors pBabe-Cbl G306E, pBabe-C381A pBabe-Cbl Y700F, pBabe-Cbl Y731F, pBabe-Y774F and pBabe-Cbl 480.

A further control plasmid comprising the full-length human c-Cbl open reading frame is also produced in the pBabe vector.

A test plasmid is also produced that are capable of expressing a further putative dominant negative mutant of human c-Cbl, designated c-Cbl Y700F/Y731F/Y774F (SEQ ID NO: 260). This mutant has mutations in the open reading frame of c-Cbl that result in three tyrosine residues that would normally be phosphorylated being substituted for phenylalanine. The nucleotide sequence of the open reading frame encoding c-Cbl Y700F/Y731F/Y774F is set forth in SEQ ID NO: 259. The mutant is produced using the Stratagene Quick Change mutagenesis kit according to the manufacturer's instructions. Confirmation of the correct mutations is obtained by standard sequence analysis. The open reading frame encoding c-Cbl Y700F/Y731F/Y774F is cloned into pBabe-Puro, to produce the test vector pBabe-Cbl Y700F/Y731F/Y774F.

BOSC23 packaging cells (Pear et al., Proc. Natl. Acad. Sci. USA, 90, 8392-8396, 1993) are maintained in DMEM containing 10% FBS at 37° C. in a humidified atmosphere of 10% CO₂. Cells are then transfected by calcium phosphate co-precipitation with 10 μg each of the plasmids.

Transiently produced viral supernatants are used to infect CHO.T-APS cells in the presence of 4 μg/ml Polybrene. After elimination of uninfected cells by puromycin, stable cell lines are maintained in DMEM containing 10% FBS.

Alternatively, CHO.T-APS cells are transfected using Superfect (Qiagen), and transfected cells are selected, purified by limiting dilution and maintained in media comprising puromycin and 400 μg/ml hygromycin.

CHO.T-APS Cells Expressing Human c-Cbl and Transfected with siRNAs and shRNAs Targeting Human c-Cbl Expression

The native human c-Cbl gene sequence (SEQ ID NO: 263; GenBank Accession No. X57110) was analyzed using the siRNA Target Finder software available from Ambion, Inc. (Austin, Tex., USA) to determine those sequences most likely to silence c-Cbl expression when used to produce siRNA and/or shRNA (Table 4; SEQ ID NOs: 4-241). Further sequence analysis using the BLAST programme indicated that several of these sequences were not present in the murine c-Cbl gene, in particular SEQ ID NOS: SEQ ID Nos: 59-61, 119-123, 177-179 and 237-241.

Oligonucleotides molecules are chemically synthesised that consist of the nucleotide sequences set forth in Table 4. The sense and antisense stands of the siRNA molecules are annealed and introduced directly into CHO.T-APS cells expressing human c-Cbl or dominant negative mutated forms thereof. Briefly, CHO.T-APS cells are seeded onto 96 well tissue culture plates approximately 24 hr before transfection and grown to approximately 30-70% confluence. Prior to transfection, single stranded siRNA molecules are diluted in OPTI-MEM I reduced serum medium (Ambion) to a final concentration of approximately 0.5 μM. siRNA solutions are then added to siPORT Lipid (Ambion) that has been previously diluted in OPTI-MEM I reduced serum medium. This final solution is then incubated at room temperature for approximately 20 minutes.

The CHO.T-APS cells are then washed with OPTI-MEM I reduced serum medium and overlayed with fresh OPTI-MEM I reduced serum medium. The transfection agent/siRNA complex is then added to each well of the plate, and plates incubated for 4 hours at 37° C. supplemented with 5% CO₂. Following 4 hours additional DMEM (supplemented with 10% FBS) is added to each well, and cells cultured at 37° C. supplemented with 5% CO₂.

For construction of shRNA, a loop sequence (TTCAAGAGA, SEQ ID NO: 248) is positioned between complementary sense and antisense strands (SEQ ID NOs: 6-241) to facilitate hairpin formation. Oligonucleotides that are complementary to the assembled shRNA are also produced synthetically. Both strands of each shRNA molecule are then mixed in equimolar amounts, heated to 95° C. for 10 minutes and allowed to cool. Double stranded shRNA-encoding DNAs are cloned into the pAdTrack-HP expression vector (Zhao et al., Gene 316: 137-141, 2003), upstream of the Pol III transcriptional termination sequence. The pAdTrack-HP expression vector comprises BglII and HindIII recognition sequences to facilitate directional cloning of shRNA upstream of the Pol III transcriptional terminator.

Control plasmids comprising the full-length human c-Cbl open reading frame in the sense and antisense orientations are also produced in the pAdTrack-HP expression vector.

Adenoviruses comprising the shRNA constructs are produced essentially as described by He et al., Proc. Natl. Acad. Sci. USA. 95: 2509-2514, 1998. Briefly, the control vectors and the library of pAdTrack-shRNA vectors are linearized with PmeI and transfected into AdEasier-1 cells or E. coli BJ5183 cells carrying the pAdEasy-1 plasmid. The pAdEasy-1 adenoviral plasmid contains all Ad5 sequences except nucleotides 1-3,533 (encompassing the E1 genes) and nucleotides 28,130-30,820 (encompassing E3). Recombinant viral genomes are linearized with PacI and transfected into 293 cells in a six-well plate using lipofectamine 2000 (Invitrogen). Eight days after transfection, the recombinant virus is collected and subjected to one round of amplification in a T-25 flask with 1.5×10⁶ 293 cells, resulting in 2 ml of viral stocks. CHO.T-APS cells in a 96-well plate are infected with Adenovirus comprising the pADTrack-shRNA library for 2 h, washed, and incubated with medium for 4 days.

TABLE 4

Human c-CbI siRNA oligonucleotides for silencing
human c-CbI gene expression

| | | | |
|---|---|---|---|
| Sense strand siRNA | SEQ ID NO: | Antisense strand siRNA | SEQ ID NO: |

| | | | |
|---|---|---|---|
| CGTGAAGAAGAGCTCTGGGTT | 6 | CCCAGAGCTCTTCTTCACGTT | 124 |
| GAAGATGGTGGAGAAGTGCTT | 7 | GCACTTCTCCACCATCTTCTT | 125 |
| GATGGTGGAGAAGTGCTGGTT | 8 | CCAGCACTTCTCCACCATCTT | 126 |
| GTGCTGGAAGCTCATGGACTT | 9 | GTCCATGAGCTTCCAGCACTT | 127 |
| GCTCATGGACAAGGTGGTGTT | 10 | CACCACCTTGTCCATGAGCTT | 128 |
| GGTGGTGCGGTTGTGTCAGTT | 11 | CTGACACAACCGCACCACCTT | 129 |
| CCCAAAGCTGGCGCTAAAGTT | 12 | CTTTAGCGCCAGCTTTGGGTT | 130 |
| CCCAAAGCTGGCGCTAAAGTT | 13 | CTTTAGCGCCAGCTTTGGGTT | 131 |
| AGCTGGCGCTAAAGAATAGTT | 14 | CTATTCTTTAGCGCCAGCTTT | 132 |
| AGAATAGCCCACCTTATATTT | 15 | ATATAAGGTGGGCTATTCTTT | 133 |
| TAGCCCACCTTATATCTTATT | 16 | TAAGATATAAGGTGGGCTATT | 134 |
| GATATGAGGGGAAGATGGATT | 17 | TCCATCTTCCCCTCATATCTT | 135 |
| GATGGAGACACTTGGAGAATT | 18 | TTCTCCAAGTGTTTCCATCTT | 136 |
| CTAAGCAAACCATAAGCCTTT | 19 | AGGCTTATGGTTTGTTTAGTT | 137 |
| GCAAACCATAAGCCTCTTCTT | 20 | GAAGAGGCTTATGGTTTGCTT | 138 |
| ACCATAAGCCTCTTCAAGGTT | 21 | CCTTGAAGAGGCTTATGGTTT | 139 |
| GCCTCTTCAAGGAGGGAAATT | 22 | TTTCCCTCCTTGAAGAGGCTT | 140 |
| GAAAGAATGTATGAGGAGATT | 23 | TCTCCTCATACATTCTTTCTT | 141 |
| AGAATGTATGAGGAGAATTTT | 24 | AATTCTCCTCATACATTCTTT | 142 |
| TGTATGAGGAGAATTCTCATT | 25 | TGAGAATTCTCCTCATACATT | 143 |
| TTCTCAGCCTAGGCGAAACTT | 26 | GTTTCGCCTAGGCTGAGAATT | 144 |
| ACCTAACCAAACTGTCCCTTT | 27 | AGGGACAGTTTGGTTAGGTTT | 145 |
| CCAAACTGTCCCTCATCTTTT | 28 | AAGATGAGGGACAGTTTGGTT | 146 |
| ACTGTCCCTCATCTTCAGCTT | 29 | GCTGAAGATGAGGGACAGTTT | 147 |
| GGAATC1TTCCAAGTGGACTT | 30 | GTCCACTTGGAAAGATTCCTT | 148 |
| TCTTTCCAAGTGGACTCTTTT | 31 | AAGAGTCCACTTGGAAAGATT | 149 |
| GTGGACTCTTTCAGGGAGATT | 32 | TCTCCCTGAAAGAGTCCACTT | 150 |
| AGCAGATGCTGCGGAATTTTT | 33 | AAATTCCGCAGCATCTGCTTT | 151 |
| GACAATAGTCCCTTGGAAGTT | 34 | CTTCCAAGGGACTATTGTCTT | 152 |
| TAGTCCCTTGGAAGAGCTTTT | 35 | AAGCTCTTCCAAGGGACTATT | 153 |
| GAGCTTTCGACAGGCTCTATT | 36 | TAGAGCCTGTCGAAAGCTCTT | 154 |
| GTGCATCCCATCAGTTCTGTT | 37 | CAGAACTGATGGGATGCACTT | 155 |
| ATCCACTATTGATCTGACCTT | 38 | GGTCAGATCAATAGTGGATTT | 156 |
| TTTGACATCTTTACCCGACTT | 39 | GTCGGGTAAAGATGTCAAATT | 157 |
| TTGGAACAGCCTTGCTGTATT | 40 | TACAGCAAGGCTGTTCCAATT | 158 |
| CAGCCTTGCTGTAACTCATTT | 41 | ATGAGTTACAGCAAGGCTGTT | 159 |
| CTCATCCTGGCTACATGGCTT | 42 | GCCATGTAGCCAGGATGAGTT | 160 |
| GTGAAAGCTCGGCTCCAGATT | 43 | TCTGGAGCCGAGCTTTCACTT | 161 |
| AGCTCGGCTCCAGAAATTCTT | 44 | GAATTTCTGGAGCCGAGCTTT | 162 |
| ATTCATTCACAAACCTGGCTT | 45 | GCCAGGTTTGTGAATGAATTT | 1131 |
| ACCTGGCAGTTATATCTTCTT | 46 | GAAGATATAACTGCCAGGTTT | 164 |
| CATTCTCCAGACAATCCCTTT | 47 | AGGGATTGTCTGGAGAATGTT | 165 |
| TCCCTCACAATAAACCTCTTT | 48 | AGAGGTTTATTGTGAGGGATT | 166 |
| TAAACCTCTCTTCCAAGCATT | 49 | TGCTTGGAAGAGAGGTTTATT | 167 |
| ACCTCTCTTCCAAGCACTGTT | 50 | CAGTGCTTGGAAGAGAGGTTT | 168 |
| GCACTGATTGATGQCTTCATT | 51 | TGAAGCCATCAATCAGTGCTT | 169 |
| GGCTTCTATTTGTTTCCTGTT | 52 | CAGGAAACAAATAGAAGCCTT | 170 |
| ATCAGAATCCTGATCTGACTT | 53 | GTCAGATCAGGATTCTGATTT | 171 |
| TCCTGATCTGACTGGCTTATT | 54 | TAAGCCAGTCAGATCAGGATT | 172 |
| CCAACTCCCCAAGACCATATT | 55 | TATGGTCTTGGGGAGTTGGTT | 173 |
| CTCCCCAAGACCATATCAATT | 56 | TTGATATGGTCTTGGGGAGTT | 174 |
| GACCATATCAAAGTGACCCTT | 57 | GGGTCACTTVGATATGGTCTT | 175 |
| AGTGACCCAGGAACAATATTT | 58 | ATATTGTTCCTGGGTCACTTT | 176 |
| CAATATGAATTATACTGTGTT | 59 | CACAGTATAATTCATATTGTT | 177 |
| TATGAATTATACTGTGAGATT | 60 | TCTCACAGTATAATTCATATT | 178 |
| TTATACTGTGAGATGGGCTTT | 61 | AGCCCATCTCACAGTATAATT | 179 |
| TGATAAGGATGTAAAGATTTT | 62 | AATCTTTACATCCTTATCATT | 180 |
| GGATGTAAAGATTGAGCCCTT | 63 | GGGCTCAATCTTTACATCCTT | 181 |
| AGATTGAGCCCTGTGGACATT | 64 | TGTCCACAGGGCTCAATCTTT | 182 |
| TCAGAAGGTCAGGGCTGTCTT | 65 | GACAGCCCTGACCTTCTGATT | 183 |
| GGTCAGGGCTGTCCTTTCTTT | 66 | AGAAAGGACAGCCCTGACCTT | 184 |
| ATTAAAGGTACTGAACCCATT | 67 | TGGGTTCAGTACCTTTAATTT | 185 |
| AGGTACTGAACCCATCGTGTT | 68 | CACGATGGGTTCAGTACCTTT | 186 |
| CCCATCGTGGTAGATCCGTTT | 69 | ACGGATCTACCACGATGGGTT | 187 |
| ATTATGATGATGATGATGATT | 70 | TCATCATCATCATCATAATTT | 188 |
| CGAGCTGATGATACTCTCTTT | 71 | AGAGAGTATCATCAGCTCGTT | 189 |
| GGAATTGGCTGGTGCCAAGTT | 72 | CTTGGCACCAGCCAATTCCTT | 190 |
| TTGGCTGGTGCCAAGGTGGTT | 73 | CCACCTTGGCACCAGCCAATT | 191 |
| CGGCCGCCTTCTCCATTCTTT | 74 | AGAATGGAGAAGGCGGCCGTT | 192 |
| GTGCTTCTGCTCTTGGAACTT | 75 | GTTCCAAGAGCAGAAGCACTT | 193 |
| CTGCTTCTAAGGCTGCTTCTT | 76 | GAAGCAGCCTTAGAAGCAGTT | 194 |
| GGCTGCTTCTGGCTCCCTTTT | 77 | AAGGGAGCCAGAAGCAGCCTT | 195 |
| AGACAAACCATTGCCAGTATT | 78 | TACTGGCAATGGTTTGTCTTT | 196 |
| ACCATTGCCAGTACCTCCCTT | 79 | GGGAGGTACTGGCAATGGTTT | 197 |
| TCCCGACCTCAAAGACGCCTT | 80 | GGCGTCTTTGAGGTCGGGATT | 198 |
| AGACGCCCCTTGCCTTGTATT | 81 | TACAAGGCAAGGGGCGTCTTT | 199 |
| TCCCCAAAGTACCAGTATCTT | 82 | GATACTGGTACTTTGGGGATT | 200 |
| AGTACCAGTATCTGCCCCATT | 83 | TGGGGCAGATACTGGTACTTT | 201 |
| GTTCCAGTGATCCCTGGACTT | 84 | GTCCAGGGATCACTGGAACTT | 202 |
| GAGAATTAACCAACCGGCATT | 85 | TGCCGGTTGGTTAATTCTCTT | 203 |
| TTAACCAACCGGCACTCACTT | 86 | GTGAGTGCCGGTTGGTTAATT | 204 |
| CCAACCGGCACTCACTTCCTT | 87 | GGAAGTGAGTGCCGGTTGGTT | 205 |
| CCGGCACTCACTTCCATTTTT | 88 | AAATGGAAGTGAGTGCCGGTT | 206 |
| ATGGAGCCCAGACCAGATGTT | 89 | CATCTGGTCTGGGCTCCATTT | 207 |
| GCACGTTCAGTCTGGATACTT | 90 | GTATCCAGACTGAACGTGCTT | 208 |
| TAGCAGCCCATTAGTAGGTTT | 91 | ACCTACTAATGGGCTGCTAT | 209 |
| TCAAACCTTCCTCATCTGCTT | 92 | GCAGATGAGGAAGGTTTGATT | 210 |
| ACCTTCCTCATCTGCCAATTT | 93 | ATTGGCAGATGAGGAAGGTTT | 211 |
| TGCCATTTATTCTCTGGCTTT | 94 | AGCCAGAGAATAAATGGCATT | 212 |
| CTGCCACCTGGGGAGCAATTT | 95 | ATTGCTCCCCAGGTGGCAGTT | 213 |
| TGTGAGGGTGAAGAGGACATT | 96 | TGTCCTCTTCACCCTCACATT | 214 |
| GAGGACACAGAGTACATGATT | 97 | TCATGTACTCTGTGTCCTCTT | 215 |
| GCAATGTATAATATTCAGTTT | 98 | ACTGAATATTATACATTGGTT | 216 |
| TGTATAATATVCAGTCCCATT | 99 | TGGGACTGPATATTATACATT | 217 |
| TATTCAGTCCCAGGCGCCATT | 100 | TGGCGCCTGGGACTGAATATT | 218 |
| CACTGGTCCCGAGGAGTCATT | 101 | TGACTCCTCGGGACCAGTGTT | 219 |
| TGAGGATGATGGGTATGATTT | 102 | ATCATACCCATCATCCTCATT | 220 |
| CTCTCTCAGATATCTCTAATT | 103 | TTAGAGATATCTGAGAGAGTT | 221 |
| TGCCAGCTCCTCCTTTGGCTT | 104 | GCCAAAGGAGGAGCTGGCATT | 222 |
| CAAATGTCACTGAAGGTTCTT | 105 | GAACCTTCAGTGACATTTGTT | 223 |
| ATGTCACTGAAGGTTCCCATT | 106 | TGGGAACCTTCAGTGACATTT | 224 |
| GGTTCCCAAGTTCCCGAGATT | 107 | TCTCGGGAACTTGGGAACCTT | 225 |
| GTTCCCGAGAGGCCTCCAATT | 108 | TTGGAGGCCTCTCGGGAACTT | 226 |
| CCATTCCCGCGGAGAATCATT | 109 | TGATTCTCCGCGGGAATGGTT | 227 |
| TCAACTCTGAACGGAAAGCT | 110 | GCTTTCCGTTCAGAGTTGATT | 228 |
| CTCTGAACGGAAAGCTGGCTT | 111 | GCCAGCTTTCCGTTCAGAGTT | 229 |
| CGGAAAGCTGGCAGCTGTCTT | 112 | GACAGCTGCCAGCTTTCCGTT | 230 |
| AGCTGGCAGCTGTCAGCAATT | 113 | TTGCTGACAGCTGCCAGCTTT | 231 |
| CCTCATGAGTCAGGGGTACTT | 114 | GTACCCCTGACTCATGAGGTT | 232 |
| AGCTTTGGTCATTGCCCAGTT | 115 | CTGGGCAATGACCAAAGCTTT | 233 |
| CAACATCGAGATGGCCAAATT | 116 | TTTGGCCATCTCGATGTTGTT | 234 |
| ACATCCTCCGGGAATTTGTTT | 117 | ACAAATTCCCGGAGGATGTTT | 235 |
| TTTGTTTCCATTTCTTCTCTT | 118 | GAGAAGAAATGGAAACAAATT | 236 |
| GTGGCACCTAGAAGGGCAGTT | 119 | CTGCCCTTCTAGGTGCCACTT | 237 |
| GGGCAGGAGTTCCTTTGGTTT | 120 | ACCAAAGGAACTCCTGCCCTT | 238 |
| GTCTTGCCCTCTCTGTGGGTT | 121 | CCCACAGAGAGGGCAAGACTT | 239 |
| GATTTCAAAGTGGTGAAATTT | 122 | ATTTCACCACTTTGAAATCTT | 240 |
| TGGAGCAGCTAGTATGTTTTT | 123 | AAACATACTAGCTGCTCCATT | 241 |

### EXAMPLE 6

### Production and use of C2C12 Cells Expressing Human Cbl

Expression Vectors and Cell Lines

C2C12 Cells

C2C12 cells (ATCC CRL-1772; Yaffe et al., Nature 270, 725-727, 1977; Yaffe et al., Differentiation 7, 159-166, 1977) are a murine myoblast cell line that differentiates rapidly to form contractile myotubes and to produce characteristic muscle proteins. Cultures are maintained as non-confluent cultures to prevent depletion of myoblasts. Myotube formation is enhanced when cells are grown in medium supplemented with 10% (v/v) horse serum instead of fetal bovine serum.

C2C12 Cells Expressing Human c-Cbl and Dominant Negative c-Cbl Mutants

The dominant-negative mutants of human c-Cbl designated c-Cbl G306E (SEQ ID NO: 250), c-Cbl C381A (SEQ ID NO: 252), c-Cbl Y700F (SEQ ID NO: 254), c-Cbl Y731F (SEQ ID NO: 256), and c-Cbl Y774F (SEQ ID NO: 258) expressed from the retroviral expression vector pBabe-Puro (Morgenstern and Land, Nucleic Acids Res. 18, 3587-3596, 1990), to produce the control vectors pBabe-Cbl G306E, pBabe-C381A pBabe-Cbl Y700F, pBabe-Cbl Y731F, and pBabe-Y774F, essentially as described in the preceding example.

A further control plasmid comprising the full-length human c-Cbl open reading frame is also produced in the pBabe vector.

A test plasmid is also produced that is capable of expressing a further putative dominant negative mutant of human c-Cbl, designated c-Cbl Y700F/Y731F/Y774F (SEQ ID NO: 260), as described in the preceding example.

BOSC23 packaging cells (Pear et al., Proc. Natl. Acad. Sci. USA, 90, 8392-8396, 1993) are maintained in DMEM containing 10% FBS at 37° C. in a humidified atmosphere of 10% CO₂. Cells are then transfected by calcium phosphate co-precipitation with 10 μg each of the plasmids supra.

Transiently produced viral supernatants are used to infect C2C12 cells expressing human c-Cbl or dominant negative mutant forms thereof, in the presence of 4 μg/ml Polybrene. After elimination of uninfected cells by puromycin, stable cell lines are maintained in DMEM containing 10% FBS.

Alternatively, C2C12 cells are transfected using Superfect (Qiagen), and transfected cells are selected, purified by limiting dilution and maintained in media comprising puromycin and 400 μg/ml hygromycin.

Humanized C2C12 Myoblasts Expressing siRNAs and shRNAs Targeting Human c-Cbl Expression

SiRNAs and shRNAs and control vectors are produced as described in the preceding example. The siRNA oligonucleotides are introduced directly into C2C12 cells expressing human c-Cbl or dominant negative mutant forms thereof by seeding the cells onto 96 well tissue culture plates approximately 24 hr before transfection, growing the cells approximately 30-70% confluence (it is preferable to ensure that confluence is not reached to prevent myoblast depletion) and immediately prior to transfection, diluting single stranded siRNA molecules in OPTI-MEM I reduced serum medium (Ambion) to a final concentration of approximately 0.5 μM, adding the siRNA solutions to siPORT Lipid (Ambion) that has been previously diluted in OPTI-MEM I reduced serum medium and incubating the reaction mixture at room temperature for approximately 20 minutes.

The humanized C2C12 cells are then washed with OPTI-MEM I reduced serum medium and overlayed with fresh OPTI-MEM I reduced serum medium. The transfection agent/siRNA complex is then added to each well of the plate, and plates incubated for 4 hours at 37° C. supplemented with 5% CO₂. Following 4 hours additional DMEM (supplemented with 10% FBS) is added to each well, and cells cultured at 37° C. supplemented with 5% CO₂.

For stable introduction of shRNAs into Cbl-humanized C2C12 cells, Adenovirus comprising the pADTrack-shRNA library produced as described in the preceding example is used to infect the cells in a 96-well plate for 2 h, after which time the cells are washed and incubated with medium for 4 days.

Cell lysates derived from non-transfected C2C12 cells transfected with control vectors expressing human c-Cbl in the sense orientation will have detectable levels of human c-Cbl, as determined by ELISA assay formats I or II. In marked contrast, cell lysates derived from cells comprising the dominant-negative mutant constructs pBabe-Cbl G306E, pBabe-C381A, pBabe-Cbl Y700F, pBabe-Cbl Y731F, or pBabe-Y774F will bind reduced or non-detectable levels of anti-Cbl antibody in assay format 1, or following antibody-capture of Cbl and subsequent two-site ELISA according to assay format II.

Such data suggest that the effects of inhibitors of c-Cbl expression can be assayed-directly by measuring c-Cbl protein levels in cell lysates from transfected C2C12 cells, in standard ELISA assay formats. Other immunoassay formats are also contemplated to be applicable in this respect.

Proceeding on this basis, c-Cbl protein levels are assayed in C2C12 cells expressing a c-Cbl antisense RNA or the dominant negative mutant c-Cbl Y700F/Y731F/Y774F using the sandwich ELISA assays herein. Reduced levels of c-Cbl protein detectable in C2C12-cells expressing these molecules, relative to the level of ubiquitin bound to the receptor in lysates from cells expressing the full-length c-Cbl open reading frame in the sense orientation, indicate that these molecules are also effective antagonists of c-Cbl expression.

Additionally, an Adenovirus (Ad5) library that expresses a series of pADTrack-shRNAs designed against human c-Cbl expression (Table 4) is shotgun-cloned into C2C12 cells, and the level of c-Cbl protein is determined. A control plasmid comprising the full-length open reading frame of wild-type human c-Cbl is also introduced. Empty vector controls and non-transfected controls are also employed. Those cells that produce lysates having reduced levels of c-Cbl protein as determined by ELISA assay format I and/or ELISA assay format II are retained for further analysis, such as for introduction into animal models for validation. Alternatively, or in addition, the corresponding shRNAs are introduced into C2C12 cells in the pBabe vector for assessment of their effects on muscle thermogenesis.

### EXAMPLE 7

### Screening Assays Performed in Animal Models

Production of a Human c-Cbl Knock-In Mouse Model

A 5-Cbl strain 129 mouse genomic fragment containing sequences upstream of the mouse c-Cbl gene (about 2-3 kbp) and including exon 1 and the 5′ part of intron 1 is generated by PCR from mouse chromosomal DNA or a chromosome 9 BAC comprising the c-Cbl gene and flanking sequences. A 3-Cbl strain 129 mouse genomic fragment, containing the 3′ half of the final exon and 3-flanking sequences of the mouse c-Cbl gene (about 2-3 kbp) is also derived by PCR.

Human genomic fragments, containing the 3′ part of intron 1 through to and including the final exon of the human c-Cbl gene are isolated by sequential digestion from plasmids.

To obtain gene targeting constructs, the 5-Cbl mouse genomic fragment and the human genomic fragments are inserted into a pPN2T vector (Paszty et al., Nat. Genet. 11, 33-39, 1995) upstream of the neomycin-resistant gene. The mouse 3-Cbl genomic fragment is inserted downstream of the neomycin-resistant gene. The arrangement of the inserts in the targeting vector are such that the 5-Cbl and 3-Cbl arms of mouse homology are interrupted by the human Cbl gene and the neomycin-resistant gene, and this cassette is upstream of the thymidine kinase gene in the vector. The targeting vector is linearized, purified, and redissolved in TE (10 mM Tris-HCl, pH 8, 1 mM EDTA), for electroporation.

A subclone of mouse strain 129 embryonic stem cell line, ESVJ (Go Germline, GenomeSystems, Inc.), is cultured on neomycin-resistant mouse fibroblast feeder layers and electroporated with 20 μg of the linearized targeting vector essentially as described by Koller, et al., Proc. Natl. Acad. Sci. USA 88, 10730-10734, 1991. Stable integrants are selected by positive-negative selection, using neomycin (G418-Geneticin; Invitrogen) at a final concentration of 200 μg/ml and 2 μM gancyclovir (FIAU, Moravek Biochemicals, Brea, Calif.). After 10-12 days, the colonies are transferred into 96-well plates and tested for successful targeting and lack of rearrangement of the introduced human Cbl gene, by Southern blotting using conventional procedures.

Approximately 10-15 embryonic stem-targeted cells are injected into the blastocele cavity of C57BL/6J embryos. Surviving blastocysts are transferred into the pseudopregnant CD-1 females. Animals chimeric by coat color are bred to C57BL/6J animals to determine their germ line competency. Heterozygous mutants are identified by Southern blotting of DNA isolated from the tail, and brother-sister mating is carried out to generate homozygous knock-in mutant mouse lines expressing human c-Cbl.

Expression of Dominant Negative Cbl Mutants in Cb^{+/+} Mouse and Human c-Cbl Knock-In Mouse

Because of the variation in adenoviral delivery to hindlimb muscles, it is preferred to employ an expression vector that permits quantitation of reporter gene expression e.g., the pAdTrack-CMV vector. Accordingly, the open reading frames encoding the dominant-negative mutants of human c-Cbl designated c-Cbl G306E (SEQ ID NO: 250), c-Cbl C381A (SEQ ID NO: 252), c-Cbl Y700F (SEQ ID NO: 254), c-Cbl Y731F (SEQ ID NO: 256), c-Cbl Y774F (SEQ ID NO: 258), c-Cbl 480 (SEQ ID NO: 262) and c-Cbl Y700F/Y731F/Y774F (SEQ ID NO: 260) as described in Example 5, are separately sub-cloned into the shuttle vector pAdTrack-CMV to permit their expression as a GFP fusion protein under the control of the CMV promoter. The GFP tag is not important to function of the Cbl protein moiety. The resultant plasmids are designated pAdTrack-CMV-G306E-GFP, pAdTrack-CMV-C381A-GFP, pAdTrack-CMV-Y700F-GFP, pAdTrack-CMV-Y731F-GFP, pAdTrack-CMV-Y774F-GFP, pAdTrack-CMV-Cbl 480-GFP, and pAdTrack-CMV-Y700F/Y731F/Y774F-GFP.

A further control plasmid comprising the full-length human c-Cbl open reading frame is also produced in the pAdTrack-CMV vector. This plasmid is designated pAdTrack-CMV-Cbl-GFP.

Adenoviruses comprising these constructs are produced essentially as described by He et al., Proc. Natl. Acad. Sci. USA. 95: 2509-2514, 1998. Briefly, the pAdTrack-CMV-based vectors are linearized with PmeI and transfected into AdEasier-1 cells or E. coli BJ5183 cells carrying the pAdEasy-1 plasmid. The pAdEasy-1 adenoviral plasmid contains all Ad5 sequences except nucleotides 1-3,533 (encompassing the E1 genes) and nucleotides 28,130-30,820 (encompassing E3). Recombinant viral genomes are linearized with PacI and transfected into adenoviral packaging 293 cells in a six-well plate using lipofectamine 2000 (Invitrogen). Eight days after transfection, the recombinant virus is collected and subjected to one round of amplification in a T-25 flask with 15×10⁶ 293 cells, resulting in 2 ml of viral stocks.

Adenoviruses expressing the dominant negative c-Cbl mutants are introduced into both wild type mice and the c-Cbl knock-in mouse supra, essentially as described by Allamand et al., Gene Ther. 7, 1385-1391, 2000. This is a robust system for the high efficiency (70-100%) transfer and expression of nucleic acid in mouse hindlimb skeletal muscle, wherein expression is maintained for >8 wk post infection, albeit being restricted to the muscle injected with the adenovirus. Briefly, mice (24 day old) are anesthetised via hypothermia and infectious adenovirus (2×10⁹) containing shRNAs is injected in saline percutaneously into several muscles within the hindlimb. Pups are reintroduced to mothers and studied at 4-8 weeks of age. Relevant muscles are excised at different times after injection (1, 2, 4 and 8 weeks).

Expression of siRNAs and shRNAS in Cbl^{+/+} Mouse and Human c-Cbl Knock-In Mouse

Adenoviruses expressing the siRNAs and shRNAs as described supra are introduced into both wild type mice and the c-Cbl knock-in mouse supra, and mice are treated and analysed, essentially as described in the preceding paragraph.

Effect of Dominant Negative Cbl Mutants, siRNAs and shRNAS on Phenotypes of Cbl^{-/-} Mouse and Human c-Cbl Knock-In Mouse

The ability of inhibitory nucleic acids to mimic the phenotype of the Cbl^{-/-} mouse model is determined by their effect when administered to wild type (Cbl^{+/+} mice) and/or to mice in which the murine Cbl gene has been replaced with a human Cbl-encoding gene (i.e., the Cbl knock-in mouse). The latter murine model is particularly desirable for predicting effects on human c-Cbl in humans. Cbl activity and expression, and mitochondrial ACC and AMPK activities, rates of fatty acid oxidation and levels of free fatty acids are determined for mice from one week of age.

1. A method of identifying a compound that reduces whole body insulin sensitivity of an animal, said method comprising: (a) administering a compound to a non-human animal, isolated tissue or isolated cell having reduced expression of functional Cbl compared to an otherwise isogenic wild-type animal, tissue or cell; and (b) determining a Cbl-associated phenotype selected from the group consisting of: (i) the activity of acetyl CoA carboxylase (ACC) and/or amount of phosphorylated ACC enzyme; (ii) the activity of AMP-dependent protein kinase (AMPK) and/or amount of phosphorylated AMPK enzyme; (iii) expression of mitochondrial uncoupling protein 3 (UCP3): (iv) free fatty acid level and/or fatty acid oxidation; (v) fat content; (vi) lean muscle mass; (vii) muscle thermogenesis; (viii) feeding behavior; (ix) the level of an insulin receptor (IR); (x) whole body energy expenditure; (xi) metabolic rate; and (xii) glucose tolerance in the animal, tissue or cell wherein a reduced amount of phosphorylated ACC enzyme, AMPK enzyme, amount of phosphorylated AMPK enzyme, expression of UCP3, fatty acid oxidation, lean muscle mass, muscle thermogenesis, feeding behaviour, IR level, whole body energy expenditure, metabolic rate or glucose tolerance, and/or enhanced activity of an ACC enzyme, free fatty acid level or fat content compared to the amount of phosphorylated ACC enzyme or activity of an ACC enzyme of a Cbl-deficient animal, tissue or cell to which the compound has not been administered indicates that the compound reduces whole body insulin sensitivity in an animal.

2-23. (canceled)

24. A method of identifying a compound that enhances whole body insulin sensitivity of an animal comprising: (a) administering a compound to a non-human animal, isolated tissue or isolated cell expressing a functional Cbl protein and determining a Cbl-associated phenotype selected from the group consisting of: (i) the activity of acetyl CoA carboxylase (ACC) and/or amount of phosphorylated ACC enzyme; (ii) the activity of AMP-dependent protein kinase (AMPK) and/or amount of phosphorylated AMPK enzyme; (iii) expression of mitochondrial uncoupling protein 3 (UCP3); (iv) free fatty acid level and/or fatty acid oxidation; (v) fat content; (vi) lean muscle mass, (vii) muscle thermogenesis; (viii) feeding behavior; (ix) the level of an insulin receptor (IR); (x) whole body energy expenditure; (xi) metabolic rate; and (xii) glucose tolerance (b) determining the said Cbl-associated phenotype in a non-human animal, isolated tissue or isolated cell having reduced expression of functional Cbl compared to an otherwise isogenic wild-type animal, tissue or cell; and (c) comparing the said Cbl-associated phenotype at (a) and (b) wherein a phenotype associated with Cbl-deficiency at (a) selected from the group consisting of (i) increased activity of acetyl CoA carboxylase (ACC) and/or amount of phosphorylated ACC enzyme; (ii) increased activity of AMP-dependent protein kinase (AMPK) and/or amount of phosphorylated AMPK enzyme; (iii) increased expression of UCP3; (iv) reduced free fatty acid level and/or increased fatty acid oxidation; (v) reduced fat content; (vi) increased lean muscle mass; (vii) increased muscle thermogenesis; (viii) increased feeding behavior; (ix) increased level of an insulin receptor (IR); (x) increased whole body energy expenditure: (xi) increased metabolic rate; and (xii) increased glucose tolerance indicates that the compound enhances whole body insulin sensitivity of an animal.

25. The method of claim 24 wherein ACC activity is determined by a process comprising by measuring the incorporation of labeled carbon into malonyl CoA in the presence and absence of the compound.

26. The method of claim 24 wherein phosphorylated ACC is determined by contacting cells or a cell extract with an antibody that binds to the phosphorylated form of the enzyme under conditions sufficient for an antigen-antibody complex to form and detecting the antibody bound.

27. The method of claim 26 further comprising separately contacting cells or cell extract with an antibody that binds to non-phosphorylated ACC under conditions sufficient for an antigen-antibody complex to form and detecting the antibody bound.

28. The method of claim 26 wherein detecting the antibody bound comprises contacting the antibody with a secondary antibody that is capable of producing a detectable signal.

29. The method of claim 24 further comprising determining the percentage of phosphorylated ACC relative to total ACC in the sample in the presence and absence of the compound being tested.

30. The method according to claim 24 wherein the non-human animal is a mammal.

31. The method according to claim 30 wherein the mammal is selected from the group consisting of rodent, dog, pig, bovine, sheep, horse and goat.

32. The method according to claim 31 wherein the rodent is selected from the group consisting of rabbit, rat, guinea pig and mouse.

33. The method according to claim 32 wherein the rodent is a mouse.

34. The method of claim 24 wherein the cells are skeletal muscle cells, cardiac myoblasts or adipocytes.

35. The method according to claim 24 wherein the animal, tissue or cell expresses an endogenous functional Cbl.

36. The method of claim 24 wherein the animal, tissue or cell expresses and introduced Cbl gene.

37. The method of claim 36 wherein the animal, tissue or cell is non-human and expresses an introduced human Cbl gene.

38. The method according to claim 24 wherein the compound being tested is a peptidyl inhibitor of Cbl.

39. The method of claim 38 wherein the inhibitor is a dominant negative mutant of Cbl.

40. (canceled)

41. The method according to claim 24 wherein the compound being tested comprises nucleic acid.

42-43. (canceled)

44. The method according to claim 24 wherein the compound is administered to muscle tissue of an animal subject.

45. The method of claim 24 further comprising:
(i) optionally, determining the structure of the compound; and
(ii) providing the compound or the name or structure of the compound.

46. The method of claim 45 comprising providing the compound or the name or structure of the compound with an indication as to its use.

47. The method of claim 45 further comprising producing or synthesizing the compound.

48-71. (canceled)

72. The method of claim 24 wherein AMPK activity is determined by a process comprising following the incorporation of labelled phosphate into a synthetic peptide SAMS in the presence and absence of the compound.

73. The method of claim 24 wherein phosphorylated AMPK is determined by contacting cells or a cell extract with an antibody that binds to the phosphorylated form of the enzyme under conditions sufficient for an antigen-antibody complex to form and detecting the antibody bound.

74. The method of claim 73 further comprising separately contacting cells or cell extract with an antibody that binds to non-phosphorylated AMPK under conditions sufficient for an antigen-antibody complex to form and detecting the antibody bound.

75. The method of claim 73 wherein detecting the antibody bound comprises contacting the antibody with a secondary antibody that is capable of producing a detectable signal.

76. The method of claim 24 further comprising determining the percentage of phosphorylated AMPK relative to total AMPK in the sample in the presence and absence of the compound being tested.

77-96. (canceled)

97. A method of identifying a therapeutic target for the treatment of aberrant insulin action or a condition associated therewith, said method comprising administering to an animal, tissue or cell a compound capable of reducing the expression and/or activity of a Cbl protein and determining the activity and/or expression of one or more genes and/or proteins in the animal, tissue or cell wherein modified expression and/or activity of a gene or protein indicates that the gene or protein is a therapeutic target for the treatment of aberrant insulin action or a condition associated therewith.

98-112. (canceled)

113. The method of claim 97 further comprising identifying a compound that modulates the expression or activity of the therapeutic target.

114. A method of identifying a compound that enhances free fatty acid synthesis or reduces a member selected from the group consisting of: (i) mitochondrial acetyl CoA carboxylase (ACC) enzyme activity; (ii) mitochondrial AMP kinase (AMPK) enzyme activity; (iii) expression of mitochondrial uncoupling protein 3 (UCP3); (iv) fatty acid oxidation; and (v) expression of an insulin receptor (IR), said method comprising determining the level of Cbl expression and/or activity in the presence and absence of the compound wherein enhanced expression and/or activity of Cbl in the presence of the compound indicates that the compound enhances free fatty acid synthesis or reduces a said member.

115. A method of identifying a compound that reduces free fatty acid synthesis or enhances a member selected from the group consisting of: (i) mitochondrial acetyl CoA carboxylase (ACC) enzyme activity; (ii) mitochondrial AMP kinase (AMPK) enzyme activity: (iii) expression of mitochondrial uncoupling protein 3 (UCP3); (iv) fatty acid oxidation: and (v) expression of an insulin receptor (IR) said method comprising determining the level of Cbl expression and/or activity in the presence and absence of the compound wherein reduced expression and/or activity of Cbl in the presence of the compound indicates that the compound reduces free fatty acid synthesis or enhances a said member.

116-125. (canceled)

126. The method according claim 114 wherein Cbl expression or activity is determined by a process comprising performing an immunoassay.

127. The method according to claim 114 comprising determining the amount of Cbl protein in the cell in the presence and absence of the compound.

128. The method of claim 114 comprising determining the level of c-Cbl-mediated ubiquitination of the insulin receptor in the presence and absence of the compound.

129. The method according to claim 114 wherein Cbl expression or activity is determined by a process comprising determining phosphorylation of a tyrosine residue on Cbl protein in the presence and absence of the compound.

130. The method according claim 115 wherein Cbl expression or activity is determined by a process comprising performing an immunoassay.

131. The method according to claim 115 comprising determining the amount of Cbl protein in the cell in the presence and absence of the compound.

132. The method of claim 115 comprising determining the level of c-Cbl-mediated ubiquitination of the insulin receptor in the presence and absence of the compound.

133. The method according to claim 115 wherein Cbl expression or activity is determined by a process comprising determining phosphorylation of a tyrosine residue on Cbl protein in the presence and absence of the compound.

134. A method for identifying a compound that enhances insulin sensitivity and/or energy expenditure of an animal comprising determining the level of Cbl expression and/or activity in the presence and absence of the compound wherein reduced expression and/or activity of Cbl in the presence of the compound indicates that the compound enhances insulin sensitivity and/or energy expenditure.

135. The method according claim 134 wherein Cbl expression or activity is determined by a process comprising performing an immunoassay.

136. The method according to claim 134 comprising determining the amount of Cbl protein in the cell in the presence and absence of the compound.

137. The method of claim 134 comprising determining the level of c-Cbl-mediated ubiquitination of the insulin receptor in the presence and absence of the compound.

138. The method according to claim 134 wherein Cbl expression or activity is determined by a process comprising determining phosphorylation of a tyrosine residue on Cbl protein in the presence and absence of the compound.

139. A method for identifying a compound that reduces insulin sensitivity and/or energy expenditure of an animal comprising determining the level of Cbl expression and/or activity in the presence and absence of the compound wherein reduced expression and/or activity of Cbl in the presence of the compound indicates that the compound reduces insulin sensitivity and/or energy expenditure.

140. The method according claim 139 wherein Cbl expression or activity is determined by a process comprising performing an immunoassay.

141. The method according to claim 139 comprising determining the amount of Cbl protein in the cell in the presence and absence of the compound.

142. The method of claim 139 comprising determining the level of c-Cbl-mediated ubiquitination of the insulin receptor in the presence and absence of the compound.

143. The method according to claim 139 wherein Cbl expression or activity is determined by a process comprising determining phosphorylation of a tyrosine residue on Cbl protein in the presence and absence of the compound.

144. A method of treatment of an animal or human subject for a condition associated with aberrant insulin action said method comprising administering to the subject an effective amount of a compound that modulates ACC activity and/or AMPK activity and/or FFA level and/or fatty acid oxidation and/or Cbl expression or activity thereby treating a condition associated with aberrant insulin action.

145. The method of claim 144 wherein the condition is selected form the group consisting of hyperglycemia, hyperinsulinemia, obesity, adult-onset obesity, non-insulin-dependent diabetes mellitus, type II diabetes, glucose intolerance, hypertrophy or hyperplasia of the islets of Langerhans, atherosclerosis and heart disease.


SEQUENCE LISTING (268 sequences)

SEQ ID NO: 1 - protein, 153 aa, Mus musculus
MAGNVKKSSGAGGGGSGGSGAGGLIGLMKDAFQPHHHHHHLSPHPPCTVDKKMVEKCWKLMDKVVRLCQNPNVALKNSPPYILDLLPDTYQHLRTVLSRYEGKMETLGENEYFRVFMENLMKKTKQTISLFKEGKERMYEENSQPRRNLTKLS

SEQ ID NO: 2 - protein, 896 aa, Mus musculus
MAGNVKKSSGAGGGGSGGSGAGGLIGLMKDAFQPHHHHHHLSPHPPCTVDKKMVEKCWKLMDKVVRLCQNPNVALKNSPPYILDLLPDTYQHLRTVLSRYEGKMETLGENEYFRVFMENLMKKTKQTISLFKEGKERMYEENSQPRRNLTKLSLIFSHMLAELKGIFPSGLFQGDTFRITKADAAEFWRKAFGEKTIVPWKSFRQALHEVHPISSGLEAMALKSTIDLTCNDYISVFEFDIFTRLFQPWSSLLRNWNSLAVTHPGYMAFLTYDEVKARLQKFIHKPGSYIFRLSCTRLGQWAIGYVTADGNILQTIPHNKPLFQALIDGFREGFYLFPDGRNQNPDLTGLCEPTPQDHIKVTQICAENDKDVKIEPCGHLMCTSCLTSWQESEGQGCPFCRCEIKGTEPIVVDPFDPRGSGSLLRQGAEGAPSPNYDDDDDERADDSLFMMKELAGAKVERPSSPFSMAPQASLPPVPPRLDLLQQRAPVPASTSVLGTASKAASGSLHKDKPLPIPPTLRDLPPPPPPDRPYSVGAETRPQRRPLPCTPGDCPSRDKLPPVPSSRPGDSWLSRTIPKVPVATPNPGDPWNGRELTNRHSLPFSLPSQMEPRADVPRLGSTFSLDTSMTMNSSPVAGPESEHPKIKPSSSANAIYSLAARPLPMPKLPPGEQGESEEDTEYMTPTSRPVGVQKPEPKRPLEATQSSRACDCDQQIDSCTYEAMYTIQSQALSVAENSASGEGNLATAHTSTGPEESENEDDGYDVPKPPVPAVLARRTLSDISNASSSFGWLSLDGDPTNFNEGSQVPERPPKPFPRRINSERKASSYQQGGGATANPVATAPSPQLSSEIERLMSQGYSYQDIQKALVIAHNNIEMAKNILREFVSISSPAHVAT

SEQ ID NO: 3 - protein, 906 aa, Homo sapiens
MAGNVKKSSGAGGGTGSGGSGSGGLIGLMKDAFQPHHHHHHHLSPHPPGTVDKKMVEKCWKLMDKVVRLCQNPKLALKNSPPYILDLLPDTYQHLRTILSRYEGKMETLGENEYFRVFMENLMKKTKQTISLFKEGKERMYEENSQPRRNLTKLSLIFSHMLAELKGIFPSGLFQGDTFRITKADAAEFWRKAFGEKTIVPWKSFRQALHEVHPISSGLEAMALKSTIDLTCNDYISVFEFDIFTRLFQPWSSLLRNWNSLAVTHPGYMAFLTYDEVKARLQKFIHKPGSYIFRLSCTRLGQWAIGYVTADGNILQTIPHNKPLFQALIDGFREGFYLFPDGRNQNPDLTGLCEPTPQDHIKVTQEQYELYCEMGSTFQLCKICAENDKDVKIEPCGHLMCTSCLTSWQESEGQGCPFCRCEIKGTEPIVVDPFDPRGSGSLLRQGAEGAPSPNYDDDDDERADDTLFMMKELAGAKVERPPSPFSMAPQASLPPVPPRLDLLPQRVCVPSSASALGTASKAASGSLHKDKPLPVPPTLRDLPPPPPPDRPYSVGAESRPQRRPLPCTPGDCPSRDKLPPVPSSRLGDSWLPRPIPKVPVSAPSSSDPWTGRELTNRHSLPFSLPSQMEPRPDVPRLGSTFSLDTSMSMNSSPLVGPECDHPKIKPSSSANAIYSLAARPLPVPKLPPGEQCEGEEDTEYMTPSSRPLRPLDTSQSSRACDCDQQIDSCTYEAMYNIQSQAPSITESSTFGEGNLAAAHANTGPEESENEDDGYDVPKPPVPAVLARRTLSDISNASSSFGWLSLDGDPTTNVTEGSQVPERPPKPFPRRINSERKAGSCQQGSGPAASAATASPQLSSEIENLMSQGYSYQDIQKALVIAQNNIEMAKNILREFVSISSPAHVAT

SEQ ID NO: 4 - protein, 2483 aa, homo sapiens
MVLLLCLSCLIFSCLTFSWLKIWEKMTDSKPITKSKSEANLIPSQEPFPASDNSGETPQRNGEGHTLHKDTQPGRAQPPTKAQRSGRRRNSLPPSRQKPPRNPLSSSDAAPSPELQANGTGTQGLEATDTNGLSSSARPQGSKLVPSKEDKKQANIKRQLMTNFILGSFDDYSSDEDSVAGSSRESTRKGSRASLGALSLEAYLTTGEAETRVPTMRPSMSGLHLVKRGREHKKLDLHRDFTVASPAEFVTRFGGDRVIEKVLIANNGIAAVKCMRSIRRWAYEMFRNERAIRFVRMVTPEDLKANAEYIKMADHYGPAPGGPNNNNYANVELIVDIAKRIPLQAVWAGWGHALENPKLPELLCKNGVAFLGPPRLRPMVGLGDKIASTVVAQTLQVPTLPRSGSALTVEWTEDDLQQGKRISVPEDVYDKGCVKDVDEGLEAAERIGFPLMIKASEGGGGKGIRETESAEDFPILFRQVQSEIPGSPIFLMKLAQHARHLEVQILADQYGNAVSLFGRDCSIQRRHQKIVEEAPATIAPLAIFEFMEQCAIRLAKTVGYVSAGTVEYLYSQDGSFHFLELNPRLQVEHPCTEMIADVNLPAAQLQIAMGAPLHRLKDIRLLYGESPWGDSPISFENSAHLPCPRGHVIATRITSENPDEGFKPSSGTVQELNFRSSKNVWGYFTVAATGGLHEFAISQFGHCFSWGENRKEAISNMVVALKELSLRGDFRTTVEYLINLLETESFQNNYIDTGWLDYLIAEKVQKKPNIMLGVVCGALERGDAMFRTCMTDFLHSLERGQVLPADSLLNLVDVELIYEGVKYILKVTRQSLTMFVLIMNGCHIEIDAHRLNDGGLLLSYNGNSYTTYMKEEVDSYRTIGNKTCVFEKENDPTVLRSPSAGKLTQITVEDGGHVEAGRRYAEMEVMKMIMTLNVQERGRVKYIKRPGAVLEAGCVVARLELDDPSKVHPAEPFTGELPAQQNTADLGKKLHRVFHSVLGSLTNVMSGFCLPEPFFSIKLKEWVQKLMMTLRHPSLLLDVQEIMTSRAGRIPPPVEKSVRKVMAQYASNITSVLCQFPSQQIATILDCHAATLQRKADREVFFINTQSMVQLVQRYRSGIRGHMKTVVIDLLRRYLRVETIFGKARDADANSSGMVGGVRSLSFTSVWVVLSPPAHYDKCVINLREQFKPDMSQVLDCIFSHAQVTKKNQLVIMLIDELCGPDPSLSDELISILNELTQLSKSEHCKVALRARQILIASPSYELRHNQVESIFLSAIDMYGHQFCPENLQKLILSETTIFDVLNTFFYHANKVVCMASLEVYVGGAYIAYVLNSLQHRQLPDGTCVVEFQFMLPSSHPNRMTVPISITNPDLLRHTTELFMDSGFSPLCQRMGAMVAFRRFEDFTRNFDEVISCFANVPKDPPLFSEARTSLYSEDDCKSLREEPIHILNVSIQCADHLEDEALVPILRTFVQSKKNILVDYGLRRIPFLIAQEKEFPKFFTFRARDEFAEDRIYRHLEPALAFQLELNRMRNFDLTAVPCANHKMHLYLGAAKVEGRYEVTDHRFFIRAIIRHSDLITKEASFEYLQNEGERLLLEAMDELEVAFNNTNVRTDCNHIFLNFVPTVIMDPNKIEESVRYMVMRYGSRLWKLRVLQAEVKINIRQTTTGSAVPIRLFITNESGYYLDISLYKEVTDSRSGNIMFHSFGNKQGPQHGMLINTPYVTKDLLQAKRFQAQTLGTTYIYDFPEMFRQALFKLWGSPDKYPKDILTYTELVLDSQGQLVEMNRLPGGNEVGMVAFKMRFKTQEYPEGRDVIVIGNDITFRIGSFGPGEDLLYLRASEMARAEAIPKIYVAANSGARIGMAEEIKHMFHVAWVDPEDPHKGFKYLYLTPQDYTRISSLNSVHCKHIEEGGESRYMITDIIGKDDGLGVENLRGSGMIAGESSLAYEEIVTISLVTCRAIGIGAYLVRLGQRVIQVENSHIILTGASALNKVLGREVYTSNNQLGGVQIMHYNGVSHITVPDDFEGVYTILEWLSYMPKDNHSPVPIITPTDPIDREIEFLPSRAPYDPRWMLAGRPHPTLKGTWQSGFFDHGSFKEIMAPWAQTVVTGRARLGGIPVGVIAVETRTVEVAVPADPANLDSEAKIIQQAGQVWFPDSAYKTAQAIKDFNREKLPLMIFANWRGFSGGMKDMYDQVLKFGAYIVDGLRQYKQPILIYIRPMRELRGGSWVVIDATINPLCIEMYADKESRGGVLEPEGTVEIKFRKEDLIKSMRRIDPAYKKLMEQLGEPDLSDKDRKDLEGRLKAREDLLLPIYHQVAVQFADFHDTPGRMLEKGVISDILEWKTARTFLYWRLRRLLLEDQVKQEILQASGELSHVHIQSMLRRWFVETEGAVKAYLWDNNQVVVQWLEQHWQAGDGPRSTIRENITYLKHDSVLKTIRGLVEENPEVAVDCVIYLSQHISPAERAQVVHLLSTMDSPAST

SEQ ID NO: 5 - protein, 552 aa, homo sapiens
MAEKQKHDGRVKIGHYVLGDTLGVGTFGKVKIGEHQLTGHKVAVKILNRQKIRSLDVVGKIKREIQNLKLFRHPHIIKLYQVISTPTDFFMVMEYVSGGELFDYICKHGRVEEMEARRLFQQILSAVDYCHRHMVVHRDLKPENVLLDAHMNAKIADFGLSNMMSDGEFLRTSCGSPNYAAPEVISGRLYAGPEVDIWSCGVILYALLCGTLPFDDEHVPTLFKKIRGGVFYIPEYLNRSVATLLMHMLQVDPLKRATIKDIREHEWFKQDLPSYLFPEDPSYDANVIDDEAVKEVCEKFECTESEVMNSLYSGDPQDQLAVAYHLIIDNRRIMNQASEFYLASSPPSGSFMDDSAMHIPPGLKPHPERMPPLIADSPKARCPLDALNTTKPKSLAVKKAKWHLGIRSQSKPYDIMAEVYRAMKQLDFEWKVVNAYHLRVRRKNPVTGNYVKMSLQLYLVDNRSYLLDFKSIDDEVVEQRSGSSTPQRSCSAAGLHRPRSSFDSTTAESHSLSGSLTGSLTGSTLSSVSPRLGSHTMDFFEMCASLITTLAR

SEQ ID NO: 6 - DNA, 21 bp, Artificial
  siRNA sense strand oligonucleotide
cgtgaagaagagctctgggtt

SEQ ID NO: 7 - DNA, 21 bp, Artificial
  siRNA sense strand oligonucleotide
gaagatggtggagaagtgctt

SEQ ID NO: 8 - DNA, 21 bp, Artificial
  siRNA sense strand oligonucleotide
gatggtggagaagtgctggtt

SEQ ID NO: 9 - DNA, 21 bp, Artificial
  siRNA sense strand oligonucleotide
gtgctggaagctcatggactt

SEQ ID NO: 10 - DNA, 21 bp, Artificial
  siRNA sense strand oligonucleotide
gctcatggacaaggtggtgtt

SEQ ID NO: 11 - DNA, 21 bp, Artificial
  siRNA sense strand oligonucleotide
ggtggtgcggttgtgtcagtt

SEQ ID NO: 12 - DNA, 21 bp, Artificial
  siRNA sense strand oligonucleotide
cccaaagctggcgctaaagtt

SEQ ID NO: 13 - DNA, 21 bp, Artificial
  siRNA sense strand oligonucleotide
cccaaagctggcgctaaagtt

SEQ ID NO: 14 - DNA, 21 bp, Artificial
  siRNA sense strand oligonucleotide
agctggcgctaaagaatagtt

SEQ ID NO: 15 - DNA, 21 bp, Artificial
  siRNA sense strand oligonucleotide
agaatagcccaccttatattt

SEQ ID NO: 16 - DNA, 21 bp, Artificial
  siRNA sense strand oligonucleotide
tagcccaccttatatcttatt

SEQ ID NO: 17 - DNA, 21 bp, Artificial
  siRNA sense strand oligonucleotide
gatatgaggggaagatggatt

SEQ ID NO: 18 - DNA, 21 bp, Artificial
  siRNA sense strand oligonucleotide
gatggagacacttggagaatt

SEQ ID NO: 19 - DNA, 21 bp, Artificial
  siRNA sense strand oligonucleotide
ctaagcaaaccataagccttt

SEQ ID NO: 20 - DNA, 21 bp, Artificial
  siRNA sense strand oligonucleotide
gcaaaccataagcctcttctt

SEQ ID NO: 21 - DNA, 21 bp, Artificial
  siRNA sense strand oligonucleotide
accataagcctcttcaaggtt

SEQ ID NO: 22 - DNA, 21 bp, Artificial
  siRNA sense strand oligonucleotide
gcctcttcaaggagggaaatt

SEQ ID NO: 23 - DNA, 21 bp, Artificial
  siRNA sense strand oligonucleotide
gaaagaatgtatgaggagatt

SEQ ID NO: 24 - DNA, 21 bp, Artificial
  siRNA sense strand oligonucleotide
agaatgtatgaggagaatttt

SEQ ID NO: 25 - DNA, 21 bp, Artificial
  siRNA sense strand oligonucleotide
tgtatgaggagaattctcatt

SEQ ID NO: 26 - DNA, 21 bp, Artificial
  siRNA sense strand oligonucleotide
ttctcagcctaggcgaaactt

SEQ ID NO: 27 - DNA, 21 bp, Artificial
  siRNA sense strand oligonucleotide
acctaaccaaactgtcccttt

SEQ ID NO: 28 - DNA, 21 bp, Artificial
  siRNA sense strand oligonucleotide
ccaaactgtccctcatctttt

SEQ ID NO: 29 - DNA, 21 bp, Artificial
  siRNA sense strand oligonucleotide
actgtccctcatcttcagctt

SEQ ID NO: 30 - DNA, 21 bp, Artificial
  siRNA sense strand oligonucleotide
ggaatctttccaagtggactt

SEQ ID NO: 31 - DNA, 21 bp, Artificial
  siRNA sense strand oligonucleotide
tctttccaagtggactctttt

SEQ ID NO: 32 - DNA, 21 bp, Artificial
  siRNA sense strand oligonucleotide
gtggactctttcagggagatt

SEQ ID NO: 33 - DNA, 21 bp, Artificial
  siRNA sense strand oligonucleotide
agcagatgctgcggaattttt

SEQ ID NO: 34 - DNA, 21 bp, Artificial
  siRNA sense strand oligonucleotide
gacaatagtcccttggaagtt

SEQ ID NO: 35 - DNA, 21 bp, Artificial
  siRNA sense strand oligonucleotide
tagtcccttggaagagctttt

SEQ ID NO: 36 - DNA, 21 bp, Artificial
  siRNA sense strand oligonucleotide
gagctttcgacaggctctatt

SEQ ID NO: 37 - DNA, 21 bp, Artificial
  siRNA sense strand oligonucleotide
gtgcatcccatcagttctgtt

SEQ ID NO: 38 - DNA, 21 bp, Artificial
  siRNA sense strand oligonucleotide
atccactattgatctgacctt

SEQ ID NO: 39 - DNA, 21 bp, Artificial
  siRNA sense strand oligonucleotide
tttgacatctttacccgactt

SEQ ID NO: 40 - DNA, 21 bp, Artificial
  siRNA sense strand oligonucleotide
ttggaacagccttgctgtatt

SEQ ID NO: 41 - DNA, 21 bp, Artificial
  siRNA sense strand oligonucleotide
cagccttgctgtaactcattt

SEQ ID NO: 42 - DNA, 21 bp, Artificial
  siRNA sense strand oligonucleotide
ctcatcctggctacatggctt

SEQ ID NO: 43 - DNA, 21 bp, Artificial
  siRNA sense strand oligonucleotide
gtgaaagctcggctccagatt

SEQ ID NO: 44 - DNA, 21 bp, Artificial
  siRNA sense strand oligonucleotide
agctcggctccagaaattctt

SEQ ID NO: 45 - DNA, 21 bp, Artificial
  siRNA sense strand oligonucleotide
attcattcacaaacctggctt

SEQ ID NO: 46 - DNA, 21 bp, Artificial
  siRNA sense strand oligonucleotide
acctggcagttatatcttctt

SEQ ID NO: 47 - DNA, 21 bp, Artificial
  siRNA sense strand oligonucleotide
cattctccagacaatcccttt

SEQ ID NO: 48 - DNA, 21 bp, Artificial
  siRNA sense strand oligonucleotide
tccctcacaataaacctcttt

SEQ ID NO: 49 - DNA, 21 bp, Artificial
  siRNA sense strand oligonucleotide
taaacctctcttccaagcatt

SEQ ID NO: 50 - DNA, 21 bp, Artificial
  siRNA sense strand oligonucleotide
acctctcttccaagcactgtt

SEQ ID NO: 51 - DNA, 21 bp, Artificial
  siRNA sense strand oligonucleotide
gcactgattgatggcttcatt

SEQ ID NO: 52 - DNA, 21 bp, Artificial
  siRNA sense strand oligonucleotide
ggcttctatttgtttcctgtt

SEQ ID NO: 53 - DNA, 21 bp, Artificial
  siRNA sense strand oligonucleotide
atcagaatcctgatctgactt

SEQ ID NO: 54 - DNA, 21 bp, Artificial
  siRNA sense strand oligonucleotide
tcctgatctgactggcttatt

SEQ ID NO: 55 - DNA, 21 bp, Artificial
  siRNA sense strand oligonucleotide
ccaactccccaagaccatatt

SEQ ID NO: 56 - DNA, 21 bp, Artificial
  siRNA sense strand oligonucleotide
ctccccaagaccatatcaatt

SEQ ID NO: 57 - DNA, 21 bp, Artificial
  siRNA sense strand oligonucleotide
gaccatatcaaagtgaccctt

SEQ ID NO: 58 - DNA, 21 bp, Artificial
  siRNA sense strand oligonucleotide
agtgacccaggaacaatattt

SEQ ID NO: 59 - DNA, 21 bp, Artificial
  siRNA sense strand oligonucleotide
caatatgaattatactgtgtt

SEQ ID NO: 60 - DNA, 21 bp, Artificial
  siRNA sense strand oligonucleotide
tatgaattatactgtgagatt

SEQ ID NO: 61 - DNA, 21 bp, Artificial
  siRNA sense strand oligonucleotide
ttatactgtgagatgggcttt

SEQ ID NO: 62 - DNA, 21 bp, Artificial
  siRNA sense strand oligonucleotide
tgataaggatgtaaagatttt

SEQ ID NO: 63 - DNA, 21 bp, Artificial
  siRNA sense strand oligonucleotide
ggatgtaaagattgagccctt

SEQ ID NO: 64 - DNA, 21 bp, Artificial
  siRNA sense strand oligonucleotide
agattgagccctgtggacatt

SEQ ID NO: 65 - DNA, 21 bp, Artificial
  siRNA sense strand oligonucleotide
tcagaaggtcagggctgtctt

SEQ ID NO: 66 - DNA, 21 bp, Artificial
  siRNA sense strand oligonucleotide
ggtcagggctgtcctttcttt

SEQ ID NO: 67 - DNA, 21 bp, Artificial
  siRNA sense strand oligonucleotide
attaaaggtactgaacccatt

SEQ ID NO: 68 - DNA, 21 bp, Artificial
  siRNA sense strand oligonucleotide
aggtactgaacccatcgtgtt

SEQ ID NO: 69 - DNA, 21 bp, Artificial
  siRNA sense strand oligonucleotide
cccatcgtggtagatccgttt

SEQ ID NO: 70 - DNA, 21 bp, Artificial
  siRNA sense strand oligonucleotide
attatgatgatgatgatgatt

SEQ ID NO: 71 - DNA, 21 bp, Artificial
  siRNA sense strand oligonucleotide
cgagctgatgatactctcttt

SEQ ID NO: 72 - DNA, 21 bp, Artificial
  siRNA sense strand oligonucleotide
ggaattggctggtgccaagtt

SEQ ID NO: 73 - DNA, 21 bp, Artificial
  siRNA sense strand oligonucleotide
ttggctggtgccaaggtggtt

SEQ ID NO: 74 - DNA, 21 bp, Artificial
  siRNA sense strand oligonucleotide
cggccgccttctccattcttt

SEQ ID NO: 75 - DNA, 21 bp, Artificial
  siRNA sense strand oligonucleotide
gtgcttctgctcttggaactt

SEQ ID NO: 76 - DNA, 21 bp, Artificial
  siRNA sense strand oligonucleotide
ctgcttctaaggctgcttctt

SEQ ID NO: 77 - DNA, 21 bp, Artificial
  siRNA sense strand oligonucleotide
ggctgcttctggctccctttt

SEQ ID NO: 78 - DNA, 21 bp, Artificial
  siRNA sense strand oligonucleotide
agacaaaccattgccagtatt

SEQ ID NO: 79 - DNA, 21 bp, Artificial
  siRNA sense strand oligonucleotide
accattgccagtacctccctt

SEQ ID NO: 80 - DNA, 21 bp, Artificial
  siRNA sense strand oligonucleotide
tcccgacctcaaagacgcctt

SEQ ID NO: 81 - DNA, 21 bp, Artificial
  siRNA sense strand oligonucleotide
agacgccccttgccttgtatt

SEQ ID NO: 82 - DNA, 21 bp, Artificial
  siRNA sense strand oligonucleotide
tccccaaagtaccagtatctt

SEQ ID NO: 83 - DNA, 21 bp, Artificial
  siRNA sense strand oligonucleotide
agtaccagtatctgccccatt

SEQ ID NO: 84 - DNA, 21 bp, Artificial
  siRNA sense strand oligonucleotide
gttccagtgatccctggactt

SEQ ID NO: 85 - DNA, 21 bp, Artificial
  siRNA sense strand oligonucleotide
gagaattaaccaaccggcatt

SEQ ID NO: 86 - DNA, 21 bp, Artificial
  siRNA sense strand oligonucleotide
ttaaccaaccggcactcactt

SEQ ID NO: 87 - DNA, 21 bp, Artificial
  siRNA sense strand oligonucleotide
ccaaccggcactcacttcctt

SEQ ID NO: 88 - DNA, 21 bp, Artificial
  siRNA sense strand oligonucleotide
ccggcactcacttccattttt

SEQ ID NO: 89 - DNA, 21 bp, Artificial
  siRNA sense strand oligonucleotide
atggagcccagaccagatgtt

SEQ ID NO: 90 - DNA, 21 bp, Artificial
  siRNA sense strand oligonucleotide
gcacgttcagtctggatactt

SEQ ID NO: 91 - DNA, 21 bp, Artificial
  siRNA sense strand oligonucleotide
tagcagcccattagtaggttt

SEQ ID NO: 92 - DNA, 21 bp, Artificial
  siRNA sense strand oligonucleotide
tcaaaccttcctcatctgctt

SEQ ID NO: 93 - DNA, 21 bp, Artificial
  siRNA sense strand oligonucleotide
accttcctcatctgccaattt

SEQ ID NO: 94 - DNA, 21 bp, Artificial
  siRNA sense strand oligonucleotide
tgccatttattctctggcttt

SEQ ID NO: 95 - DNA, 21 bp, Artificial
  siRNA sense strand oligonucleotide
ctgccacctggggagcaattt

SEQ ID NO: 96 - DNA, 21 bp, Artificial
  siRNA sense strand oligonucleotide
tgtgagggtgaagaggacatt

SEQ ID NO: 97 - DNA, 21 bp, Artificial
  siRNA sense strand oligonucleotide
gaggacacagagtacatgatt

SEQ ID NO: 98 - DNA, 21 bp, Artificial
  siRNA sense strand oligonucleotide
gcaatgtataatattcagttt

SEQ ID NO: 99 - DNA, 21 bp, Artificial
  siRNA sense strand oligonucleotide
tgtataatattcagtcccatt

SEQ ID NO: 100 - DNA, 21 bp, Artificial
  siRNA sense strand oligonucleotide
tattcagtcccaggcgccatt

SEQ ID NO: 101 - DNA, 21 bp, Artificial
  siRNA sense strand oligonucleotide
cactggtcccgaggagtcatt

SEQ ID NO: 102 - DNA, 21 bp, Artificial
  siRNA sense strand oligonucleotide
tgaggatgatgggtatgattt

SEQ ID NO: 103 - DNA, 21 bp, Artificial
  siRNA sense strand oligonucleotide
ctctctcagatatctctaatt

SEQ ID NO: 104 - DNA, 21 bp, Artificial
  siRNA sense strand oligonucleotide
tgccagctcctcctttggctt

SEQ ID NO: 105 - DNA, 21 bp, Artificial
  siRNA sense strand oligonucleotide
caaatgtcactgaaggttctt

SEQ ID NO: 106 - DNA, 21 bp, Artificial
  siRNA sense strand oligonucleotide
atgtcactgaaggttcccatt

SEQ ID NO: 107 - DNA, 21 bp, Artificial
  siRNA sense strand oligonucleotide
ggttcccaagttcccgagatt

SEQ ID NO: 108 - DNA, 21 bp, Artificial
  siRNA sense strand oligonucleotide
gttcccgagaggcctccaatt

SEQ ID NO: 109 - DNA, 21 bp, Artificial
  siRNA sense strand oligonucleotide
ccattcccgcggagaatcatt

SEQ ID NO: 110 - DNA, 21 bp, Artificial
  siRNA sense strand oligonucleotide
tcaactctgaacggaaagctt

SEQ ID NO: 111 - DNA, 21 bp, Artificial
  siRNA sense strand oligonucleotide
ctctgaacggaaagctggctt

SEQ ID NO: 112 - DNA, 21 bp, Artificial
  siRNA sense strand oligonucleotide
cggaaagctggcagctgtctt

SEQ ID NO: 113 - DNA, 21 bp, Artificial
  siRNA sense strand oligonucleotide
agctggcagctgtcagcaatt

SEQ ID NO: 114 - DNA, 21 bp, Artificial
  siRNA sense strand oligonucleotide
cctcatgagtcaggggtactt

SEQ ID NO: 115 - DNA, 21 bp, Artificial
  siRNA sense strand oligonucleotide
agctttggtcattgcccagtt

SEQ ID NO: 116 - DNA, 21 bp, Artificial
  siRNA sense strand oligonucleotide
caacatcgagatggccaaatt

SEQ ID NO: 117 - DNA, 21 bp, Artificial
  siRNA sense strand oligonucleotide
acatcctccgggaatttgttt

SEQ ID NO: 118 - DNA, 21 bp, Artificial
  siRNA sense strand oligonucleotide
tttgtttccatttcttctctt

SEQ ID NO: 119 - DNA, 21 bp, Artificial
  siRNA sense strand oligonucleotide
gtggcacctagaagggcagtt

SEQ ID NO: 120 - DNA, 21 bp, Artificial
  siRNA sense strand oligonucleotide
gggcaggagttcctttggttt

SEQ ID NO: 121 - DNA, 21 bp, Artificial
  siRNA sense strand oligonucleotide
gtcttgccctctctgtgggtt

SEQ ID NO: 122 - DNA, 21 bp, Artificial
  siRNA sense strand oligonucleotide
gatttcaaagtggtgaaattt

SEQ ID NO: 123 - DNA, 21 bp, Artificial
  siRNA sense strand oligonucleotide
tggagcagctagtatgttttt

SEQ ID NO: 124 - DNA, 21 bp, Artificial
  siRNA antisense strand oligonucleotide
cccagagctcttcttcacgtt

SEQ ID NO: 125 - DNA, 21 bp, Artificial
  siRNA antisense strand oligonucleotide
gcacttctccaccatcttctt

SEQ ID NO: 126 - DNA, 21 bp, Artificial
  siRNA antisense strand oligonucleotide
ccagcacttctccaccatctt

SEQ ID NO: 127 - DNA, 21 bp, Artificial
  siRNA antisense strand oligonucleotide
gtccatgagcttccagcactt

SEQ ID NO: 128 - DNA, 21 bp, Artificial
  siRNA antisense strand oligonucleotide
caccaccttgtccatgagctt

SEQ ID NO: 129 - DNA, 21 bp, Artificial
  siRNA antisense strand oligonucleotide
ctgacacaaccgcaccacctt

SEQ ID NO: 130 - DNA, 21 bp, Artificial
  siRNA antisense strand oligonucleotide
ctttagcgccagctttgggtt

SEQ ID NO: 131 - DNA, 21 bp, Artificial
  siRNA antisense strand oligonucleotide
ctttagcgccagctttgggtt

SEQ ID NO: 132 - DNA, 21 bp, Artificial
  siRNA antisense strand oligonucleotide
ctattctttagcgccagcttt

SEQ ID NO: 133 - DNA, 21 bp, Artificial
  siRNA antisense strand oligonucleotide
atataaggtgggctattcttt

SEQ ID NO: 134 - DNA, 21 bp, Artificial
  siRNA antisense strand oligonucleotide
taagatataaggtgggctatt

SEQ ID NO: 135 - DNA, 21 bp, Artificial
  siRNA antisense strand oligonucleotide
tccatcttcccctcatatctt

SEQ ID NO: 136 - DNA, 21 bp, Artificial
  siRNA antisense strand oligonucleotide
ttctccaagtgtctccatctt

SEQ ID NO: 137 - DNA, 21 bp, Artificial
  siRNA antisense strand oligonucleotide
aggcttatggtttgcttagtt

SEQ ID NO: 138 - DNA, 21 bp, Artificial
  siRNA antisense strand oligonucleotide
gaagaggcttatggtttgctt

SEQ ID NO: 139 - DNA, 21 bp, Artificial
  siRNA antisense strand oligonucleotide
ccttgaagaggcttatggttt

SEQ ID NO: 140 - DNA, 21 bp, Artificial
  siRNA antisense strand oligonucleotide
tttccctccttgaagaggctt

SEQ ID NO: 141 - DNA, 21 bp, Artificial
  siRNA antisense strand oligonucleotide
tctcctcatacattctttctt

SEQ ID NO: 142 - DNA, 21 bp, Artificial
  siRNA antisense strand oligonucleotide
aattctcctcatacattcttt

SEQ ID NO: 143 - DNA, 21 bp, Artificial
  siRNA antisense strand oligonucleotide
tgagaattctcctcatacatt

SEQ ID NO: 144 - DNA, 21 bp, Artificial
  siRNA antisense strand oligonucleotide
gtttcgcctaggctgagaatt

SEQ ID NO: 145 - DNA, 21 bp, Artificial
  siRNA antisense strand oligonucleotide
agggacagtttggttaggttt

SEQ ID NO: 146 - DNA, 21 bp, Artificial
  siRNA antisense strand oligonucleotide
aagatgagggacagtttggtt

SEQ ID NO: 147 - DNA, 21 bp, Artificial
  siRNA antisense strand oligonucleotide
gctgaagatgagggacagttt

SEQ ID NO: 148 - DNA, 21 bp, Artificial
  siRNA antisense strand oligonucleotide
gtccacttggaaagattcctt

SEQ ID NO: 149 - DNA, 21 bp, Artificial
  siRNA antisense strand oligonucleotide
aagagtccacttggaaagatt

SEQ ID NO: 150 - DNA, 21 bp, Artificial
  siRNA antisense strand oligonucleotide
tctccctgaaagagtccactt

SEQ ID NO: 151 - DNA, 21 bp, Artificial
  siRNA antisense strand oligonucleotide
aaattccgcagcatctgcttt

SEQ ID NO: 152 - DNA, 21 bp, Artificial
  siRNA antisense strand oligonucleotide
cttccaagggactattgtctt

SEQ ID NO: 153 - DNA, 21 bp, Artificial
  siRNA antisense strand oligonucleotide
aagctcttccaagggactatt

SEQ ID NO: 154 - DNA, 21 bp, Artificial
  siRNA antisense strand oligonucleotide
tagagcctgtcgaaagctctt

SEQ ID NO: 155 - DNA, 21 bp, Artificial
  siRNA antisense strand oligonucleotide
cagaactgatgggatgcactt

SEQ ID NO: 156 - DNA, 21 bp, Artificial
  siRNA antisense strand oligonucleotide
ggtcagatcaatagtggattt

SEQ ID NO: 157 - DNA, 21 bp, Artificial
  siRNA antisense strand oligonucleotide
gtcgggtaaagatgtcaaatt

SEQ ID NO: 158 - DNA, 21 bp, Artificial
  siRNA antisense strand oligonucleotide
tacagcaaggctgttccaatt

SEQ ID NO: 159 - DNA, 21 bp, Artificial
  siRNA antisense strand oligonucleotide
atgagttacagcaaggctgtt

SEQ ID NO: 160 - DNA, 21 bp, Artificial
  siRNA antisense strand oligonucleotide
gccatgtagccaggatgagtt

SEQ ID NO: 161 - DNA, 21 bp, Artificial
  siRNA antisense strand oligonucleotide
tctggagccgagctttcactt

SEQ ID NO: 162 - DNA, 21 bp, Artificial
  siRNA antisense strand oligonucleotide
gaatttctggagccgagcttt

SEQ ID NO: 163 - DNA, 21 bp, Artificial
  siRNA antisense strand oligonucleotide
gccaggtttgtgaatgaattt

SEQ ID NO: 164 - DNA, 21 bp, Artificial
  siRNA antisense strand oligonucleotide
gaagatataactgccaggttt

SEQ ID NO: 165 - DNA, 21 bp, Artificial
  siRNA antisense strand oligonucleotide
agggattgtctggagaatgtt

SEQ ID NO: 166 - DNA, 21 bp, Artificial
  siRNA antisense strand oligonucleotide
agaggtttattgtgagggatt

SEQ ID NO: 167 - DNA, 21 bp, Artificial
  siRNA antisense strand oligonucleotide
tgcttggaagagaggtttatt

SEQ ID NO: 168 - DNA, 21 bp, Artificial
  siRNA antisense strand oligonucleotide
cagtgcttggaagagaggttt

SEQ ID NO: 169 - DNA, 21 bp, Artificial
  siRNA antisense strand oligonucleotide
tgaagccatcaatcagtgctt

SEQ ID NO: 170 - DNA, 21 bp, Artificial
  siRNA antisense strand oligonucleotide
caggaaacaaatagaagcctt

SEQ ID NO: 171 - DNA, 21 bp, Artificial
  siRNA antisense strand oligonucleotide
gtcagatcaggattctgattt

SEQ ID NO: 172 - DNA, 21 bp, Artificial
  siRNA antisense strand oligonucleotide
taagccagtcagatcaggatt

SEQ ID NO: 173 - DNA, 21 bp, Artificial
  siRNA antisense strand oligonucleotide
tatggtcttggggagttggtt

SEQ ID NO: 174 - DNA, 21 bp, Artificial
  siRNA antisense strand oligonucleotide
ttgatatggtcttggggagtt

SEQ ID NO: 175 - DNA, 21 bp, Artificial
  siRNA antisense strand oligonucleotide
gggtcactttgatatggtctt

SEQ ID NO: 176 - DNA, 21 bp, Artificial
  siRNA antisense strand oligonucleotide
atattgttcctgggtcacttt

SEQ ID NO: 177 - DNA, 21 bp, Artificial
  siRNA antisense strand oligonucleotide
cacagtataattcatattgtt

SEQ ID NO: 178 - DNA, 21 bp, Artificial
  siRNA antisense strand oligonucleotide
tctcacagtataattcatatt

SEQ ID NO: 179 - DNA, 21 bp, Artificial
  siRNA antisense strand oligonucleotide
agcccatctcacagtataatt

SEQ ID NO: 180 - DNA, 21 bp, Artificial
  siRNA antisense strand oligonucleotide
aatctttacatccttatcatt

SEQ ID NO: 181 - DNA, 21 bp, Artificial
  siRNA antisense strand oligonucleotide
gggctcaatctttacatcctt

SEQ ID NO: 182 - DNA, 21 bp, Artificial
  siRNA antisense strand oligonucleotide
tgtccacagggctcaatcttt

SEQ ID NO: 183 - DNA, 21 bp, Artificial
  siRNA antisense strand oligonucleotide
gacagccctgaccttctgatt

SEQ ID NO: 184 - DNA, 21 bp, Artificial
  siRNA antisense strand oligonucleotide
agaaaggacagccctgacctt

SEQ ID NO: 185 - DNA, 21 bp, Artificial
  siRNA antisense strand oligonucleotide
tgggttcagtacctttaattt

SEQ ID NO: 186 - DNA, 21 bp, Artificial
  siRNA antisense strand oligonucleotide
cacgatgggttcagtaccttt

SEQ ID NO: 187 - DNA, 21 bp, Artificial
  siRNA antisense strand oligonucleotide
acggatctaccacgatgggtt

SEQ ID NO: 188 - DNA, 21 bp, Artificial
  siRNA antisense strand oligonucleotide
tcatcatcatcatcataattt

SEQ ID NO: 189 - DNA, 21 bp, Artificial
  siRNA antisense strand oligonucleotide
agagagtatcatcagctcgtt

SEQ ID NO: 190 - DNA, 21 bp, Artificial
  siRNA antisense strand oligonucleotide
cttggcaccagccaattcctt

SEQ ID NO: 191 - DNA, 21 bp, Artificial
  siRNA antisense strand oligonucleotide
ccaccttggcaccagccaatt

SEQ ID NO: 192 - DNA, 21 bp, Artificial
  siRNA antisense strand oligonucleotide
agaatggagaaggcggccgtt

SEQ ID NO: 193 - DNA, 21 bp, Artificial
  siRNA antisense strand oligonucleotide
gttccaagagcagaagcactt

SEQ ID NO: 194 - DNA, 21 bp, Artificial
  siRNA antisense strand oligonucleotide
gaagcagccttagaagcagtt

SEQ ID NO: 195 - DNA, 21 bp, Artificial
  siRNA antisense strand oligonucleotide
aagggagccagaagcagcctt

SEQ ID NO: 196 - DNA, 21 bp, Artificial
  siRNA antisense strand oligonucleotide
tactggcaatggtttgtcttt

SEQ ID NO: 197 - DNA, 21 bp, Artificial
  siRNA antisense strand oligonucleotide
gggaggtactggcaatggttt

SEQ ID NO: 198 - DNA, 21 bp, Artificial
  siRNA antisense strand oligonucleotide
ggcgtctttgaggtcgggatt

SEQ ID NO: 199 - DNA, 21 bp, Artificial
  siRNA antisense strand oligonucleotide
tacaaggcaaggggcgtcttt

SEQ ID NO: 200 - DNA, 21 bp, Artificial
  siRNA antisense strand oligonucleotide
gatactggtactttggggatt

SEQ ID NO: 201 - DNA, 21 bp, Artificial
  siRNA antisense strand oligonucleotide
tggggcagatactggtacttt

SEQ ID NO: 202 - DNA, 21 bp, Artificial
  siRNA antisense strand oligonucleotide
gtccagggatcactggaactt

SEQ ID NO: 203 - DNA, 21 bp, Artificial
  siRNA antisense strand oligonucleotide
tgccggttggttaattctctt

SEQ ID NO: 204 - DNA, 21 bp, Artificial
  siRNA antisense strand oligonucleotide
gtgagtgccggttggttaatt

SEQ ID NO: 205 - DNA, 21 bp, Artificial
  siRNA antisense strand oligonucleotide
ggaagtgagtgccggttggtt

SEQ ID NO: 206 - DNA, 21 bp, Artificial
  siRNA antisense strand oligonucleotide
aaatggaagtgagtgccggtt

SEQ ID NO: 207 - DNA, 21 bp, Artificial
  siRNA antisense strand oligonucleotide
catctggtctgggctccattt

SEQ ID NO: 208 - DNA, 21 bp, Artificial
  siRNA antisense strand oligonucleotide
gtatccagactgaacgtgctt

SEQ ID NO: 209 - DNA, 21 bp, Artificial
  siRNA antisense strand oligonucleotide
acctactaatgggctgctatt

SEQ ID NO: 210 - DNA, 21 bp, Artificial
  siRNA antisense strand oligonucleotide
gcagatgaggaaggtttgatt

SEQ ID NO: 211 - DNA, 21 bp, Artificial
  siRNA antisense strand oligonucleotide
attggcagatgaggaaggttt

SEQ ID NO: 212 - DNA, 21 bp, Artificial
  siRNA antisense strand oligonucleotide
agccagagaataaatggcatt

SEQ ID NO: 213 - DNA, 21 bp, Artificial
  siRNA antisense strand oligonucleotide
attgctccccaggtggcagtt

SEQ ID NO: 214 - DNA, 21 bp, Artificial
  siRNA antisense strand oligonucleotide
tgtcctcttcaccctcacatt

SEQ ID NO: 215 - DNA, 21 bp, Artificial
  siRNA antisense strand oligonucleotide
tcatgtactctgtgtcctctt

SEQ ID NO: 216 - DNA, 21 bp, Artificial
  siRNA antisense strand oligonucleotide
actgaatattatacattgctt

SEQ ID NO: 217 - DNA, 21 bp, Artificial
  siRNA antisense strand oligonucleotide
tgggactgaatattatacatt

SEQ ID NO: 218 - DNA, 21 bp, Artificial
  siRNA antisense strand oligonucleotide
tggcgcctgggactgaatatt

SEQ ID NO: 219 - DNA, 21 bp, Artificial
  siRNA antisense strand oligonucleotide
tgactcctcgggaccagtgtt

SEQ ID NO: 220 - DNA, 21 bp, Artificial
  siRNA antisense strand oligonucleotide
atcatacccatcatcctcatt

SEQ ID NO: 221 - DNA, 21 bp, Artificial
  siRNA antisense strand oligonucleotide
ttagagatatctgagagagtt

SEQ ID NO: 222 - DNA, 21 bp, Artificial
  siRNA antisense strand oligonucleotide
gccaaaggaggagctggcatt

SEQ ID NO: 223 - DNA, 21 bp, Artificial
  siRNA antisense strand oligonucleotide
gaaccttcagtgacatttgtt

SEQ ID NO: 224 - DNA, 21 bp, Artificial
  siRNA antisense strand oligonucleotide
tgggaaccttcagtgacattt

SEQ ID NO: 225 - DNA, 21 bp, Artificial
  siRNA antisense strand oligonucleotide
tctcgggaacttgggaacctt

SEQ ID NO: 226 - DNA, 21 bp, Artificial
  siRNA antisense strand oligonucleotide
ttggaggcctctcgggaactt

SEQ ID NO: 227 - DNA, 21 bp, Artificial
  siRNA antisense strand oligonucleotide
tgattctccgcgggaatggtt

SEQ ID NO: 228 - DNA, 21 bp, Artificial
  siRNA antisense strand oligonucleotide
gctttccgttcagagttgatt

SEQ ID NO: 229 - DNA, 21 bp, Artificial
  siRNA antisense strand oligonucleotide
gccagctttccgttcagagtt

SEQ ID NO: 230 - DNA, 21 bp, Artificial
  siRNA antisense strand oligonucleotide
gacagctgccagctttccgtt

SEQ ID NO: 231 - DNA, 21 bp, Artificial
  siRNA antisense strand oligonucleotide
ttgctgacagctgccagcttt

SEQ ID NO: 232 - DNA, 21 bp, Artificial
  siRNA antisense strand oligonucleotide
gtacccctgactcatgaggtt

SEQ ID NO: 233 - DNA, 21 bp, Artificial
  siRNA antisense strand oligonucleotide
ctgggcaatgaccaaagcttt

SEQ ID NO: 234 - DNA, 21 bp, Artificial
  siRNA antisense strand oligonucleotide
tttggccatctcgatgttgtt

SEQ ID NO: 235 - DNA, 21 bp, Artificial
  siRNA antisense strand oligonucleotide
acaaattcccggaggatgttt

SEQ ID NO: 236 - DNA, 21 bp, Artificial
  siRNA antisense strand oligonucleotide
gagaagaaatggaaacaaatt

SEQ ID NO: 237 - DNA, 21 bp, Artificial
  siRNA antisense strand oligonucleotide
ctgcccttctaggtgccactt

SEQ ID NO: 238 - DNA, 21 bp, Artificial
  siRNA antisense strand oligonucleotide
accaaaggaactcctgccctt

SEQ ID NO: 239 - DNA, 21 bp, Artificial
  siRNA antisense strand oligonucleotide
cccacagagagggcaagactt

SEQ ID NO: 240 - DNA, 21 bp, Artificial
  siRNA antisense strand oligonucleotide
atttcaccactttgaaatctt

SEQ ID NO: 241 - DNA, 21 bp, Artificial
  siRNA antisense strand oligonucleotide
aaacatactagctgctccatt

SEQ ID NO: 242 - DNA, 3 bp, Artificial
  shRNA loop sequence
ccc

SEQ ID NO: 243 - DNA, 4 bp, Artificial
  shRNA loop sequence
ttcg

SEQ ID NO: 244 - DNA, 5 bp, Artificial
  shRNA loop sequence
ccacc

SEQ ID NO: 245 - DNA, 6 bp, Artificial
  shRNA loop sequence
ctcgag

SEQ ID NO: 246 - DNA, 6 bp, Artificial
  shRNA loop sequence
aagctt

SEQ ID NO: 247 - DNA, 7 bp, Artificial
  shRNA loop sequence
ccacacc

SEQ ID NO: 248 - DNA, 9 bp, Artificial
  shRNA loop sequence
ttcaagaga

SEQ ID NO: 249 - DNA, 2721 bp, Artificial
  c-Cbl G306E
  CDS (1)..(2718)
atggccggcaacgtgaagaagagctctggggccgggggcggcacgggctccgggggctcgggttcgggtggcctgattgggctcatgaaggacgccttccagccgcaccaccaccaccaccaccacctcagcccccacccgccggggacggtggacaagaagatggtggagaagtgctggaagctcatggacaaggtggtgcggttgtgtcagaacccaaagctggcgctaaagaatagcccaccttatatcttagacctgctaccagatacctaccagcatctccgtactatcttgtcaagatatgaggggaagatggagacacttggagaaaatgagtattttagggtgtttatggagaatttgatgaagaaaactaagcaaaccataagcctcttcaaggagggaaaagaaagaatgtatgaggagaattctcagcctaggcgaaacctaaccaaactgtccctcatcttcagccacatgctggcagaactaaaaggaatctttccaagtggactctttcagggagacacatttcggattactaaagcagatgctgcggaattttggagaaaagcttttggggaaaagacaatagtcccttggaagagctttcgacaggctctacatgaagtgcatcccatcagttctgggctggaggccatggctctgaaatccactattgatctgacctgcaatgattatatttcggtttttgaatttgacatctttacccgactctttcagccctggtcctctttgctcaggaattggaacagccttgctgtaactcatcctggctacatggcttttttgacgtatgacgaagtgaaagctcggctccagaaattcattcacaaacctggcagttatatcttccggctgagctgtactcgtctgggtcagtgggctattgagtatgttactgctgatgggaacattctccagacaatccctcacaataaacctctcttccaagcactgattgatggcttcagggaaggcttctatttgtttcctgatggacgaaatcagaatcctgatctgactggcttatgtgaaccaactccccaagaccatatcaaagtgacccaggaacaatatgaattatactgtgagatgggctccacattccaactatgtaaaatatgtgctgaaaatgataaggatgtaaagattgagccctgtggacacctcatgtgcacatcctgtcttacatcctggcaggaatcagaaggtcagggctgtcctttctgccgatgtgaaattaaaggtactgaacccatcgtggtagatccgtttgatcctagagggagtggcagcctgttgaggcaaggagcagagggagctccctccccaaattatgatgatgatgatgatgaacgagctgatgatactctcttcatgatgaaggaattggctggtgccaaggtggaacggccgccttctccattctccatggccccacaagcttcccttcccccggtgccaccacgacttgaccttctgccgcagcgagtatgtgttccctcaagtgcttctgctcttggaactgcttctaaggctgcttctggctcccttcataaagacaaaccattgccagtacctcccacacttcgagatcttccaccaccaccgcctccagaccggccatattctgttggagcagaatcccgacctcaaagacgccccttgccttgtacaccaggcgactgtccctccagagacaaactgccccctgtcccctctagccgccttggagactcatggctgccccggccaatccccaaagtaccagtatctgccccaagttccagtgatccctggacaggaagagaattaaccaaccggcactcacttccattttcattgccctcacaaatggagcccagaccagatgtgcctaggctcggaagcacgttcagtctggatacctccatgagtatgaatagcagcccattagtaggtccagagtgtgaccaccccaaaatcaaaccttcctcatctgccaatgccatttattctctggctgccagacctcttcctgtgccaaaactgccacctggggagcaatgtgagggtgaagaggacacagagtacatgactccctcttccaggcctctacggcctttggatacatcccagagttcacgagcatgtgattgcgaccagcagattgatagctgtacgtatgaagcaatgtataatattcagtcccaggcgccatctatcaccgagagcagcacctttggtgaagggaatttggccgcagcccatgccaacactggtcccgaggagtcagaaaatgaggatgatgggtatgatgtcccaaagccacctgtgccggccgtgctggcccgccgaactctctcagatatctctaatgccagctcctcctttggctggttgtctctggatggtgatcctacaacaaatgtcactgaaggttcccaagttcccgagaggcctccaaaaccattcccgcggagaatcaactctgaacggaaagctggcagctgtcagcaaggtagtggtcctgccgcctctgctgccaccgcctcacctcagctctccagtgagatcgagaacctcatgagtcaggggtactcctaccaggacatccagaaagctttggtcattgcccagaacaacatcgagatggccaaaaacatcctccgggaatttgtttccatttcttctcctgcccatgtagctacctag

SEQ ID NO: 250 - protein, 906 aa, Artificial
  Synthetic Construct
MAGNVKKSSGAGGGTGSGGSGSGGLIGLMKDAFQPHHHHHHHLSPHPPGTVDKKMVEKCWKLMDKVVRLCQNPKLALKNSPPYILDLLPDTYQHLRTILSRYEGKMETLGENEYFRVFMENLMKKTKQTISLFKEGKERMYEENSQPRRNLTKLSLIFSHMLAELKGIFPSGLFQGDTFRITKADAAEFWRKAFGEKTIVPWKSFRQALHEVHPISSGLEAMALKSTIDLTCNDYISVFEFDIFTRLFQPWSSLLRNWNSLAVTHPGYMAFLTYDEVKARLQKFIHKPGSYIFRLSCTRLGQWAIEYVTADGNILQTIPHNKPLFQALIDGFREGFYLFPDGRNQNPDLTGLCEPTPQDHIKVTQEQYELYCEMGSTFQLCKICAENDKDVKIEPCGHLMCTSCLTSWQESEGQGCPFCRCEIKGTEPIVVDPFDPRGSGSLLRQGAEGAPSPNYDDDDDERADDTLFMMKELAGAKVERPPSPFSMAPQASLPPVPPRLDLLPQRVCVPSSASALGTASKAASGSLHKDKPLPVPPTLRDLPPPPPPDRPYSVGAESRPQRRPLPCTPGDCPSRDKLPPVPSSRLGDSWLPRPIPKVPVSAPSSSDPWTGRELTNRHSLPFSLPSQMEPRPDVPRLGSTFSLDTSMSMNSSPLVGPECDHPKIKPSSSANAIYSLAARPLPVPKLPPGEQCEGEEDTEYMTPSSRPLRPLDTSQSSRACDCDQQIDSCTYEAMYNIQSQAPSITESSTFGEGNLAAAHANTGPEESENEDDGYDVPKPPVPAVLARRTLSDISNASSSFGWLSLDGDPTTNVTEGSQVPERPPKPFPRRINSERKAGSCQQGSGPAASAATASPQLSSEIENLMSQGYSYQDIQKALVIAQNNIEMAKNILREFVSISSPAHVAT

SEQ ID NO: 251 - DNA, 2721 bp, Artificial
  c-Cbl C381A
  CDS (1)..(2718)
atggccggcaacgtgaagaagagctctggggccgggggcggcacgggctccgggggctcgggttcgggtggcctgattgggctcatgaaggacgccttccagccgcaccaccaccaccaccaccacctcagcccccacccgccggggacggtggacaagaagatggtggagaagtgctggaagctcatggacaaggtggtgcggttgtgtcagaacccaaagctggcgctaaagaatagcccaccttatatcttagacctgctaccagatacctaccagcatctccgtactatcttgtcaagatatgaggggaagatggagacacttggagaaaatgagtattttagggtgtttatggagaatttgatgaagaaaactaagcaaaccataagcctcttcaaggagggaaaagaaagaatgtatgaggagaattctcagcctaggcgaaacctaaccaaactgtccctcatcttcagccacatgctggcagaactaaaaggaatctttccaagtggactctttcagggagacacatttcggattactaaagcagatgctgcggaattttggagaaaagcttttggggaaaagacaatagtcccttggaagagctttcgacaggctctacatgaagtgcatcccatcagttctgggctggaggccatggctctgaaatccactattgatctgacctgcaatgattatatttcggtttttgaatttgacatctttacccgactctttcagccctggtcctctttgctcaggaattggaacagccttgctgtaactcatcctggctacatggcttttttgacgtatgacgaagtgaaagctcggctccagaaattcattcacaaacctggcagttatatcttccggctgagctgtactcgtctgggtcagtgggctattgggtatgttactgctgatgggaacattctccagacaatccctcacaataaacctctcttccaagcactgattgatggcttcagggaaggcttctatttgtttcctgatggacgaaatcagaatcctgatctgactggcttatgtgaaccaactccccaagaccatatcaaagtgacccaggaacaatatgaattatactgtgagatgggctccacattccaactagctaaaatatgtgctgaaaatgataaggatgtaaagattgagccctgtggacacctcatgtgcacatcctgtcttacatcctggcaggaatcagaaggtcagggctgtcctttctgccgatgtgaaattaaaggtactgaacccatcgtggtagatccgtttgatcctagagggagtggcagcctgttgaggcaaggagcagagggagctccctccccaaattatgatgatgatgatgatgaacgagctgatgatactctcttcatgatgaaggaattggctggtgccaaggtggaacggccgccttctccattctccatggccccacaagcttcccttcccccggtgccaccacgacttgaccttctgccgcagcgagtatgtgttccctcaagtgcttctgctcttggaactgcttctaaggctgcttctggctcccttcataaagacaaaccattgccagtacctcccacacttcgagatcttccaccaccaccgcctccagaccggccatattctgttggagcagaatcccgacctcaaagacgccccttgccttgtacaccaggcgactgtccctccagagacaaactgccccctgtcccctctagccgccttggagactcatggctgccccggccaatccccaaagtaccagtatctgccccaagttccagtgatccctggacaggaagagaattaaccaaccggcactcacttccattttcattgccctcacaaatggagcccagaccagatgtgcctaggctcggaagcacgttcagtctggatacctccatgagtatgaatagcagcccattagtaggtccagagtgtgaccaccccaaaatcaaaccttcctcatctgccaatgccatttattctctggctgccagacctcttcctgtgccaaaactgccacctggggagcaatgtgagggtgaagaggacacagagtacatgactccctcttccaggcctctacggcctttggatacatcccagagttcacgagcatgtgattgcgaccagcagattgatagctgtacgtatgaagcaatgtataatattcagtcccaggcgccatctatcaccgagagcagcacctttggtgaagggaatttggccgcagcccatgccaacactggtcccgaggagtcagaaaatgaggatgatgggtatgatgtcccaaagccacctgtgccggccgtgctggcccgccgaactctctcagatatctctaatgccagctcctcctttggctggttgtctctggatggtgatcctacaacaaatgtcactgaaggttcccaagttcccgagaggcctccaaaaccattcccgcggagaatcaactctgaacggaaagctggcagctgtcagcaaggtagtggtcctgccgcctctgctgccaccgcctcacctcagctctccagtgagatcgagaacctcatgagtcaggggtactcctaccaggacatccagaaagctttggtcattgcccagaacaacatcgagatggccaaaaacatcctccgggaatttgtttccatttcttctcctgcccatgtagctacctag

SEQ ID NO: 252 - protein, 906 aa, Artificial
  Synthetic Construct
MAGNVKKSSGAGGGTGSGGSGSGGLIGLMKDAFQPHHHHHHHLSPHPPGTVDKKMVEKCWKLMDKVVRLCQNPKLALKNSPPYILDLLPDTYQHLRTILSRYEGKMETLGENEYFRVFMENLMKKTKQTISLFKEGKERMYEENSQPRRNLTKLSLIFSHMLAELKGIFPSGLFQGDTFRITKADAAEFWRKAFGEKTIVPWKSFRQALHEVHPISSGLEAMALKSTIDLTCNDYISVFEFDIFTRLFQPWSSLLRNWNSLAVTHPGYMAFLTYDEVKARLQKFIHKPGSYIFRLSCTRLGQWAIGYVTADGNILQTIPHNKPLFQALIDGFREGFYLFPDGRNQNPDLTGLCEPTPQDHIKVTQEQYELYCEMGSTFQLAKICAENDKDVKIEPCGHLMCTSCLTSWQESEGQGCPFCRCEIKGTEPIVVDPFDPRGSGSLLRQGAEGAPSPNYDDDDDERADDTLFMMKELAGAKVERPPSPFSMAPQASLPPVPPRLDLLPQRVCVPSSASALGTASKAASGSLHKDKPLPVPPTLRDLPPPPPPDRPYSVGAESRPQRRPLPCTPGDCPSRDKLPPVPSSRLGDSWLPRPIPKVPVSAPSSSDPWTGRELTNRHSLPFSLPSQMEPRPDVPRLGSTFSLDTSMSMNSSPLVGPECDHPKIKPSSSANAIYSLAARPLPVPKLPPGEQCEGEEDTEYMTPSSRPLRPLDTSQSSRACDCDQQIDSCTYEAMYNIQSQAPSITESSTFGEGNLAAAHANTGPEESENEDDGYDVPKPPVPAVLARRTLSDISNASSSFGWLSLDGDPTTNVTEGSQVPERPPKPFPRRINSERKAGSCQQGSGPAASAATASPQLSSEIENLMSQGYSYQDIQKALVIAQNNIEMAKNILREFVSISSPAHVAT

SEQ ID NO: 253 - DNA, 2721 bp, Artificial
  c-Cbl Y700F
  CDS (1)..(2718)
atggccggcaacgtgaagaagagctctggggccgggggcggcacgggctccgggggctcgggttcgggtggcctgattgggctcatgaaggacgccttccagccgcaccaccaccaccaccaccacctcagcccccacccgccggggacggtggacaagaagatggtggagaagtgctggaagctcatggacaaggtggtgcggttgtgtcagaacccaaagctggcgctaaagaatagcccaccttatatcttagacctgctaccagatacctaccagcatctccgtactatcttgtcaagatatgaggggaagatggagacacttggagaaaatgagtattttagggtgtttatggagaatttgatgaagaaaactaagcaaaccataagcctcttcaaggagggaaaagaaagaatgtatgaggagaattctcagcctaggcgaaacctaaccaaactgtccctcatcttcagccacatgctggcagaactaaaaggaatctttccaagtggactctttcagggagacacatttcggattactaaagcagatgctgcggaattttggagaaaagcttttggggaaaagacaatagtcccttggaagagctttcgacaggctctacatgaagtgcatcccatcagttctgggctggaggccatggctctgaaatccactattgatctgacctgcaatgattatatttcggtttttgaatttgacatctttacccgactctttcagccctggtcctctttgctcaggaattggaacagccttgctgtaactcatcctggctacatggcttttttgacgtatgacgaagtgaaagctcggctccagaaattcattcacaaacctggcagttatatcttccggctgagctgtactcgtctgggtcagtgggctattgggtatgttactgctgatgggaacattctccagacaatccctcacaataaacctctcttccaagcactgattgatggcttcagggaaggcttctatttgtttcctgatggacgaaatcagaatcctgatctgactggcttatgtgaaccaactccccaagaccatatcaaagtgacccaggaacaatatgaattatactgtgagatgggctccacattccaactatgtaaaatatgtgctgaaaatgataaggatgtaaagattgagccctgtggacacctcatgtgcacatcctgtcttacatcctggcaggaatcagaaggtcagggctgtcctttctgccgatgtgaaattaaaggtactgaacccatcgtggtagatccgtttgatcctagagggagtggcagcctgttgaggcaaggagcagagggagctccctccccaaattatgatgatgatgatgatgaacgagctgatgatactctcttcatgatgaaggaattggctggtgccaaggtggaacggccgccttctccattctccatggccccacaagcttcccttcccccggtgccaccacgacttgaccttctgccgcagcgagtatgtgttccctcaagtgcttctgctcttggaactgcttctaaggctgcttctggctcccttcataaagacaaaccattgccagtacctcccacacttcgagatcttccaccaccaccgcctccagaccggccatattctgttggagcagaatcccgacctcaaagacgccccttgccttgtacaccaggcgactgtccctccagagacaaactgccccctgtcccctctagccgccttggagactcatggctgccccggccaatccccaaagtaccagtatctgccccaagttccagtgatccctggacaggaagagaattaaccaaccggcactcacttccattttcattgccctcacaaatggagcccagaccagatgtgcctaggctcggaagcacgttcagtctggatacctccatgagtatgaatagcagcccattagtaggtccagagtgtgaccaccccaaaatcaaaccttcctcatctgccaatgccatttattctctggctgccagacctcttcctgtgccaaaactgccacctggggagcaatgtgagggtgaagaggacacagagttcatgactccctcttccaggcctctacggcctttggatacatcccagagttcacgagcatgtgattgcgaccagcagattgatagctgtacgtatgaagcaatgtataatattcagtcccaggcgccatctatcaccgagagcagcacctttggtgaagggaatttggccgcagcccatgccaacactggtcccgaggagtcagaaaatgaggatgatgggtatgatgtcccaaagccacctgtgccggccgtgctggcccgccgaactctctcagatatctctaatgccagctcctcctttggctggttgtctctggatggtgatcctacaacaaatgtcactgaaggttcccaagttcccgagaggcctccaaaaccattcccgcggagaatcaactctgaacggaaagctggcagctgtcagcaaggtagtggtcctgccgcctctgctgccaccgcctcacctcagctctccagtgagatcgagaacctcatgagtcaggggtactcctaccaggacatccagaaagctttggtcattgcccagaacaacatcgagatggccaaaaacatcctccgggaatttgtttccatttcttctcctgcccatgtagctacctag

SEQ ID NO: 254 - protein, 906 aa, Artificial
  Synthetic Construct
MAGNVKKSSGAGGGTGSGGSGSGGLIGLMKDAFQPHHHHHHHLSPHPPGTVDKKMVEKCWKLMDKVVRLCQNPKLALKNSPPYILDLLPDTYQHLRTILSRYEGKMETLGENEYFRVFMENLMKKTKQTISLFKEGKERMYEENSQPRRNLTKLSLIFSHMLAELKGIFPSGLFQGDTFRITKADAAEFWRKAFGEKTIVPWKSFRQALHEVHPISSGLEAMALKSTIDLTCNDYISVFEFDIFTRLFQPWSSLLRNWNSLAVTHPGYMAFLTYDEVKARLQKFIHKPGSYIFRLSCTRLGQWAIGYVTADGNILQTIPHNKPLFQALIDGFREGFYLFPDGRNQNPDLTGLCEPTPQDHIKVTQEQYELYCEMGSTFQLCKICAENDKDVKIEPCGHLMCTSCLTSWQESEGQGCPFCRCEIKGTEPIVVDPFDPRGSGSLLRQGAEGAPSPNYDDDDDERADDTLFMMKELAGAKVERPPSPFSMAPQASLPPVPPRLDLLPQRVCVPSSASALGTASKAASGSLHKDKPLPVPPTLRDLPPPPPPDRPYSVGAESRPQRRPLPCTPGDCPSRDKLPPVPSSRLGDSWLPRPIPKVPVSAPSSSDPWTGRELTNRHSLPFSLPSQMEPRPDVPRLGSTFSLDTSMSMNSSPLVGPECDHPKIKPSSSANAIYSLAARPLPVPKLPPGEQCEGEEDTEFMTPSSRPLRPLDTSQSSRACDCDQQIDSCTYEAMYNIQSQAPSITESSTFGEGNLAAAHANTGPEESENEDDGYDVPKPPVPAVLARRTLSDISNASSSFGWLSLDGDPTTNVTEGSQVPERPPKPFPRRINSERKAGSCQQGSGPAASAATASPQLSSEIENLMSQGYSYQDIQKALVIAQNNIEMAKNILREFVSISSPAHVAT

SEQ ID NO: 255 - DNA, 2721 bp, Artificial
  c-Cbl Y731F
  CDS (1)..(2718)
atggccggcaacgtgaagaagagctctggggccgggggcggcacgggctccgggggctcgggttcgggtggcctgattgggctcatgaaggacgccttccagccgcaccaccaccaccaccaccacctcagcccccacccgccggggacggtggacaagaagatggtggagaagtgctggaagctcatggacaaggtggtgcggttgtgtcagaacccaaagctggcgctaaagaatagcccaccttatatcttagacctgctaccagatacctaccagcatctccgtactatcttgtcaagatatgaggggaagatggagacacttggagaaaatgagtattttagggtgtttatggagaatttgatgaagaaaactaagcaaaccataagcctcttcaaggagggaaaagaaagaatgtatgaggagaattctcagcctaggcgaaacctaaccaaactgtccctcatcttcagccacatgctggcagaactaaaaggaatctttccaagtggactctttcagggagacacatttcggattactaaagcagatgctgcggaattttggagaaaagcttttggggaaaagacaatagtcccttggaagagctttcgacaggctctacatgaagtgcatcccatcagttctgggctggaggccatggctctgaaatccactattgatctgacctgcaatgattatatttcggtttttgaatttgacatctttacccgactctttcagccctggtcctctttgctcaggaattggaacagccttgctgtaactcatcctggctacatggcttttttgacgtatgacgaagtgaaagctcggctccagaaattcattcacaaacctggcagttatatcttccggctgagctgtactcgtctgggtcagtgggctattgggtatgttactgctgatgggaacattctccagacaatccctcacaataaacctctcttccaagcactgattgatggcttcagggaaggcttctatttgtttcctgatggacgaaatcagaatcctgatctgactggcttatgtgaaccaactccccaagaccatatcaaagtgacccaggaacaatatgaattatactgtgagatgggctccacattccaactatgtaaaatatgtgctgaaaatgataaggatgtaaagattgagccctgtggacacctcatgtgcacatcctgtcttacatcctggcaggaatcagaaggtcagggctgtcctttctgccgatgtgaaattaaaggtactgaacccatcgtggtagatccgtttgatcctagagggagtggcagcctgttgaggcaaggagcagagggagctccctccccaaattatgatgatgatgatgatgaacgagctgatgatactctcttcatgatgaaggaattggctggtgccaaggtggaacggccgccttctccattctccatggccccacaagcttcccttcccccggtgccaccacgacttgaccttctgccgcagcgagtatgtgttccctcaagtgcttctgctcttggaactgcttctaaggctgcttctggctcccttcataaagacaaaccattgccagtacctcccacacttcgagatcttccaccaccaccgcctccagaccggccatattctgttggagcagaatcccgacctcaaagacgccccttgccttgtacaccaggcgactgtccctccagagacaaactgccccctgtcccctctagccgccttggagactcatggctgccccggccaatccccaaagtaccagtatctgccccaagttccagtgatccctggacaggaagagaattaaccaaccggcactcacttccattttcattgccctcacaaatggagcccagaccagatgtgcctaggctcggaagcacgttcagtctggatacctccatgagtatgaatagcagcccattagtaggtccagagtgtgaccaccccaaaatcaaaccttcctcatctgccaatgccatttattctctggctgccagacctcttcctgtgccaaaactgccacctggggagcaatgtgagggtgaagaggacacagagtacatgactccctcttccaggcctctacggcctttggatacatcccagagttcacgagcatgtgattgcgaccagcagattgatagctgtacgtttgaagcaatgtataatattcagtcccaggcgccatctatcaccgagagcagcacctttggtgaagggaatttggccgcagcccatgccaacactggtcccgaggagtcagaaaatgaggatgatgggtatgatgtcccaaagccacctgtgccggccgtgctggcccgccgaactctctcagatatctctaatgccagctcctcctttggctggttgtctctggatggtgatcctacaacaaatgtcactgaaggttcccaagttcccgagaggcctccaaaaccattcccgcggagaatcaactctgaacggaaagctggcagctgtcagcaaggtagtggtcctgccgcctctgctgccaccgcctcacctcagctctccagtgagatcgagaacctcatgagtcaggggtactcctaccaggacatccagaaagctttggtcattgcccagaacaacatcgagatggccaaaaacatcctccgggaatttgtttccatttcttctcctgcccatgtagctacctag

SEQ ID NO: 256 - protein, 906 aa, Artificial
  Synthetic Construct
MAGNVKKSSGAGGGTGSGGSGSGGLIGLMKDAFQPHHHHHHHLSPHPPGTVDKKMVEKCWKLMDKVVRLCQNPKLALKNSPPYILDLLPDTYQHLRTILSRYEGKMETLGENEYFRVFMENLMKKTKQTISLFKEGKERMYEENSQPRRNLTKLSLIFSHMLAELKGIFPSGLFQGDTFRITKADAAEFWRKAFGEKTIVPWKSFRQALHEVHPISSGLEAMALKSTIDLTCNDYISVFEFDIFTRLFQPWSSLLRNWNSLAVTHPGYMAFLTYDEVKARLQKFIHKPGSYIFRLSCTRLGQWAIGYVTADGNILQTIPHNKPLFQALIDGFREGFYLFPDGRNQNPDLTGLCEPTPQDHIKVTQEQYELYCEMGSTFQLCKICAENDKDVKIEPCGHLMCTSCLTSWQESEGQGCPFCRCEIKGTEPIVVDPFDPRGSGSLLRQGAEGAPSPNYDDDDDERADDTLFMMKELAGAKVERPPSPFSMAPQASLPPVPPRLDLLPQRVCVPSSASALGTASKAASGSLHKDKPLPVPPTLRDLPPPPPPDRPYSVGAESRPQRRPLPCTPGDCPSRDKLPPVPSSRLGDSWLPRPIPKVPVSAPSSSDPWTGRELTNRHSLPFSLPSQMEPRPDVPRLGSTFSLDTSMSMNSSPLVGPECDHPKIKPSSSANAIYSLAARPLPVPKLPPGEQCEGEEDTEYMTPSSRPLRPLDTSQSSRACDCDQQIDSCTFEAMYNIQSQAPSITESSTFGEGNLAAAHANTGPEESENEDDGYDVPKPPVPAVLARRTLSDISNASSSFGWLSLDGDPTTNVTEGSQVPERPPKPFPRRINSERKAGSCQQGSGPAASAATASPQLSSEIENLMSQGYSYQDIQKALVIAQNNIEMAKNILREFVSISSPAHVAT

SEQ ID NO: 257 - DNA, 2721 bp, Artificial
  c-Cbl Y774F
  CDS (1)..(2718)
atggccggcaacgtgaagaagagctctggggccgggggcggcacgggctccgggggctcgggttcgggtggcctgattgggctcatgaaggacgccttccagccgcaccaccaccaccaccaccacctcagcccccacccgccggggacggtggacaagaagatggtggagaagtgctggaagctcatggacaaggtggtgcggttgtgtcagaacccaaagctggcgctaaagaatagcccaccttatatcttagacctgctaccagatacctaccagcatctccgtactatcttgtcaagatatgaggggaagatggagacacttggagaaaatgagtattttagggtgtttatggagaatttgatgaagaaaactaagcaaaccataagcctcttcaaggagggaaaagaaagaatgtatgaggagaattctcagcctaggcgaaacctaaccaaactgtccctcatcttcagccacatgctggcagaactaaaaggaatctttccaagtggactctttcagggagacacatttcggattactaaagcagatgctgcggaattttggagaaaagcttttggggaaaagacaatagtcccttggaagagctttcgacaggctctacatgaagtgcatcccatcagttctgggctggaggccatggctctgaaatccactattgatctgacctgcaatgattatatttcggtttttgaatttgacatctttacccgactctttcagccctggtcctctttgctcaggaattggaacagccttgctgtaactcatcctggctacatggcttttttgacgtatgacgaagtgaaagctcggctccagaaattcattcacaaacctggcagttatatcttccggctgagctgtactcgtctgggtcagtgggctattgggtatgttactgctgatgggaacattctccagacaatccctcacaataaacctctcttccaagcactgattgatggcttcagggaaggcttctatttgtttcctgatggacgaaatcagaatcctgatctgactggcttatgtgaaccaactccccaagaccatatcaaagtgacccaggaacaatatgaattatactgtgagatgggctccacattccaactatgtaaaatatgtgctgaaaatgataaggatgtaaagattgagccctgtggacacctcatgtgcacatcctgtcttacatcctggcaggaatcagaaggtcagggctgtcctttctgccgatgtgaaattaaaggtactgaacccatcgtggtagatccgtttgatcctagagggagtggcagcctgttgaggcaaggagcagagggagctccctccccaaattatgatgatgatgatgatgaacgagctgatgatactctcttcatgatgaaggaattggctggtgccaaggtggaacggccgccttctccattctccatggccccacaagcttcccttcccccggtgccaccacgacttgaccttctgccgcagcgagtatgtgttccctcaagtgcttctgctcttggaactgcttctaaggctgcttctggctcccttcataaagacaaaccattgccagtacctcccacacttcgagatcttccaccaccaccgcctccagaccggccatattctgttggagcagaatcccgacctcaaagacgccccttgccttgtacaccaggcgactgtccctccagagacaaactgccccctgtcccctctagccgccttggagactcatggctgccccggccaatccccaaagtaccagtatctgccccaagttccagtgatccctggacaggaagagaattaaccaaccggcactcacttccattttcattgccctcacaaatggagcccagaccagatgtgcctaggctcggaagcacgttcagtctggatacctccatgagtatgaatagcagcccattagtaggtccagagtgtgaccaccccaaaatcaaaccttcctcatctgccaatgccatttattctctggctgccagacctcttcctgtgccaaaactgccacctggggagcaatgtgagggtgaagaggacacagagtacatgactccctcttccaggcctctacggcctttggatacatcccagagttcacgagcatgtgattgcgaccagcagattgatagctgtacgtatgaagcaatgtataatattcagtcccaggcgccatctatcaccgagagcagcacctttggtgaagggaatttggccgcagcccatgccaacactggtcccgaggagtcagaaaatgaggatgatgggtttgatgtcccaaagccacctgtgccggccgtgctggcccgccgaactctctcagatatctctaatgccagctcctcctttggctggttgtctctggatggtgatcctacaacaaatgtcactgaaggttcccaagttcccgagaggcctccaaaaccattcccgcggagaatcaactctgaacggaaagctggcagctgtcagcaaggtagtggtcctgccgcctctgctgccaccgcctcacctcagctctccagtgagatcgagaacctcatgagtcaggggtactcctaccaggacatccagaaagctttggtcattgcccagaacaacatcgagatggccaaaaacatcctccgggaatttgtttccatttcttctcctgcccatgtagctacctag

SEQ ID NO: 258 - protein, 906 aa, Artificial
  Synthetic Construct
MAGNVKKSSGAGGGTGSGGSGSGGLIGLMKDAFQPHHHHHHHLSPHPPGTVDKKMVEKCWKLMDKVVRLCQNPKLALKNSPPYILDLLPDTYQHLRTILSRYEGKMETLGENEYFRVFMENLMKKTKQTISLFKEGKERMYEENSQPRRNLTKLSLIFSHMLAELKGIFPSGLFQGDTFRITKADAAEFWRKAFGEKTIVPWKSFRQALHEVHPISSGLEAMALKSTIDLTCNDYISVFEFDIFTRLFQPWSSLLRNWNSLAVTHPGYMAFLTYDEVKARLQKFIHKPGSYIFRLSCTRLGQWAIGYVTADGNILQTIPHNKPLFQALIDGFREGFYLFPDGRNQNPDLTGLCEPTPQDHIKVTQEQYELYCEMGSTFQLCKICAENDKDVKIEPCGHLMCTSCLTSWQESEGQGCPFCRCEIKGTEPIVVDPFDPRGSGSLLRQGAEGAPSPNYDDDDDERADDTLFMMKELAGAKVERPPSPFSMAPQASLPPVPPRLDLLPQRVCVPSSASALGTASKAASGSLHKDKPLPVPPTLRDLPPPPPPDRPYSVGAESRPQRRPLPCTPGDCPSRDKLPPVPSSRLGDSWLPRPIPKVPVSAPSSSDPWTGRELTNRHSLPFSLPSQMEPRPDVPRLGSTFSLDTSMSMNSSPLVGPECDHPKIKPSSSANAIYSLAARPLPVPKLPPGEQCEGEEDTEYMTPSSRPLRPLDTSQSSRACDCDQQIDSCTYEAMYNIQSQAPSITESSTFGEGNLAAAHANTGPEESENEDDGFDVPKPPVPAVLARRTLSDISNASSSFGWLSLDGDPTTNVTEGSQVPERPPKPFPRRINSERKAGSCQQGSGPAASAATASPQLSSEIENLMSQGYSYQDIQKALVIAQNNIEMAKNILREFVSISSPAHVAT

SEQ ID NO: 259 - DNA, 2721 bp, Artificial
  c-Cbl Y700F/Y731F/Y774F
  CDS (1)..(2718)
atggccggcaacgtgaagaagagctctggggccgggggcggcacgggctccgggggctcgggttcgggtggcctgattgggctcatgaaggacgccttccagccgcaccaccaccaccaccaccacctcagcccccacccgccggggacggtggacaagaagatggtggagaagtgctggaagctcatggacaaggtggtgcggttgtgtcagaacccaaagctggcgctaaagaatagcccaccttatatcttagacctgctaccagatacctaccagcatctccgtactatcttgtcaagatatgaggggaagatggagacacttggagaaaatgagtattttagggtgtttatggagaatttgatgaagaaaactaagcaaaccataagcctcttcaaggagggaaaagaaagaatgtatgaggagaattctcagcctaggcgaaacctaaccaaactgtccctcatcttcagccacatgctggcagaactaaaaggaatctttccaagtggactctttcagggagacacatttcggattactaaagcagatgctgcggaattttggagaaaagcttttggggaaaagacaatagtcccttggaagagctttcgacaggctctacatgaagtgcatcccatcagttctgggctggaggccatggctctgaaatccactattgatctgacctgcaatgattatatttcggtttttgaatttgacatctttacccgactctttcagccctggtcctctttgctcaggaattggaacagccttgctgtaactcatcctggctacatggcttttttgacgtatgacgaagtgaaagctcggctccagaaattcattcacaaacctggcagttatatcttccggctgagctgtactcgtctgggtcagtgggctattgggtatgttactgctgatgggaacattctccagacaatccctcacaataaacctctcttccaagcactgattgatggcttcagggaaggcttctatttgtttcctgatggacgaaatcagaatcctgatctgactggcttatgtgaaccaactccccaagaccatatcaaagtgacccaggaacaatatgaattatactgtgagatgggctccacattccaactatgtaaaatatgtgctgaaaatgataaggatgtaaagattgagccctgtggacacctcatgtgcacatcctgtcttacatcctggcaggaatcagaaggtcagggctgtcctttctgccgatgtgaaattaaaggtactgaacccatcgtggtagatccgtttgatcctagagggagtggcagcctgttgaggcaaggagcagagggagctccctccccaaattatgatgatgatgatgatgaacgagctgatgatactctcttcatgatgaaggaattggctggtgccaaggtggaacggccgccttctccattctccatggccccacaagcttcccttcccccggtgccaccacgacttgaccttctgccgcagcgagtatgtgttccctcaagtgcttctgctcttggaactgcttctaaggctgcttctggctcccttcataaagacaaaccattgccagtacctcccacacttcgagatcttccaccaccaccgcctccagaccggccatattctgttggagcagaatcccgacctcaaagacgccccttgccttgtacaccaggcgactgtccctccagagacaaactgccccctgtcccctctagccgccttggagactcatggctgccccggccaatccccaaagtaccagtatctgccccaagttccagtgatccctggacaggaagagaattaaccaaccggcactcacttccattttcattgccctcacaaatggagcccagaccagatgtgcctaggctcggaagcacgttcagtctggatacctccatgagtatgaatagcagcccattagtaggtccagagtgtgaccaccccaaaatcaaaccttcctcatctgccaatgccatttattctctggctgccagacctcttcctgtgccaaaactgccacctggggagcaatgtgagggtgaagaggacacagagttcatgactccctcttccaggcctctacggcctttggatacatcccagagttcacgagcatgtgattgcgaccagcagattgatagctgtacgtttgaagcaatgtataatattcagtcccaggcgccatctatcaccgagagcagcacctttggtgaagggaatttggccgcagcccatgccaacactggtcccgaggagtcagaaaatgaggatgatgggtttgatgtcccaaagccacctgtgccggccgtgctggcccgccgaactctctcagatatctctaatgccagctcctcctttggctggttgtctctggatggtgatcctacaacaaatgtcactgaaggttcccaagttcccgagaggcctccaaaaccattcccgcggagaatcaactctgaacggaaagctggcagctgtcagcaaggtagtggtcctgccgcctctgctgccaccgcctcacctcagctctccagtgagatcgagaacctcatgagtcaggggtactcctaccaggacatccagaaagctttggtcattgcccagaacaacatcgagatggccaaaaacatcctccgggaatttgtttccatttcttctcctgcccatgtagctacctag

SEQ ID NO: 260 - protein, 906 aa, Artificial
  Synthetic Construct
MAGNVKKSSGAGGGTGSGGSGSGGLIGLMKDAFQPHHHHHHHLSPHPPGTVDKKMVEKCWKLMDKVVRLCQNPKLALKNSPPYILDLLPDTYQHLRTILSRYEGKMETLGENEYFRVFMENLMKKTKQTISLFKEGKERMYEENSQPRRNLTKLSLIFSHMLAELKGIFPSGLFQGDTFRITKADAAEFWRKAFGEKTIVPWKSFRQALHEVHPISSGLEAMALKSTIDLTCNDYISVFEFDIFTRLFQPWSSLLRNWNSLAVTHPGYMAFLTYDEVKARLQKFIHKPGSYIFRLSCTRLGQWAIGYVTADGNILQTIPHNKPLFQALIDGFREGFYLFPDGRNQNPDLTGLCEPTPQDHIKVTQEQYELYCEMGSTFQLCKICAENDKDVKIEPCGHLMCTSCLTSWQESEGQGCPFCRCEIKGTEPIVVDPFDPRGSGSLLRQGAEGAPSPNYDDDDDERADDTLFMMKELAGAKVERPPSPFSMAPQASLPPVPPRLDLLPQRVCVPSSASALGTASKAASGSLHKDKPLPVPPTLRDLPPPPPPDRPYSVGAESRPQRRPLPCTPGDCPSRDKLPPVPSSRLGDSWLPRPIPKVPVSAPSSSDPWTGRELTNRHSLPFSLPSQMEPRPDVPRLGSTFSLDTSMSMNSSPLVGPECDHPKIKPSSSANAIYSLAARPLPVPKLPPGEQCEGEEDTEFMTPSSRPLRPLDTSQSSRACDCDQQIDSCTFEAMYNIQSQAPSITESSTFGEGNLAAAHANTGPEESENEDDGFDVPKPPVPAVLARRTLSDISNASSSFGWLSLDGDPTTNVTEGSQVPERPPKPFPRRINSERKAGSCQQGSGPAASAATASPQLSSEIENLMSQGYSYQDIQKALVIAQNNIEMAKNILREFVSISSPAHVAT

SEQ ID NO: 261 - DNA, 1443 bp, Artificial
  c-Cbl 480
  CDS (1)..(1440)
atggccggcaacgtgaagaagagctctggggccgggggcggcacgggctccgggggctcgggttcgggtggcctgattgggctcatgaaggacgccttccagccgcaccaccaccaccaccaccacctcagcccccacccgccggggacggtggacaagaagatggtggagaagtgctggaagctcatggacaaggtggtgcggttgtgtcagaacccaaagctggcgctaaagaatagcccaccttatatcttagacctgctaccagatacctaccagcatctccgtactatcttgtcaagatatgaggggaagatggagacacttggagaaaatgagtattttagggtgtttatggagaatttgatgaagaaaactaagcaaaccataagcctcttcaaggagggaaaagaaagaatgtatgaggagaattctcagcctaggcgaaacctaaccaaactgtccctcatcttcagccacatgctggcagaactaaaaggaatctttccaagtggactctttcagggagacacatttcggattactaaagcagatgctgcggaattttggagaaaagcttttggggaaaagacaatagtcccttggaagagctttcgacaggctctacatgaagtgcatcccatcagttctgggctggaggccatggctctgaaatccactattgatctgacctgcaatgattatatttcggtttttgaatttgacatctttacccgactctttcagccctggtcctctttgctcaggaattggaacagccttgctgtaactcatcctggctacatggcttttttgacgtatgacgaagtgaaagctcggctccagaaattcattcacaaacctggcagttatatcttccggctgagctgtactcgtctgggtcagtgggctattgggtatgttactgctgatgggaacattctccagacaatccctcacaataaacctctcttccaagcactgattgatggcttcagggaaggcttctatttgtttcctgatggacgaaatcagaatcctgatctgactggcttatgtgaaccaactccccaagaccatatcaaagtgacccaggaacaatatgaattatactgtgagatgggctccacattccaactatgtaaaatatgtgctgaaaatgataaggatgtaaagattgagccctgtggacacctcatgtgcacatcctgtcttacatcctggcaggaatcagaaggtcagggctgtcctttctgccgatgtgaaattaaaggtactgaacccatcgtggtagatccgtttgatcctagagggagtggcagcctgttgaggcaaggagcagagggagctccctccccaaattatgatgatgatgatgatgaacgagctgatgatactctcttcatgatgaaggaattggctggtgccaaggtggaacggtag

SEQ ID NO: 262 - protein, 480 aa, Artificial
  Synthetic Construct
MAGNVKKSSGAGGGTGSGGSGSGGLIGLMKDAFQPHHHHHHHLSPHPPGTVDKKMVEKCWKLMDKVVRLCQNPKLALKNSPPYILDLLPDTYQHLRTILSRYEGKMETLGENEYFRVFMENLMKKTKQTISLFKEGKERMYEENSQPRRNLTKLSLIFSHMLAELKGIFPSGLFQGDTFRITKADAAEFWRKAFGEKTIVPWKSFRQALHEVHPISSGLEAMALKSTIDLTCNDYISVFEFDIFTRLFQPWSSLLRNWNSLAVTHPGYMAFLTYDEVKARLQKFIHKPGSYIFRLSCTRLGQWAIGYVTADGNILQTIPHNKPLFQALIDGFREGFYLFPDGRNQNPDLTGLCEPTPQDHIKVTQEQYELYCEMGSTFQLCKICAENDKDVKIEPCGHLMCTSCLTSWQESEGQGCPFCRCEIKGTEPIVVDPFDPRGSGSLLRQGAEGAPSPNYDDDDDERADDTLFMMKELAGAKVER

SEQ ID NO: 263 - DNA, 2721 bp, Homo sapiens
atggccggcaacgtgaagaagagctctggggccgggggcggcacgggctccgggggctcgggttcgggtggcctgattgggctcatgaaggacgccttccagccgcaccaccaccaccaccaccacctcagcccccacccgccggggacggtggacaagaagatggtggagaagtgctggaagctcatggacaaggtggtgcggttgtgtcagaacccaaagctggcgctaaagaatagcccaccttatatcttagacctgctaccagatacctaccagcatctccgtactatcttgtcaagatatgaggggaagatggagacacttggagaaaatgagtattttagggtgtttatggagaatttgatgaagaaaactaagcaaaccataagcctcttcaaggagggaaaagaaagaatgtatgaggagaattctcagcctaggcgaaacctaaccaaactgtccctcatcttcagccacatgctggcagaactaaaaggaatctttccaagtggactctttcagggagacacatttcggattactaaagcagatgctgcggaattttggagaaaagcttttggggaaaagacaatagtcccttggaagagctttcgacaggctctacatgaagtgcatcccatcagttctgggctggaggccatggctctgaaatccactattgatctgacctgcaatgattatatttcggtttttgaatttgacatctttacccgactctttcagccctggtcctctttgctcaggaattggaacagccttgctgtaactcatcctggctacatggcttttttgacgtatgacgaagtgaaagctcggctccagaaattcattcacaaacctggcagttatatcttccggctgagctgtactcgtctgggtcagtgggctattgggtatgttactgctgatgggaacattctccagacaatccctcacaataaacctctcttccaagcactgattgatggcttcagggaaggcttctatttgtttcctgatggacgaaatcagaatcctgatctgactggcttatgtgaaccaactccccaagaccatatcaaagtgacccaggaacaatatgaattatactgtgagatgggctccacattccaactatgtaaaatatgtgctgaaaatgataaggatgtaaagattgagccctgtggacacctcatgtgcacatcctgtcttacatcctggcaggaatcagaaggtcagggctgtcctttctgccgatgtgaaattaaaggtactgaacccatcgtggtagatccgtttgatcctagagggagtggcagcctgttgaggcaaggagcagagggagctccctccccaaattatgatgatgatgatgatgaacgagctgatgatactctcttcatgatgaaggaattggctggtgccaaggtggaacggccgccttctccattctccatggccccacaagcttcccttcccccggtgccaccacgacttgaccttctgccgcagcgagtatgtgttccctcaagtgcttctgctcttggaactgcttctaaggctgcttctggctcccttcataaagacaaaccattgccagtacctcccacacttcgagatcttccaccaccaccgcctccagaccggccatattctgttggagcagaatcccgacctcaaagacgccccttgccttgtacaccaggcgactgtccctccagagacaaactgccccctgtcccctctagccgccttggagactcatggctgccccggccaatccccaaagtaccagtatctgccccaagttccagtgatccctggacaggaagagaattaaccaaccggcactcacttccattttcattgccctcacaaatggagcccagaccagatgtgcctaggctcggaagcacgttcagtctggatacctccatgagtatgaatagcagcccattagtaggtccagagtgtgaccaccccaaaatcaaaccttcctcatctgccaatgccatttattctctggctgccagacctcttcctgtgccaaaactgccacctggggagcaatgtgagggtgaagaggacacagagtacatgactccctcttccaggcctctacggcctttggatacatcccagagttcacgagcatgtgattgcgaccagcagattgatagctgtacgtatgaagcaatgtataatattcagtcccaggcgccatctatcaccgagagcagcacctttggtgaagggaatttggccgcagcccatgccaacactggtcccgaggagtcagaaaatgaggatgatgggtatgatgtcccaaagccacctgtgccggccgtgctggcccgccgaactctctcagatatctctaatgccagctcctcctttggctggttgtctctggatggtgatcctacaacaaatgtcactgaaggttcccaagttcccgagaggcctccaaaaccattcccgcggagaatcaactctgaacggaaagctggcagctgtcagcaaggtagtggtcctgccgcctctgctgccaccgcctcacctcagctctccagtgagatcgagaacctcatgagtcaggggtactcctaccaggacatccagaaagctttggtcattgcccagaacaacatcgagatggccaaaaacatcctccgggaatttgtttccatttcttctcctgcccatgtagctacctag

SEQ ID NO: 264 - DNA, 42 bp, artificial
  synthetic oligonucleotide probe for detecting mouse NPY mRNA
gagggtcagtccacacagccccattcgcttgttacctagcat

SEQ ID NO: 265 - DNA, 45 bp, artificial
  synthetic oligonucleotide probe for detecting mouse cocaine- and amphetamine-regulated transcript mRNA
tccttctcgtgggacgcatcatccacggcagagtagatgtccagg

SEQ ID NO: 266 - DNA, 45 bp, artificial
  synthetic oligonucleotide probe for detecting mouse corticotropin-releasing hormone mRNA
ccgataatctccatcagtttcctgttgctgtgagcttgctgagct

SEQ ID NO: 267 - DNA, 45 bp, artificial
  synthetic oligonucleotide probe for detecting mouse thyrotropin-releasing hormone mRNA
aaccttactcctccagaggttccctgacccaggcttccagttgtg

SEQ ID NO: 268 - DNA, 19 bp, artificial
  synthetic oligonucleotide for knocking down mouse Cbl expression
gacactttccggattacta